(19) United States
(12) Patent Application Publication
(10) Pub. No.: US 2025/0213671 A1
(43) Pub. Date: Jul. 3, 2025

(54) NOVEL RNA AND DNA TECHNOLOGY FOR VACCINATION AGAINST ALPHAVIRUSES AND OTHER EMERGING AND EPIDEMIC VIRUSES

(71) Applicant: MEDIGEN, INC., Frederick, MD (US)

(72) Inventors: Peter PUSHKO, Frederick, MD (US); Irina TRETYAKOVA, Frederick, MD (US)

(21) Appl. No.: 18/848,494

(22) PCT Filed: Mar. 17, 2023
(86) PCT No.: PCT/US2023/015581

§ 371 Date: Sep. 18, 2024

Related U.S. Application Data
(60) Provisional application No. 63/321,652, filed on Mar. 18, 2022.

(51) Int. Cl. A61K 39/12; A61K 9/00; A61K 9/1272; A61K 9/51; A61K 39/00; A61P 31/14; B82Y 5/00; C12N 7/00

(52) U.S. Cl. CPC A61K 39/12; A61K 9/0021; A61K 9/1272; A61K 9/5123; A61P 31/14; C12N 7/00; A61K 2039/5254; A61K 2039/53; A61K 2039/54; A61K 2039/545; A61K 2039/55555; A61K 2039/70; B82Y 5/00; C12N 2770/36134; C12N 2770/36151

(57) ABSTRACT

An iRNA and/or iDNA vaccine system and/or platform are described for eliciting an immune response against one or more pathogens. Also described, are methods of making and using the iRNA and/or iDNA vaccine system and/or platform.

### CROSS-REFERENCE TO PRIOR APPLICATIONS

This application claims priority to U.S. Provisional Application No. 63/321,652, filed on Mar. 18, 2022, which is hereby expressly incorporated by reference in its entirety and assigned to the assignee hereof.

### GOVERNMENT INTERESTS

This subject matter was made in part with U.S. government support under Grant Number R43AI152717 awarded by the National Institute of Allergy and Infectious Diseases at the National Institutes of Health. The U.S. government may have certain rights in the subject matter.

### TECHNICAL FIELD

An iRNA and/or iDNA vaccine system and/or platform for eliciting an immune response against one or more pathogens and methods of making and using the novel system and/or platform.

### BACKGROUND

Various vaccine systems or platforms have been proposed. Because these vaccine systems or platforms are not optimal, there is a need in the field for improved systems or platforms, including effective, safe and economical systems or platforms.

### BRIEF DESCRIPTION OF THE DRAWINGS

FIG. 1 shows countries with current or previous local transmission of CHIKV as reported by the CDC. Countries where only imported cases have been documented are not included.

FIG. 2 shows an overview of iRNA immunization. The rearranged CHIKV iRNA is placed downstream from optimized hybrid hCMV promoter sequences. In tissues injected with iDNA, transcription from the promoter yields the full-length rearranged iRNA capable of initiating limited replication of the live attenuated virus particles that induces specific immune responses.

FIG. 3 shows genetic structures of the prototype 181/25 virus (top), rearranged CHIKV iRNA (middle), and iDNA plasmid encoding rearranged iRNA. Attenuating mutations are shown with asterisks, two 26S promoters are shown with open arrows.

FIGS. 4A and 4B show genetic maps depicting the pMG5040 plasmid encoding the full-length rearranged RNA. FIG. 4A shows the approximate locations of the CMV promoter (solid arrow), 26S promoter (open arrows) and attenuating mutations (asterisks). The wild type CHIKV genome is shown on the top panel. CHIKV pMG5040 plasmid encoded rearranged full-length infectious CHIKV RNA under transcriptional control of the CMV promoter is shown on the middle panel. The plasmid pMG5040 contained both attenuating mutations from the IND vaccine 181/25 is shown on the bottom panel. FIG.4B shows 1% Agarose/TAE gel of pMG5040 plasmid as compared to control VEEV vaccine. Lane M is the 1 KB Plus DNA ladder purchased from Thermo; lane 1 is the VEE control pMG4020; and lane 2 is the CHIKV pMG5040.

FIG.5 shows induction of immunity using iRNA. The vaccines induce innate immunity and prime adaptive immune responses to live-attenuated, rearranged CHIKV iRNA.

FIG. 6 shows growth curves of CHIKV vaccine in Vero cells infected with virus (dashed lines) or transfected with iDNA (solid lines) Vero cells. Cells were infected with 181/25 virus or transfected by electroporation with preparations of 181/25-encoding plasmids #7 and #39.

FIGS. 7A and 7B show the preparation of V5040 CHIKV in Vero cells. FIG. 7A shows the plaque morphology of attenuated V5040 and 181/25 vaccine viruses. FIG. 7B shows the comparison of the growth kinetics of V5040 in Vero cell infected with V5040 or transfected with pMG5040, with CHIKV 181/25 vaccine virus being used as a control.

FIGS. 8A and 8B show the immunogenicity of V5040 virus in mice FIG. 8A shows a western blot with antisera from BALB/c mice vaccinated with V5040 virus. Mouse serum was probed at 1:100 dilution followed by AP-conjugated goat anti-mouse lgG (H+L) secondary antibody (1:1000 dilution). Lane 1 shows the SeeBlue Plus2 standard; Lane 2 shows the control Vero cell lysate; Lane 3 shows the blank; and Lane 4 shows the lysate of Vero cells infected with CHIKV 181/25 virus. Predicted band of E2 is indicated. FIG. 8B shows IFA of Vero cells with antisera from BALB/c mice vaccinated with V5040 virus. Vero cells were seeded in chamber slides were infected with V5040 at MOI 0.01. At 24 h post-infection, Infected monolayer cells were fixed with acetone and probed with mouse serum diluted 1:25, followed by FITC-conjugated goat anti-mouse IgG (H+L) secondary antibody (1:25 dilution). Infected Vero monolayers developed foci of V5040-infected cells stained in green (indicated with arrow). Nuclei are stained in red using VectaShield mounting medium containing propidium iodide.

FIG. 9 shows the neutralization antibodies from C57BL/6 mice vaccinated with V5040 or CHIKV 181/25. Antisera collected from terminal bleeding of mice vaccinated either with V5040 or CHIKV 181/25 were tested for PRNT50 assays against their corresponding homologous virus. Circles and squares indicate antisera of CHIKV 181/25 and V5040, respectively. Each circle or square represents serum from an individual mouse (n=8/group/timepoint). The bar and whiskers indicate the means and ±their standard deviation. Ns means not significant. **means P<0.0021 as determined by Two-Way ANOVA with Sidak's multiple comparisons test (Prism).

FIG. 10 shows a Table 1 describing CHIKV 181/25 iDNA Vaccine in BALB/c Mice.

FIG. 11 shows a Table 2 describing the immunogenicity of V5040 CHIKV vaccine in BALB/c and C57BL/6 mice. PFU means plaque-forming units; s.c. means subcutaneously; WB means western blot; IFA means indirect immunofluorescense assay; PRNT50 means plaque reduction neutralization assay; and nt means not tested.

FIG. 12 shows the genetic structure of the pMG4020 plasmid encoding the V4020 VEEV vaccine virus with rearranged genome, including the location of the 5′ cap, the major open reading frames (nsP1-4, GP, C), the 26S subgenomic promoters (open arrows), the 3′-Poly (A), and the attenuation mutations in asterisks. The diagram is not to scale.

FIG. 13 shows the immunogenicity of pMG4020 and V4020 vaccines in rabbits. Pre-bleed sera (day 0) were used to compare PRNT titers of sera from days 7 and 21 post-vaccination to PRNT titers of pre-bleed. Statistical analysis was performed using mean logarithm of titer (day 21) and one-sided Student's t-test. V4020 means live-attenuated virus; pMG4020 means iDNA plasmid; SC means subcutaneously using syringe; TD means transdermally using 3M (Kindeva) hMTS devices; PRNT means plaque reduction neutralization test.

### SUMMARY

Described herein are several iRNA and/or iDNA vaccine systems and/or platforms for eliciting an immune response against one or more pathogens and methods of making and using the novel system or platform.

Disclosed herein is an infections RNA (iRNA) capable of directly launching a live-attenuated alphavirus in vivo and directly eliciting an immune response in vivo following administration of the iRNA. Exemplary embodiments of alphavirus include CHIKV, VEEV, YFV, JEV, SARS-COV-2, and combinations thereof. Exemplary embodiments also include emerging variant thereof.

The iRNA is characterized as being attenuated and not virulent when administering to a subject in need thereof. The attenuation is achieved by one or more mutations, including at least one point mutations (such as attenuating point mutations) and at least one rearrangements. In preferred embodiments, the rearranged is achieved in a laboratory, preferably by a skilled genetic engineer. Preferably the rearrangement is between the capsid protein and the glycoprotein. Because designed-in rearrangement in the iRNA would not be reversed during pharmaceutical production and/or following administration into a subject in need thereof, the iRNA is characterized as been safely and stably attenuated, with negligible risk of reversion to the more virulent viral strains. In exemplary embodiments, the iRNA encodes the full length live attenuated alphavirus. In exemplary embodiments, the iRNA achieves safely and stably attenuated without the need to delete of large portions of the genome of the alphavirus. A deletion of a large portion of the genome is, for example, a deletion of about 60 bp.

In exemplary embodiments, the iRNA is further characterized as having a ultra-low effective vaccine dose for eliciting a robust immune response in a subject in need thereof. Because the iRNA is infectious and capable of self-replication in the host cell, it can elicit a robust immune response with the administration of nanograms of the iRNA. In exemplary embodiments, the iRNA, alone is capable of eliciting a robust immune response. That is, the iRNA can elicited a robust immune response without the need of an iDNA, a vehicle (such as a liposome) carrying the iRNA, a VLP, and/or a viral vector. In exemplary embodiments, the iRNA can be combined with a non-toxic pharmacological excipient and/or an non-toxic adjuvant suitable for administering in a subject in need thereof. In exemplary embodiments, the iRNA can be encapsulated in a nanoparticle, such as a lipid nanoparticle, including a liposome. In other exemplary embodiments, the iRNA can be administered to the subject in directly, through a corresponding iDNA encoding the iRNA. In certain embodiments, the iRNA is administered by injection in a suitable location on the subject. In other embodiments, the iRNA is administered by microneedle or using a needleless approach. In certain embodiments different iRNAs and iDNAs can be combined to create a multivalent composition capable of eliciting an immune response in a subject against one or more, if not all, of the different encoded viruses. In another embodiment, the different iRNAs and iDNAs can be encased in a nanoparticle, such as a lipid nanoparticle, to achieve the same effect.

In an exemplary embodiment, the iRNA is produced using GMP practices and in certain embodiments, the iRNA is in a pharmaceutically acceptable formulation for administration to a subject in need thereof.

Disclosed herein is a novel iRNA and methods of making and using thereof. The infectious RNA (iRNA) molecule can include an RNA encoding an attenuated virus. The attenuated virus can be an emerging virus. The attenuated virus can be a positive-strand RNA virus, preferably a positive-strand RNA virus selected from the group consisting of one or more Ribovirus, Orthornavirus, Kitrinovirus, Lenarvirus, Pisuvirus, Pisonivirus, Stelpavirus, Togavirus, and combinations thereof; more preferably selected from the group consisting of:
one or more Alphavirus such as western equine encephalitis virus (WEEV), Venezuelan equine encephalitis virus (VEEV), eastern equine encephalitis virus (EEEV), Sindbis virus (SINV), Semliki Forest virus (SFV), and Chikungunya virus (CHIKV),
one or more Flavivirus, such as Hepacivirus C, yellow fever virus (YFV), West Nile virus, Dengue virus, and Japanese encephalitis virus (JEV),
one or more Arenavirus, such as Lassa virus, Guanarito virus, Junin virus, Lujo virus. Machupo virus, Sabia virus, and Whitewater Arroyo virus,
one or more Coronavirus, such as Middle East respiratory syndrome (MERS) virus, Severe Acute Respiratory Syndrome (SARS) virus, and SARS-Coronavirus 2 (SARS-COV-2),
one or more Picornavirus, such as rhinovirus and common cold virus,
one or more retrovirus, such as simian immunodeficiency virus (SIV), human immunodeficient virus (HIV), simian type D retrovirus (SRV), and simian T-lymphotropic virus (STLV),
and combinations thereof.
   In certain embodiments, the positive-strand RNA virus is selected from the group consisting of one or more CHIKV, VEEV, YFV, JEV, and combinations thereof. In certain other embodiments, the positive-strand RNA virus is selected from the group consisting of one or more CHIKV, VEEV, and combinations thereof. In certain preferred embodiments, the positive-strand RNA virus is a CHIKV. In certain other preferred embodiments, the positive-strand RNA virus is VEEV. In certain preferred embodiments, the positive-strand RNA virus is selected from the group consisting of a 181/25 CHIKV, a V5040 CHIKV, a TC85 VEEV, V4020 VEEV, and combinations thereof. The attenuated virus can be encoded by the sequence of any one of SEQ. 1 to 4.

The iRNA molecule disclosed herein can encode all of the gene products of the attenuated virus, preferably encoding a full-length RNA from the attenuated virus. The iRNA molecule is preferably a recombinant RNA molecule, including an iRNA molecule that encodes one or more attenuating mutations. The one or more attenuating mutations can be a point mutation, a rearrangement, and/or combinations thereof. In particular, the one or more attenuating mutation excludes any non-rearrangement deletion of more than 180 bp, preferably no more than about 175 bp, more preferably no more than about 170 bp, more preferably no more than about 170 bp, more preferably no more than about 165 bp, more preferably no more than about 160 bp, more preferably no more than about 155 bp, more preferably no more than about 150 bp, more preferably no more than about 125 bp, more preferably no more than about 100 bp, more preferably no more than about 75 bp, more preferably no more than about 55 bp, more preferably no more than about 50 bp, more preferably no more than about 25 bp, more preferably no more than about 20 bp, more preferably no more than about 15 bp, more preferably no more than about 10 bp, more preferably no more than about 5 bp, more preferably no more than about 0 bp. In certain preferred embodiment, the one or more attenuating mutation excludes a non-rearrangement deletion of nucleotides encoding no more than 58 amino acids, preferably no more than about 55 amino acids, more preferably no more than about 50 amino acids, more preferably no more than about 45 amino acids, more preferably no more than about 40 amino acids, more preferably no more than about 35 amino acids, more preferably no more than about 30 amino acids, more preferably no more than about 25 amino acids, more preferably no more than about 20 amino acids, more preferably no more than about 15 amino acids, more preferably no more than about 10 amino acids, more preferably no more than about 5 amino acids, more preferably no more than about 0 amino acids.

In certain preferred embodiments the iRNA molecule encodes a full-length nsP3 gene when the attenuated virus is a CHIKV and/or a full-length 6K gene when the attenuated virus is a CHIKV. In certain preferred embodiments, iRNA molecule encodes two attenuating point mutations and/or a rearrangement. The rearrangement can be between the capsid gene and the glycoprotein gene. The one or more of the point mutation can be at the glycoprotein gene and/or at the envelop gene. The one or more point mutation can be at the E2 gene of the CHIKV. Exemplary attenuating point mutations include one or both of a Thr12Ile and Gly82Arg mutation of the E2 gene of the CHIKV. In other exemplary embodiments, the one or more mutations is selected from in SEQ. 1 and/or SEQ 3.

The designed-in mutation in the iRNA is substantially stable. It has

substantially reduced rates of reversion, is substantially resistant to reversions, and/or combinations thereof. Preferably the mutation is highly stable, has highly reduced rates of reversion, is highly resistant to reversions, and/or combinations thereof. Preferably the mutation does not reverse to a more virulent virus. Preferably the mutation has no detectable reversions.

In certain preferred embodiments, the designed-in mutation is substantially stable, or highly stable, or has no detectable reversions following a direct or an indirect administration of the iRNA into a subject in need thereof. Preferably the mutation is substantially stable, or highly stable, or has no detectable reversions during the entire course of treatment following a direct or an indirect administration of the iRNA into a subject in need thereof.

In certain preferred embodiments, the mutation does not impair the replication of attenuated virus in a mammalian cell, preferably a primate cell, more preferably a human cell. In certain preferred embodiments, the mutation is stable.

In certain preferred embodiments, the iRNA molecule comprises one or more subgenomic promoters. The one or more, preferably two or more, subgenomic promoters are operably linked to the iRNA molecule. Preferably the one or more subgenomic promoters is an RNA polymerase promoter, preferably an eukaryotic promoter or a bacteriophage promoter. Preferably the one or more subgenomic promoters is one or more 26S promoters. Preferably the one or more subgenomic promoters is two 26S promoters. Preferably each subgenomic promoter is operably linked to a gene encoded by the iRNA molecule. Preferably the first subgenomic promoter is operatively linked to a capsid gene and the second subgenomic promoter is operatively linked to a glycoprotein gene. Preferably the glycoprotein gene is E3-E2-6K-E1. In certain preferred embodiments, the iRNA molecule has a sequence of SEQ. 1 and/or SEQ. 3.

Disclosed herein is an infectious DNA (iDNA) molecule encoding any one of the iRNA molecule disclosed herein. The iDNA is operatively linked to a DNA-dependent RNA polymerases production promoter. In certain preferred embodiments, the production promoter is suitable for manufacturing the encoded iRNA molecule. Preferably for the iRNA molecule manufactured is pharmaceutically acceptable. In certain preferred embodiments, the production promoter is a bacteriophage promoter or a prokaryotic promoter. In certain embodiments, the promoter is selected from the group consisting of T3, T7 and 26S. In certain preferred embodiments, the production promoter is distinct from the subgenomic promoter. In certain preferred embodiments, the iDNA molecule is carried in a DNA plasmid. In certain preferred embodiments, the iDNA molecule is carried in a pT7 plasmid. In certain preferred embodiments, the iDNA molecule is carried in a pMG plasmid. In preferred embodiments, the DNA plasmid is a pMG4020 plasmid and/or a pMG5040 plasmid. In certain preferred embodiments, the iDNA has the a sequence of SEQ. 2 and/or SEQ. 4.

The iDNA can further comprise an eukaryotic promoter, preferably the promoter is optimized and preferably the promoter is a mammalian promoter, more preferably a CMV promoter, and more preferably an optimized hybrid human CMV (hCMV) promoter.

In certain preferred embodiments, the iDNA molecule encodes a full-length RNA from the attenuated virus. In certain preferred embodiments, the eukaryotic promoter and/or production promoter is located upstream of the iRNA encoded by the iDNA molecule. In certain preferred embodiments, the iDNA molecule is stable, preferably thermal and/or genetically stable.

In certain preferred embodiments, the iDNA molecule is characterized by being sterile, and/or having about 95% of the iDNA molecule being supercoiled, and/or having an A₂₆₀/A₂₈₀ ratio of selected from the group consisting of from about 1.6 to about 2.2, from about 1.7 to about 2.1, from about 1.8 to about 2.0, and about 1.9. In certain preferred embodiments, the iDNA molecule is capable of inducing an immune response, preferably the immune response is an innate immune response, an adaptive immune response, and/or combinations thereof; more preferably the immune response is a long-lasting immunity. The acquired immunity is preferably a broadly cross-neutralizing immune response. In a preferred embodiment, the iDNA molecule is capable of inducing the immune response in a single dose.

In certain preferred embodiments, the iDNA molecule is pharmaceutically acceptable. The iDNA molecule can be manufactured in vitro or in vivo or by direct synthesis. Preferably, the iDNA molecule is manufactured in vitro, under GMP conditions, and manufactured for vaccine use.

In certain preferred embodiments, the iDNA is formulated in a composition further comprising a pharmaceutically acceptable non-toxic component. The component can be a saline and/or a buffer; and preferably the saline is a phosphate buffered solution. In certain embodiments, the component is a carrier, preferably a pharmaceutically acceptable carrier. The preferred composition containing the iDNA is preferably is pharmaceutically acceptable.

In certain preferred embodiments, the composition and/or iDNA molecule is suitable for direct administration in a subject in need thereof. In certain preferred embodiments, the composition and/or the iDNA molecule is capable of inducing an immune response in a subject in need thereof through an iRNA, preferably the immune response is an innate immune response, or an adaptive immune response, and/or combinations thereof; more preferably the immune response is a long-lasting immunity; more preferably the immune response is a broadly cross-neutralizing immune response. In a preferred embodiment, the composition and/or the iDNA molecule is capable of inducing the immune response in one or more doses, preferably a single dose.

In certain preferred embodiments, the composition and/or the iDNA molecule is suitable for a use as a vaccine. In certain preferred embodiments, the iDNA molecule is suitable for a use as a vaccine. In certain preferred embodiments, the virus is suitable for preventing and/or reducing and/or substantially minimizing one or more symptoms in a subject in need thereof. The subject is preferably a mammal, more preferably a human.

In certain preferred embodiments, the iDNA molecule is used to prevent, reduce or substantially minimize a viral infection and/or prevent or diminish the symptoms of a viral infection; and preferably the symptoms are selected from the group consisting of a fever, headache, rash, nausea, myalgia, arthralgia, respiratory failure, cardiovascular disease, hepatitis, cutaneous effects, central nervous system abnormalities, and combinations thereof.

Disclosed herein is a composition comprising the iRNA molecule disclosed herein, further comprising a pharmaceutically acceptable non-toxic component. The component can be a saline and/or a buffer; and preferably the saline is a phosphate buffered solution. In certain preferred embodiments, the iRNA molecule is pharmaceutically acceptable. In certain preferred embodiments, the iRNA molecule is manufactured in vitro or in vivo or by direct synthesis; preferably, iRNA molecule is manufactured in vitro, under GMP conditions, and is manufactured for vaccine use.

In certain preferred embodiments, the iRNA molecule is manufactured using the iDNA disclosed herein, preferably the composition is pharmaceutically acceptable. In certain preferred embodiments, the composition and/or iRNA molecule is suitable for direct administration in a subject in need thereof. In certain preferred embodiments, the composition and/or the iRNA molecule is capable of inducing an immune response following direct administration in a subject in need thereof, preferably the immune response is an innate immune response, or an adaptive immune response, and/or combinations thereof; preferably the immune response is a long-lasting immunity; preferably the immune response is a broadly cross-neutralizing immune response. In a preferred embodiment, the composition and/or the iRNA molecule is capable of inducing the immune response in one or more doses, preferably a single dose.

In certain preferred embodiments, the composition and/or the iRNA molecule is suitable for a use as a vaccine. In certain preferred embodiments, the composition and/or the iRNA molecule is suitable for preventing and/or reducing and/or substantially minimizing one or more symptoms in a subject in need thereof. The subject is preferably a mammal, more preferably a human. In certain preferred embodiments, the iRNA molecule is used to prevent, reduce or substantially minimize a viral infection and/or prevent or diminish the symptoms of a viral infection. Preferably the symptoms is selected from the group consisting of a fever, headache, rash, nausea, myalgia, arthralgia, respiratory failure, cardiovascular disease, hepatitis, cutaneous effects, central nervous system abnormalities, and combinations thereof.

Disclosed herein is a vehicle comprising an infectious genetic material. In certain preferred embodiments, the vehicle encapsulates the infectious genetic material. In certain preferred embodiments, the infectious genetic material comprise the iRNA molecule disclosed herein, the iDNA molecule disclosed herein, the composition disclosed herein, and/or combinations thereof. In certain preferred embodiments, the vehicle is a nanoparticle, including a liposome and a lipid nanoparticle, and including a cationic lipid 1,2-dioleoyl-3-timethylammonium-propane (DOTAP) lipid-based lipid nanoparticle. In certain preferred embodiments, the lipid nanoparticle comprising the infectious genetic material is formed by mixing the iRNA molecule with a PEGylated and cationic lipid.

In certain preferred embodiments, the lipid nanoparticle encapsulate the initial infectious genetic material at an amount of more than about 60%, preferably more than about 70% preferably more than about 80%, preferably more than about 90%, preferably more than about 91%, preferably more than about 92%, preferably more than about 93%, preferably more than about 94%, preferably more than about 95%, preferably more than about 96%, preferably more than about 97%, preferably more than about 98%, preferably more than about 99%, preferably more than about 99.9%. In certain preferred embodiments, the lipid nanoparticle has an average diameter of about 5 nm to about 200 nm; preferably about 10 nm to about 150 nm; preferably about 20 nm to about 100 nm; preferably about 30 nm to about 90 nm; preferably about 40 nm to about 85 nm; preferably about 50 nm to about 80 nm; preferably about 60 nm to about 75 nm; preferably about 75 nm. In certain preferred embodiments, the lipid nanoparticle are unilamellar vesicles. In certain preferred embodiments, the lipid nanoparticle comprise infectious genetic material per lipid nanoparticle at a range selected from the group consisting of about 1 to about 100, about 1 to about 90, about 1 to about 80, about 1 to about 70, about 1 to about 60, about 1 to about 50, about 1 to about 40, about 1 to about 30, about 1 to about 20, about 1 to about 10, about 1 to about 5, about 1 to about 4, about 1 to about 3, about 1 to about 2, about 1 infectious genetic material per lipid nanoparticle.

In certain preferred embodiments, the vehicle is capable of delivering the infectious genetic material to a subject in need thereof. In certain preferred embodiments, the vehicle is capable of delivering the infectious genetic material to the cytoplasm of a cell of a subject in need thereof. In certain preferred embodiments, the vehicle is capable of inducing an immune response, preferably an innate immune response, an adaptive immune response, and/or combinations thereof; preferably a long-lasting immunity; and

preferably a broadly cross-neutralizing immune response.

In certain preferred embodiments, the vehicle is capable of inducing the immune response in a single dose. In certain preferred embodiments, the vehicle is suitable for vaccine use. In certain preferred embodiments, the vehicle further comprises a non-toxic excipient thereof and/or an non-toxic adjuvant.

In certain preferred embodiments, the vehicle comprising the infectious genetic material is stable for storage at ambient temperatures and/or ultra-cold temperatures;

more preferably the vehicle comprising the infectious genetic material is stable for storage without the need for ultra-cold temperatures. In certain preferred embodiments, the vehicle comprising the infectious genetic material is stable for more than 12 weeks following lyophilization, such as from about 12 weeks to about 52 weeks, and from about 12 weeks to about 24 weeks.

Disclosed herein is a virus comprising an infectious genetic material. The infectious genetic material is the iRNA molecule disclosed herein, the iDNA molecule disclosed herein, the composition of disclosed herein, and/or combinations thereof. In certain embodiment, the virus is a recombinant virus, is homogenously pure, is live-attenuated, and/or contains stable and attenuating mutations.

In certain preferred embodiments, the virus is capable of inducing an immune response, preferably the immune response is an innate immune response, an adaptive immune response, and/or combinations thereof; more preferably the immune response is a long-lasting immunity; and more preferably the immune response is a broadly cross-neutralizing immune response. In certain preferred embodiments, the virus is capable of inducing the immune response in a single dose.

In certain preferred embodiments, the virus is suitable for use in a vaccine. In certain preferred embodiments, the virus is suitable for preventing and/or reducing and/or substantially minimizing one or more symptoms in a subject in need thereof. The subject is preferably a mammal, more preferably a human; preferably the virus is suitable for administration by injection, including intramuscular injection, and/or, by subcutaneous injection, and/or combinations thereof. In certain preferred embodiments, the vehicle is used to prevent, reduce or substantially minimize a viral infection and/or prevent or diminish the symptoms of a viral infection. In certain preferred embodiments, the symptoms is selected from the group consisting of a fever, headache, rash, nausea, myalgia, arthralgia, respiratory failure, cardiovascular disease, hepatitis, cutaneous effects, central nervous system abnormalities, and combinations thereof.

Disclosed herein is an vaccine comprising (i) an infectious genetic material and/or a vehicle and (ii) a non-toxic pharmacological excipient thereof. In certain embodiments the pharmacological excipient can be a pharmacologically acceptable carrier. The infectious genetic material is the iRNA molecule disclosed herein, the iDNA molecule disclosed herein, the composition disclosed herein, the vehicle disclosed herein, and/or combinations thereof. In certain preferred embodiments, the infectious genetic material is homogenously pure. In certain preferred embodiments, the infectious genetic material has a low percent of single nucleotide polymorphism. In certain preferred embodiments, the pharmacological excipient comprise a preservative and/or a saline; and preferably the saline is a phosphate buffered solution. In certain preferred embodiments, the vaccine further comprise an adjuvant. In certain preferred embodiments, the vaccine is a multivalent vaccine.

In certain preferred embodiments, the vaccine comprise an effective amount the attenuated virus. In certain preferred embodiments, the effective dose of the vaccine is selected from the group consisting of from about 1 ng to about 500,000 ng, from about 5 ng to about 250,000 ng, from about 6 ng to about 100,000 ng, from about 7 ng to about 50,000 ng, from about 8 ng to about 10,000 ng, from about 9 ng to about 5,000 ng, from about 10 ng to about 1,000 ng, from about 10 ng to about 500 ng, from about 10 ng to about 250 ng, from about 10 ng to about 100 ng, from about 100 ng, from about 10 ng to about 50 ng, from about 50 ng to about 500 ng, from about 50 ng to about 1,000 ng, from about 100 ng to about 1,000 ng, from about 100 ng to about 5,000 ng, from about 100 ng to about 10,000 ng. from about 500 ng to about 1,000 ng, from about 500 ng to about 5,000 ng, from about 500 ng to about 10,000 ng, from about 500 ng to about 50,000 ng, from about 500 ng to about 75,000 ng, and from about 500 ng to about 100,000 ng of the infectious genetic material.

In certain preferred embodiments, the effective dose of the vaccine is selected from the group consisting of from about 1 ng to about 500,000 ng, from about 5 ng to about 250,000 ng, from about 6 ng to about 100,000 ng, from about 7 ng to about 50,000 ng, from about 8 ng to about 10,000 ng, from about 9 ng to about 5,000 ng, from about 10 ng to about 100,000 ng, from about 10 ng to about 75,000 ng, from about 10 ng to about 50,000 ng, from about 10 ng to about 25,000 ng, from about 10 ng to about 10,000 ng. from about 10 ng to about 7,500 ng, from about 10 ng to about 50,000 ng, from about 10 ng to about 25,000 ng, from about 10 ng to about 10,000 ng, from about 10 ng to about 7,500 ng, from about 10 ng to about 5,000 ng, from about 10 ng to about 2,500 ng, from about 10 ng to about 1,000 ng, from about 10 ng to about 500 ng, from about 10 ng to about 250 ng, from about 10 ng to about 120 ng, from about 10 ng to about 100 ng, from about 10 ng to about 50 ng, from about 10 ng to about 75 ng, from about 50 ng to about 500 ng, from about 50 ng to about 1,000 ng, from about 100 ng to about 1,000 ng, from about 100 ng to about 5,000 ng, from about 100 ng to about 10,000 ng. from about 500 ng to about 1,000 ng, from about 500 ng to about 5,000 ng, from about 500 ng to about 10,000 ng, from about 500 ng to about 50,000 ng, from about 500 ng to about 75,000 ng, and from about 500 ng to about 100,000 ng of the iRNA molecule.

In certain preferred embodiments, the effective dose of the vaccine is

selected from the group consisting of from about 1 ng to about 500,000 ng, from about 5 ng to about 250,000 ng, from about 6 ng to about 100,000 ng, from about 7 ng to about 50,000 ng, from about 8 ng to about 10,000 ng, from about 9 ng to about 5,000 ng, from about 10 ng to about 1,200 ng, from about 10 ng to about 1,100 ng, from about 10 ng to about 1,000 ng, from about 10 ng to about 1,200 ng, from about 10 ng to about 500 ng, from about 10 ng to about 250 ng, from about 10 ng to about 120 ng, from about 10 ng to about 100 ng, from about 10 ng to about 50 ng, from about 50 ng to about 500 ng, from about 50 ng to about 1,000 ng, from about 100 ng to about 1,000 ng, from about 100 ng to about 1,100 ng, from about 100 ng to about 1,200, from about 100 ng to about 5,000 ng, from about 100 ng to about 10,000 ng, from about 500 ng to about 1,000 ng, from about 500 ng to about 5,000 ng, from about 500 ng to about 10,000 ng, from about 500 ng to about 50,000 ng, from about 500 ng to about 75,000 ng, and from about 500 ng to about 100,000 ng of the iDNA molecule.

In certain preferred embodiments, the effective dose of the vaccine is selected from the group consisting of from about 0.5 ng to about 100,000 ng, from about 0.5 ng to about 75,000 ng, from about 0.5 ng to about 50,000 ng, from about 0.5 ng to about 25,000 ng, from about 0.5 ng to about 10,000 ng, from about 0.5 ng to about 7,500 ng, from about 0.5 ng to about 5,000 ng, from about 0.5 ng to about 2,500 ng, from about 0.5 ng to about 1,000 ng, from about 0.5 ng to about 500 ng, from about 0.5 ng to about 250 ng, from about 0.5 ng to about 120 ng, from about 1 ng to about 100,000 ng, from about 1 ng to about 75,000 ng, from about 1 ng to about 50,000 ng, from about 1 ng to about 25,000 ng, from about 1 ng to about 10.000 ng, from about 1 ng to about 7,500 ng, from about 1 ng to about 5,000 ng, from about 1 ng to about 2,500 ng, from about 1 ng to about 1,000 ng, from about 1 ng to about 500 ng, from about 1 ng to about 250 ng, from about 1 ng to about 120 ng, from about 10 ng to about 120 ng, from about 10 ng to about 119 ng, from about 10 ng to about 118 ng, from about 10 ng to about 117 ng, from about 10 ng to about 116 ng, from about 10 ng to about 115 ng, from about 10 ng to about 114 ng, from about 10 ng to about 113 ng, from about 10 ng to about 112 ng, from about 10 ng to about 111 ng, from about 10 ng to about 110 ng, from about 10 ng to about 109 ng, from about 10 ng to about 108 ng, from about 10 ng to about 107 ng, from about 10 ng to about 106 ng, from about 10 ng to about 105 ng, from about 10 ng to about 104 ng, from about 10 ng to about 103 ng, from about 10 ng to about 102 ng, from about 10 ng to about 101 ng, from about 10 ng to about 100 ng, from about 15 ng to about 120 ng, from about 15 ng to about 119 ng, from about 20 ng to about 118 ng, from about 25 ng to about 117 ng, from about 30 ng to about 116 ng, from about 40 ng to about 115 ng, from about 50 ng to about 114 ng, from about 60 ng to about 113 ng, from about 70 ng to about 112 ng, from about 80 ng to about 111 ng, from about 90 ng to about 110 ng of the iDNA molecule, wherein the iDNA molecule encodes a live-attenuated CHIKV.

In certain preferred embodiments, the effective dose of the vaccine is selected from the group consisting of from about 0.5 ng to about 100,000 ng, from about 0.5 ng to about 75,000 ng, from about 0.5 ng to about 50,000 ng, from about 0.5 ng to about 25,000 ng, from about 0.5 ng to about 10,000 ng, from about 0.5 ng to about 7,500 ng, from about 0.5 ng to about 5,000 ng, from about 0.5 ng to about 2,500 ng, from about 0.5 ng to about 1,000 ng, from about 0.5 ng to about 500 ng, from about 0.5 ng to about 250 ng, from about 0.5 ng to about 120 ng, from about 1 ng to about 100,000 ng, from about 1 ng to about 75,000 ng, from about 1 ng to about 50,000 ng, from about 1 ng to about 25,000 ng, from about 1 ng to about 10,000 ng, from about 1 ng to about 7,500 ng, from about 1 ng to about 5,000 ng, from about 1 ng to about 2,500 ng, from about 1 ng to about 1,000 ng, from about 1 ng to about 500 ng, from about 1 ng to about 250 ng, from about 1 ng to about 120 ng, from about 10 ng to about 120 ng, from about 10 ng to about 119 ng. from about 10 ng to about 118 ng, from about 10 ng to about 117 ng, from about 10 ng to about 116 ng, from about 10 ng to about 115 ng, from about 10 ng to about 114 ng, from about 10 ng to about 113 ng, from about 10 ng to about 112 ng, from about 10 ng to about 111 ng, from about 10 ng to about 110 ng, from about 10 ng to about 109 ng, from about 10 ng to about 108 ng, from about 10 ng to about 107 ng, from about 10 ng to about 106 ng, from about 10 ng to about 105 ng, from about 10 ng to about 104 ng, from about 10 ng to about 103 ng, from about 10 ng to about 102 ng, from about 10 ng to about 101 ng, from about 10 ng to about 100 ng, from about 15 ng to about 120 ng, from about 15 ng to about 119 ng, from about 20 ng to about 118 ng, from about 25 ng to about 117 ng, from about 30 ng to about 116 ng, from about 40 ng to about 115 ng, from about 50 ng to about 114 ng, from about 60 ng to about 113 ng, from about 70 ng to about 112 ng, from about 80 ng to about 111 ng, from about 90 ng to about 110 ng of the iRNA molecule, wherein the iRNA molecule encodes a live-attenuated CHIKV.

In certain preferred embodiments, the effective dose of the vaccine is

selected from the group consisting of from about 1 ng to about 200,000 ng, from about 2 ng to about 150,000 ng, from about 3 ng to about 100,000 ng, from about 4 ng to about 90,00 ng, from about 5 ng to about 80,000 ng, from about 6 ng to about 70,000 ng, from about 7 ng to about 60,000 ng, from about 8 ng to about 50,000 ng, from about 9 ng to about 40,000 ng, from about 10 ng to about 30,000ng, from about 10 ng to about 20,000ng, from about 10 ng to about 19,000ng, from about 10 ng to about 18,000 ng, from about 10 ng to about 17,000 ng, from about 10 ng to about 16,000ng. from about 10 ng to about 15,000 ng, from about 10 ng to about 10,000ng, from about 10 ng to about 5,000 ng, from about 10 ng to about 1,000 ng, from about 10 ng to about 500 ng, from about 100 ng to about 500 ng, from about 100 ng to about 1,000 ng, from about 500 ng to about 5,000 ng, from about 500 ng to about 10,000 ng, from about 500 ng to about 25,000 ng, from about 500 ng to about 50,000 ng, from about 500 ng to about 100,000 ng, and from about 500 ng to about 150,000 ng of the iDNA molecule, wherein the iDNA molecule encodes a live-attenuated VEEV.

In certain preferred embodiments, the effective dose of the vaccine is selected from the group consisting of from about 1 ng to about 200,000 ng, from about 2 ng to about 150,000 ng, from about 3 ng to about 100,000 ng, from about 4 ng to about 90,00 ng, from about 5 ng to about 80,000 ng, from about 6 ng to about 70,000 ng, from about 7 ng to about 60,000 ng, from about 8 ng to about 50,000 ng, from about 9 ng to about 40,000 ng, from about 10 ng to about 30,000 ng, from about 10 ng to about 20,000ng, from about 10 ng to about 19,000ng, from about 10 ng to about 18,000 ng, from about 10 ng to about 17,000 ng, from about 10 ng to about 16,000 ng, from about 10 ng to about 15,000 ng, from about 10 ng to about 10,000 ng, from about 10 ng to about 5,000 ng, from about 10 ng to about 1,000 ng, from about 10 ng to about 500 ng, from about 100 ng to about 500 ng, from about 100 ng to about 1,000 ng, from about 500 ng to about 5,000 ng, from about 500 ng to about 10,000 ng, from about 500 ng to about 25,000 ng, from about 500 ng to about 50,000 ng, from about 500 ng to about 100,000 ng, and from about 500 ng to about 150,000 ng of the iRNA molecule, wherein the iRNA molecule encodes a live-attenuated VEEV.

In certain preferred embodiments, the effective dose has a concentration selected from the group consisting of from about 10³ PFU to 10⁷ PFU in 20 ul, from about 5×10³ PFU to 5×10⁶ PFU in 20 ul, from about 10⁴ PFU to 10⁶ PFU in 20 ul, from about 5×10⁴ PFU to 5×10⁵ PFU in 20 ul, from about 6×10⁴ PFU to 4×10⁵ PFU in 20 ul, from about 7×10⁴ PFU to 3×10⁵ PFU in 20 ul, from about 8×10⁴ PFU to 2×10⁵ PFU in 20 ul, from about 9×10⁴ PFU to 1×10⁵ PFU in 20 ul, and about 1×10⁵ PFU in 20 ul of the infectious genetic material.

In certain preferred embodiments, the effective dose is selected from the group consisting of from about 10 PFU to about 10,000 PFU, from about 10 PFU to about 5,000 PFU. from about 10 PFU to about 3,100 PFU, from about 10 PFU to about 3,000 PFU, from about 10 PFU to about 2,500 PFU, from about 50 PFU to about 5,000 PFU, from about 50 PFU to about 3,100 PFU, from about 75 PFU to about 2,500 PFU, from about 100 PFU to about 1,000 PFU, from about 200 PFU to about 800 PFU, from about 300 PFU to about 700 PFU, from about 400 PFU to about 600 PFU, from about 500 PFU of the infectious genetic material.

In certain preferred embodiments, an effective amount of a single dose of the vaccine is capable providing a therapeutic benefit to a subject in need thereof. In certain preferred embodiments, the vaccine has a low adverse reaction in a subject in need of the vaccine. In certain preferred embodiments, the infectious genetic material is used to prevent, reduce or substantially minimize a viral infection and/or prevent or diminish the symptoms of a viral infection. In certain preferred embodiments, the symptoms is selected from the group consisting of a fever, headache, rash, nausea, myalgia, arthralgia, respiratory failure, cardiovascular disease, hepatitis, cutaneous effects, central nervous system abnormalities, and combinations thereof. In certain preferred embodiments, the vaccine has a low adverse reaction in a subject in need of the vaccine. In certain preferred embodiments, the vaccine is administered by injection, including intramuscular injection and/or subcutis injection; by microinjection, including transdermal microinjection; and/or combinations thereof. the vaccine can also be administered by microneedles injection or needless administration.

Disclosed herein is an vaccine comprising the attenuated virus disclosed herein and a non-toxic pharmacological excipient thereof. In certain preferred embodiments, the pharmacological excipient comprise a preservative and/or a saline; and preferably the saline is a phosphate buffered solution. In certain preferred embodiments, the vaccine further comprises an adjuvant. In certain preferred embodiments, the vaccine is a multivalent vaccine. In certain preferred embodiments, the vaccine comprise an effective amount the attenuated virus.

In certain preferred embodiments, the effective dose has a concentration selected from the group consisting of from about 10³ PFU to 10⁷ PFU in 20 ul, from about 5×10³ PFU to 5×10⁶ PFU in 20 ul, from about 10⁴ PFU to 10⁶ PFU in 20 ul, from about 5×10⁴ PFU to 5×10⁵ PFU in 20 ul, from about 6×10⁴ PFU to 4×10⁵ PFU in 20 ul, from about 7×10⁴ PFU to 3×10⁵ PFU in 20 ul, from about 8×10⁴ PFU to 2×10⁵ PFU in 20 ul, from about 9×10⁴ PFU to 1×10⁵ PFU in 20 ul, and about 1×10⁵ PFU in 20 ul of the attenuated virus.

In certain preferred embodiments, the effective dose is selected from the group consisting of from about 1 PFU to about 100,000 PFU, from about 2 PFU to about 100,000 PFU, from about 5 PFU to about 50,000 PFU, from about 10 PFU to about 10,000 PFU, from about 10 PFU to about 5,000 PFU, from about 10 PFU to about 3,100 PFU, from about 10 PFU to about 3,000 PFU, from about 10 PFU to about 2,500 PFU, from about 50 PFU to about 5,000 PFU, from about 60 PFU to about 2,500 PFU, from about 70 PFU to about 1,500 PFU, from about 75 PFU to about 1,300 PFU, from about 80 PFU to about 1,200 PFU, from about 90 PFU to about 1,100 PFU, from about 100 PFU to about 1,000 PFU, from about 200 PFU to about 800 PFU, from about 300 PFU to about 700 PFU, from about 400 PFU to about 600 PFU, from about 500 PFU, from about 50,000 PFU to about 500,000 PFU, from about 10,000 PFU to about 100,000 PFU, from about 5,000 PFU to about 50,000 PFU, from about 2,500 PFU to about 25,000 PFU, and about 10,000 PFU of the attenuated virus.

In certain preferred embodiments, the effective dose is selected from the group consisting of from about from about 500,000 to about 5,000,000, from about 100,000 to about 1,000,000, from about 50,000 to about 500,000, from about 25,000 to about 250,000, and about 100,000 of the attenuated virus.

In certain preferred embodiments, an effective amount of a single dose of the vaccine is capable providing a therapeutic benefit to a subject in need thereof. In certain preferred embodiments, the vaccine has a low adverse reaction in a subject in need of the vaccine. In certain preferred embodiments, the infectious genetic material is used to prevent, reduce or substantially minimize a viral infection and/or prevent or diminish the symptoms of a viral infection. In certain preferred embodiments, the symptoms is selected from the group consisting of a fever, headache, rash, nausea, myalgia, arthralgia, respiratory failure, cardiovascular disease, hepatitis, cutaneous effects, central nervous system abnormalities, and/or combinations thereof. In certain preferred embodiments, the vaccine has a low adverse reaction in a subject in need of the vaccine. In certain preferred embodiments, the vaccine is administered by injection, including intramuscular injection and/or subcutis injection; by microinjection, including transdermal microinjection; and/or combinations thereof. In certain embodiment, the vaccine is administered by microneedles injection and/or needless administration.

Disclosed herein is a method of preventing and/or reducing and/or substantially minimizing a viral infection comprising directly administering an effective amount of the iRNA molecule disclosed herein, the iDNA molecule disclosed herein, the composition disclosed herein, the vehicle disclosed herein, the attenuated virus disclosed herein, the vaccine disclosed herein, and/or combinations thereof, in a subject in need thereof.

Disclosed herein is a method of preventing and/or reducing and/or substantially minimizing a viral infection comprising directly administering an effective amount of the iRNA molecule disclosed herein, the iDNA molecule disclosed herein, the composition disclosed herein, the vehicle disclosed herein, the attenuated virus disclosed herein, the vaccine disclosed herein, and/or combinations thereof, in a subject in need thereof.

Disclosed herein is a method of making the iDNA molecule disclosed herein comprising cloning a stably attenuated RNA virus into a plasmid.

Disclosed herein is a method of making the composition disclosed herein comprising mixing the iRNA disclosed herein with a pharmaceutically acceptable non-toxic component.

Disclosed herein is a method of making the vehicle disclosed herein, comprising mixing the iRNA disclosed herein, the iDNA molecule disclosed herein, the composition disclosed herein, and/or combinations thereof with a lipid and an emulsifier.

Disclosed herein is a method of making the virus disclosed herein, comprising transfecting an eukaryotic cell with the iDNA molecule disclosed herein.

Disclosed herein is a method of making the vaccine disclosed herein, comprising mixing the iRNA molecule of any one of the iRNA molecule disclosed herein, the iDNA molecule disclosed herein, the composition disclosed herein, the vehicle disclosed herein, the attenuated virus disclosed herein, and/or combinations thereof; and a non-toxic pharmacological excipient thereof.

Disclosed herein is a method of making a multivalent vaccine comprising mixing one or more the iRNA molecule disclosed herein, the iDNA molecule disclosed herein, the composition disclosed herein, the vehicle disclosed herein, the attenuated virus disclosed herein, and/or combinations thereof; and a non-toxic pharmacological excipient thereof; wherein the one or more live attenuated virus encoded in the iRNA and/or iDNA is different.

The disclosed vaccine, including the V5040 vaccine with rearranged V5040 viral genome is a safe and effective novel live attenuated vaccine for CHIKV. In certain embodiments, the use of CHIKV iDNA plasmid can be used to prepare novel live-attenuated CHIKV vaccine with rearranged RNA genome with improved safety profiles. In certain embodiments of the CHIKV vaccine, the genomic RNA was rearranged to encode capsid gene downstream from the glycoprotein genes. In certain embodiments, attenuated mutations derived from experimental CHIKV 181/25 vaccine were engineered into E2 gene of the CHIKV vaccine to improve safety profile. In certain embodiments, a DNA copy of rearranged CHIKV genomic RNA with attenuated mutations was cloned into iDNA plasmid pMG5040 downstream from the CMV promoter. After transfection in vitro, iDNA plasmids, such as pMG5040, efficiently launched replication-competent CHIKV virus, such as the V5040 virus, with rearranged genome and attenuating E2 mutations. In certain embodiments, the CHIKV vaccine, such as the vaccine with the V5040 virus, demonstrates that it is safe and immunogenic in murine models. Vaccination with the CHIKV vaccine disclosed herein, including subcutaneous administering the V5040 vaccine, elicited CHIKV-specific immune responses.

Disclosed herein is an infectious DNA (iDNA) plasmid capable of launching live-attenuated CHIKV vaccine in vivo. The disclosed vaccine, including the V5040 vaccine with rearranged V5040 viral genome is a safe and effective novel live attenuated vaccine for CHIKV. In certain embodiments, the use of CHIKV iDNA plasmid can be used to prepare novel live-attenuated CHIKV vaccine with rearranged RNA genome with improved safety profiles. In certain embodiments of the CHIKV vaccine, the genomic RNA was rearranged to encode capsid gene downstream from the glycoprotein genes. In certain embodiments, attenuated mutations derived from experimental CHIKV 181/25 vaccine were engineered into E2 gene of the CHIKV vaccine to improve safety profile. In certain embodiments, a DNA copy of rearranged CHIKV genomic RNA with attenuated mutations was cloned into iDNA plasmid pMG5040 downstream from the CMV promoter. After transfection in vitro, iDNA plasmids, such as pMG5040, efficiently launched replication-competent CHIKV virus, such as the V5040 virus, with rearranged genome and attenuating E2 mutations. In certain embodiments, the CHIKV vaccine, such as the vaccine with the V5040 virus, demonstrates is safe and immunogenic in murine models. Vaccination with the CHIKV vaccine disclosed herein, including subcutaneous administering the V5040 vaccine, elicited CHIKV-specific immune responses.

### DETAILED DESCRIPTION

### Interpretations and Definitions

Unless otherwise indicated, this description employs conventional chemical, biochemical, molecular biology, immunology and pharmacology methods and terms that have their ordinary meaning to persons of skill in this field (unless otherwise defined/described herein). All publications, references, patents and patent applications cited herein are hereby incorporated by reference in their entireties.

As used in this specification and the appended claims, the following general rules apply. Singular forms “a,” “an” and “the” include plural references unless the content clearly indicates otherwise. General nomenclature rules for organism classification also apply. That is order, family, genus and species names are italicized.

As used herein, the following terms shall have the specified meaning. The term “about” takes on its plain and ordinary meaning of “approximately” as a person of skill in the art would understand. The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%. The term “comprise,” “comprising,” “contain,” “containing.” “include,” “including,” “include but not limited to,” or “characterized by” is inclusive or open-ended and does not exclude additional, unrecited elements.

As used herein, the following terms shall have the specified meaning.

“attenuated” means with reduced virulence.

“infectious” means capable of self-replication.

“infectious genetic material” means an iDNA, iRNA and combinations thereof.

“iDNA” means infectious DNA. An infectious DNA encodes the genetic material of a virus that is capable of self-replication in a host cell.

“iRNA” means infectious RNA. An infectious RNA encodes the genetic material of a virus that is capable of self-replication in a host cell.

“V5040” means a live-attenuated vaccine virus V5040 prepared by using the pMG5040 plasmid.

“pMG5040” means a plasmid derived from iDNA plasmid p181/25. The pMG5040 was based on pUC backbone vector and encodes the full-length rearranged RNA genome of V5040 vaccine virus.

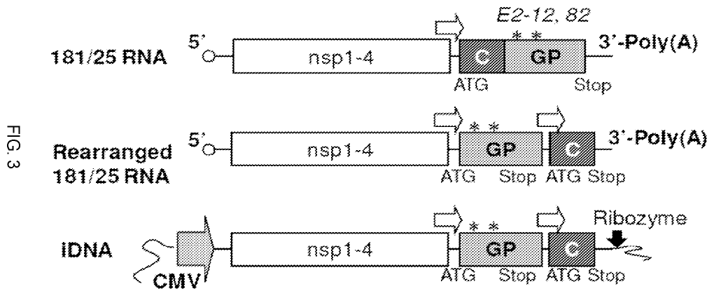

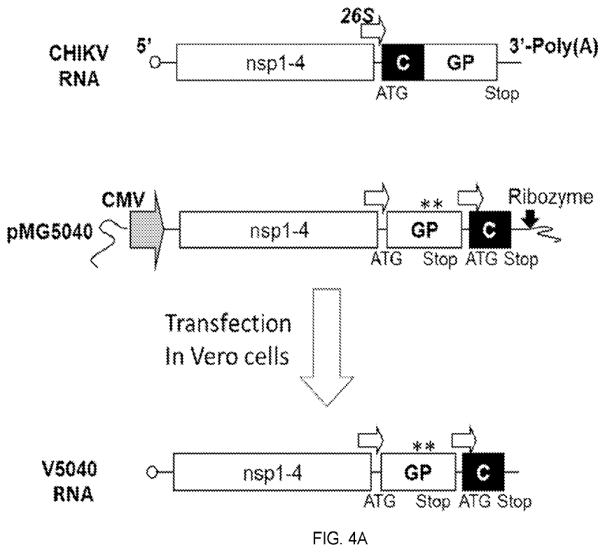

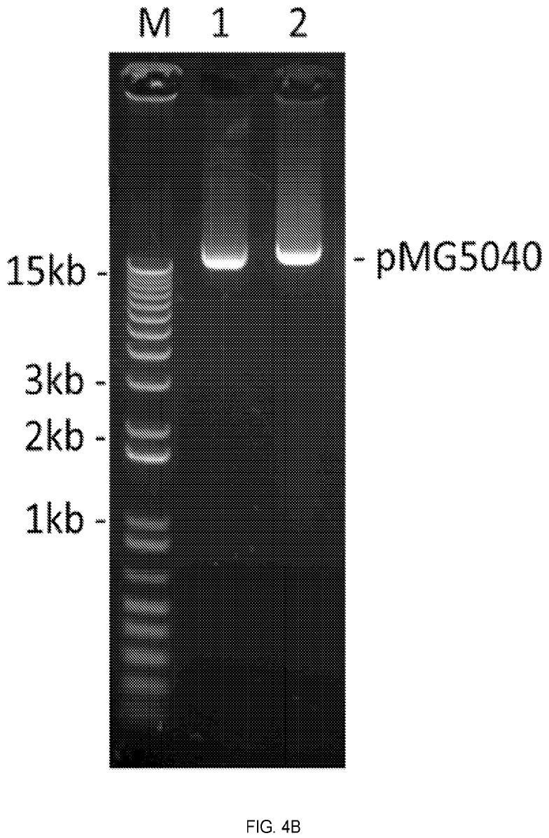

“rearranged” means the orders of all or part of the gene is rearranged. Example of rearrangement is found in FIG. 3 and FIG. 4A, and FIG. 12.

A “subject” is a vertebrate, such as a mammal, include, but are not limited to a human, primate, rodent, farm animal, sport animal (such as a horse) and pets. In certain embodiments, the subject is a human. In other embodiments, the methods find use in experimental animals (such as all species of monkeys), in veterinary application and/or in the development of animal models for disease. In certain embodiments, the vaccine is a VEE (such as TC-83) vaccine and the subject is a horse.

A “subject in need thereof” refers to any subject, patient, or individual who could benefit from the compound, composition, or methods described herein. A subject in need thereof of the present invention is preferably an animal, such as a mouse, ferret, chicken, pig, among others; and is preferably a mammal or bird; and most preferably a human.

A “therapeutically effective dose” or “pharmaceutically effective dose” or “therapeutically effective amount” or “pharmaceutically effective amount” means a dose or amount that produces the desired effect for which it is administered. The exact dose will depend on the purpose of the treatment, and will be ascertainable by one skilled in the art using known techniques.

The term “pharmaceutically acceptable” means approved by a regulatory agency of the federal or a state government or listed in the U.S. Pharmacopeia or other generally recognized pharmacopeia for use in animals, and more particularly, in humans.

The term “carrier” refers to a diluent, adjuvant, excipient, or vehicle with which the iDNA is administered. Such pharmaceutical carriers can be sterile liquids, such as water and oils, including those of petroleum, animal, vegetable or synthetic origin, such as peanut oil, soybean oil, mineral oil, sesame oil, combinations thereof and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, combinations thereof and the like. The composition, if desired, can also contain minor amounts of wetting or emulsifying agents, or pH buffering agents. These compositions can take the form of solutions, suspensions, emulsion, tablets, pills, capsules, powders, sustained-release formulations, combinations thereof and the like. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, and combinations thereof. Examples of suitable pharmaceutical carriers are described in “Remington's Pharmaceutical Sciences” by E. W. Martin.

A “pharmaceutically acceptable carrier” means, but is not limited to, a vehicle for containing the infectious genetic material that can be injected into a mammalian host without adverse effects. Suitable pharmaceutically acceptable carriers known in the art include, but are not limited to, gold particles, sterile water, saline, glucose, dextrose, or buffered solutions, combinations thereof and the like. Carriers may include auxiliary agents including, but not limited to, diluents, stabilizers (i.e., sugars and amino acids), preservatives, wetting agents, emulsifying agents, pH buffering agents, viscosity enhancing additives, colors, combinations thereof and the like.

A “long-lasting immunity” means that immunity elicited by the molecule, composition, vehicle, virus, and/or vaccine, protects the subject against a same or substantially similar virus for at least about 3 months, more preferably for at least about 6 months, more preferably for at least about 9 months, more preferably for at least about 12 months, more preferably for at least about 15 months, more preferably for at least about 18 months, more preferably for at least about 21 months, more preferably for at least about 24 months, more preferably for at least about 1 year, more preferably for at least about 3 years, more preferably for at least about 5 years, more preferably for at least about 10 years, more preferably for at least about 15 years, more preferably for at least about 20 years, more preferably for at least about 25 years, more preferably for at least about 30 years, more preferably for at least about 35 years, more preferably for at least about 40 years, more preferably for at least about 40 years, more preferably for at least about 50 years, more preferably for at least about 60 years, more preferably for at least about 70 years, more preferably for at least about 80 years, more preferably for at least about 90 years, and more preferably for at least about 100 years, during the life time of the subject

“CDC” means Center for Disease Control and Prevention.

“WHO” means World Health Organization.

“IM” means intramuscular, as directed to administration of vaccines.

“p.c.” means post-challenge, as directed to the period after the subject has been challenged with the target pathogen(s).

“NHP” means non-human primates.

“CMV” means the cytomegalovirus.

“CHIKV” means the Chikungunya fever virus, which is a mosquito-borne alphavirus that causes chikungunya fever.

“VEEV” means Venezuelan equine encephalitis virus.

“EEEV” means eastern equine encephalitis virus.

“SINV” means Sindbis virus.

“SFV” means Semliki Forest virus.

“YF” means yellow fever.

“YFV” means yellow fever virus.

“SIV” means simian immunodeficiency virus.

“HIV” means human immunodeficient virus.

“SRV” means simian type D retrovirus.

“STLV” means simian T-lymphotropic virus.

“WEEV” means western equine encephalitis virus.

“JEV” means Japanese encephalitis virus.

“ORF” means open reading frames.

“LNP” means lipid nanoparticles.

“IVT” means in vitro transcription.

“IFA” means immunofluorescense assay.

“PBS” means phosphate buffered saline.

“PRRs” means pattern-recognition receptors.

“TLR” means Toll-like receptors.

“RIG-I” means retinoic acid-inducible gene I.

“RLRs” means RIG-I-like receptors.

“NOD” means nucleotide-binding oligomerization domain.

“NLR” means NOD like receptor family protein.

“DOTAP” means cationic lipid 1,2-dioleoyl-3-timethylammonium-propane.

“IFN” means Interferons.

“CMI” means cell-mediated immunity.

“s.c.” means subcutaneous.

“FITC” means fluorescein isothiocyanate.

“PRNT” means plaque reduction neutralization assay.

“PRNT₅₀” means plaque reduction neutralization assay at 50%. This is the concentration of serum needed to reduce the number of plaques by 50% compared to the serum free virus.

“hCMV” means human CMV promoter.

“live attenuated” means the virus is capable of reproduction in a host cell and the virus has reduced virulence.

“vaccine” means a composition that reduces, prevents, substantially minimizes and/or substantially inhibit the infection of a virus, and/or reduces and/or prevents the symptoms caused by a virus. A vaccine achieves this by boosting the immune system of a subject against the virus. Examples of a vaccine include a DNA, a RNA, a liposomal, a viral (reproducing or non-reproducing), a replicon, a virus like particle, a protein, a portion of a protein. A multivalent vaccine is a vaccine that is capable of preventing, reducing, substantially minimizing and/or substantially inhibiting the infection of more than one different types of a virus, and/or capable of reducing and/or preventing the symptoms caused by more than one different types of a virus.

“GP” means a glycoprotein.

“nsp1-4” means non-structural proteins 1-4.

“181/25” means the virus or genetic material encoding the virus of attenuated vaccine candidate CHIK 181/25 (TSI-GSD-218), which was generated from CHIKV strain 15561 and had risks of reversion to a virulent form. The attenuation was a result from two point mutations.

“C” means the capsid protein.

“mcg” means microgram.

“PFU” means plaque-forming unit.

### iRNA Vaccines Against Positive-Strand RNA Viruses

Disclosed herein is a novel iRNA platform/system for vaccine use against positive-strand RNA viruses. In certain embodiments, the iRNA vaccine can be a self-replicating RNA encoding all proteins of the virus. In certain embodiments, the iRNA vaccine can encode an attenuated virus. In certain embodiments, the iRNA vaccine comprises rearranged genes, which can include an attenuating mutation that is resistant to reversion mutations. In some embodiments, the iRNA vaccine can be quickly synthesized synthetically from a known sequence of a pathogen, without the need for obtaining or manipulating pathogenic virus in order to attenuate it using classic methods. In some embodiments, the vaccine can offer protective immunity against one or more positive-strand RNA viruses in a single dose. In some embodiments, the iRNA vaccine is delivered via a DNA plasmid. In some embodiments, the iRNA vaccine is delivered by a nanoparticle, such as a lipid nanoparticle. In some embodiments, the iRNA vaccine is suitable for use in an outbreak and/or epidemic.

In some embodiments, the iRNA vaccine can be used for positive-strand RNA viruses in the realm Riboviria, including emerging viruses. In some embodiments, the iRNA vaccine can be used for positive-strand RNA viruses in the kingdom Orthornavirae. In some embodiments, the iRNA vaccine can be used for positive-strand RNA viruses in the phyla Kitrinoviricota, Lenarviricota, and/or Pisuviricota. In some embodiments, the iRNA vaccine can be used for positive-strand RNA viruses in the class Pisoniviricetes and/or Stelpavirictes.

In some embodiments, the iRNA vaccine can be used for positive-strand RNA viruses in the Togaviridae family.

In some embodiments, the iRNA vaccine can be used for positive-strand RNA viruses in the Alphavirus genus, such as western equine encephalitis virus (WEEV), Venezuelan equine encephalitis virus (VEEV), eastern equine encephalitis virus (EEEV), Sindbis virus (SINV), Semliki Forest virus (SFV), and Chikungunya virus (CHIKV).

In some embodiments, the iRNA vaccine can be used for positive-strand RNA viruses in the Flaviviridae family, such as Hepacivirus C.

In some embodiments, the iRNA vaccine can be used for positive-strand RNA viruses in the Flavivirus genus, such as, yellow fever (YF) virus, West Nile virus, Dengue virus, and Japanese encephalitis virus (JEV).

In some embodiments, the iRNA vaccine can be used for positive-strand RNA viruses in the Arenaviridae family, such as Lassa virus, Guanarito virus, Junin virus, Lujo virus, Machupo virus, Sabia virus, and Whitewater Arroyo virus.

In some embodiments, the iRNA vaccine can be used for positive-strand RNA viruses in the Coronaviridae family, such as MERS, SARS, and SARS-COV-2.

In some embodiments, the iRNA vaccine can be used for positive-strand RNA viruses in the Picornavirus family, such as rhinovirus (i.e. common cold virus).

In some embodiments, the iRNA vaccine can be used for positive-strand RNA viruses in the Retroviridae family, such as simian immunodeficiency virus (SIV), human immunodeficient virus (HIV), simian type D retrovirus (SRV), and/or simian T-lymphotropic virus (STLV).

In certain embodiments, the platform combines the strengths of an mRNA platform/system with the advantages of live-attenuated virus vaccines. For example, this platform allows for the manufacturing of vaccine in vitro, achievement of high efficacy, single-dose immunization, and reduction of the cost of the vaccine.

Efficient vaccination with genetic (DNA and mRNA) vaccines remains among the most important goals of current vaccinology. Although shown safe and effective in the clinic, the typical DNA and mRNA vaccines sometimes require large doses, multiple booster vaccinations, advanced adjuvants, complex delivery formulations, and/or specialized equipment such as in vivo electroporation.

From the vaccine antigen perspective, both DNA and mRNA vaccines express subunit antigens, which in most cases are poor immunogens, have low immunogenicity and require adjuvants for improved immunogenicity and efficacy. In an epidemic/pandemic scenario, multiple booster doses with DNA or mRNA vaccines increase the response time and result in higher morbidity and mortality. In addition, for mRNA vaccines, ultra-low cold chain complicates their use, especially in countries with limited ultra-cold chain ultrastructure.

This application describes a self-replicating “infectious” RNA (iRNA) platform/system that is based on iRNA encoding the whole live-attenuated virus. The vaccine would be suitable for positive-sense RNA viruses, including, but not limited to, alphaviruses such as western equine encephalitis virus (WEEV), Venezuelan virus (VEEV), eastern equine encephalitis virus (EEEV), Sindbis virus (SINV), Semliki Forest virus (SFV), and Chikungunya virus (CHIKV); flaviviruses such as yellow fever virus, West Nile virus, Dengue virus, and Japanese encephalitis virus, and Hepacivirus C; arenaviruses such as Lassa virus, Guanarito virus, Junin virus, Lujo virus, Machupo virus, Sabia virus, Whitewater Arroyo virus, combinations thereof and the like.

In some embodiments, attenuating mutations derived from known live-attenuated viruses, as well as attenuating strategies that prevent, reduce, substantially minimize and/or substantially inhibit the generation of a pathogenic virus are introduced to improve vaccine safety. In certain embodiments, the system/platform includes genetic rearrangement of viral genes within iRNA, which prevents, reduces, substantially minimizes or substantially inhibits the reversion to the wild-type genome of a pathogenic virus. In certain embodiments, the iRNA molecule is encoded in the DNA plasmid called iDNA, by placing the full-length iRNA under transcriptional control of a mammalian promoter, to improve thermal and genetic stability of iRNA. When such iDNA enters the cell, it synthesizes iRNA, which starts replication of live-attenuated vaccine virus. Live-attenuated vaccines represent about half of all approved vaccines, and FDA approved several live vaccines recently including against influenza, zoster and rotavirus confirming safety of this platform.

Live CHIKV vaccine would effectively contain outbreaks and save human lives. Live attenuated vaccines represent approximately half of all licensed vaccines in the U.S. Live vaccines Zostavax, RotaTeq (Merck), FluMist (AstraZeneca), Rotarix (GSK) have been recently approved showing that live attenuated platform can be configured to meet stringent FDA safety standards.

In some embodiments, the novel iRNA system/platform is based on the full-length, engineered genomic iRNA of one or more positive-strand RNA viruses. The iRNA encodes all the proteins needed for replication of the one or more viruses. In order to improve safety, iRNA of the one or more positive-strand RNA viruses can be engineered with rearranged genes, which represents an attenuating mutation that is resistant to reversion mutations. In certain embodiments, the iRNA is expressed in cells, starts replication of live-attenuated viruses, and represents an effective emergency vaccine against outbreaks and epidemics of positive-strand RNA viruses. In some embodiments, the iRNA vaccine can provide protective immunity with a single-dose vaccination. In some embodiments, the iRNA vaccine offers the potential advantages of synthetic manufacturing, as well as high purity, genetic stability, simplicity of production, single-dose vaccination, and/or long-lasting immunity.

### iRNA Chikungunya Vaccines

In some embodiments, the iRNA vaccine comprise a self-replicating RNA encoding all proteins of live-attenuated CHIKV virus.

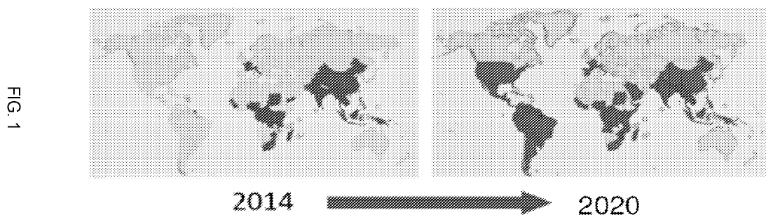

Chikungunya virus (CHIKV) is a mosquito-borne alphavirus that causes of chikungunya fever. CHIKV is a priority pathogen of CEPI and WHO. CHIKV causes wide-spread human infections and epidemics in Asia, Africa, and recently, in the Americas. CHIKV outbreaks are also spreading and continuing to spread across the world (FIG. 1) A vaccine is needed to contain outbreaks and prevent epidemics of CHIKV fever, however, there is no approved vaccine for CHIKV to date.

CHIKV is transmitted to humans primarily by Aedes aegypti mosquito. In addition, during the 2005-2006 epidemic in Reunion islands in the Indian Ocean that affected more than one-third of the island population and caused 284 deaths, a new mosquito vector A. albopictus, has been identified. Outbreaks of CHIKV included India in 2005-2006 with estimated 1.3 million people infected and the Americas since 2014, with more than 150,000 confirmed CHIKV infections in the Caribbean region only. CHIKV is also widespread in Africa and South East Asia. With an increase in global travel, the risk for rapid expansion of CHIKV to non-endemic areas has increased. Some travelers are viremic, and A. albopictus is common in urban areas of the U.S., raising concerns for the immunologically naïve population. Local transmission cases have been reported in Europe and the U.S. Changing climate patterns also favor geographical expansion of CHIKV. Given the current large outbreaks and the worldwide distribution of A. aegypti and A. albopictus, CHIKV represents a global public health threat.

CHIKV can cause fever, headache, rash, nausea, myalgia, and arthralgia. Complications include respiratory failure, cardiovascular disease, hepatitis, cutaneous effects, and central nervous system problems. More than 50% of patients who suffer from severe infection are over 65 years old, and more than 33% of them die. Current treatments for CHIKV are directed towards only symptoms of the disease and the treatments can include anti-inflammatory drugs, fluids, and bed rest. Antivirals and agents that restrict the cell-to-cell spread of the virus can be useful, but not FDA-approved. Arthralgia associated with the fever can persist for months or years and progresses to arthritis in some patients.

A CHIKV vaccine is needed to prevent the spread of CHIKV. However, currently, there is no approved CHIKV vaccine or specific antiviral therapy in the U.S.

For this situation, the CHIKV iRNA-based vaccine offers many advantages. Addressing the need for a safe and effective vaccine, we describe an iDNA and/or iRNA vaccine system to prevent CHIKV and associated diseases.

Preclincial studies of GMP lot of the novel iRNA CHIKV vaccine, yellow fever vaccine, Japanese encephalitis vaccine in relevant animal models, including non-human primates (NHP) show promising toxicity and immunogenicity profiles. The iRNA platform is suitable for different types of potentially epidemic or pandemic RNA viruses, including emerging viruses.

iRNA vaccines, when delivered via DNA plasmids can be a safe, immunogenic, and efficacious against infection with positive-strand RNA viruses for at least alphaviruses, flaviviruses, and arenaviruses. This DNA plasmid was sometimes called iDNA because it was designed to express iRNA. In the iDNA plasmid, iRNA is encoded downstream from RNA polymerase promoter, which drives synthesis of iRNA in the cells.

Here, describe a novel plasmid DNA that expressed the full-length genomic RNA of live-attenuated CHIKV from the eukaryotic promoter—a strategy termed iDNA. The iDNA plasmid can be transfected in cell culture to manufacture live-attenuated virus vaccine, or can be administered directly into patients' tissues to generate live-attenuated virus in vivo. In the latter case, iDNA plasmid is taken up by a limited number of cells, and CHIKV genomic RNA is transcribed in these cells. Then, CHIKV proteins are synthesized, genomic RNA is packaged into virus particle, and live-attenuated CHIKV is assembled and secreted from the cells. In immunocompetent BALB/c mice, the prototype 181/25 iDNA plasmid vaccine demonstrated safety and protective efficacy.

We also describe the use of CHIKV iDNA plasmid as a reverse genetics system to engineer a novel CHIKV vaccine. The V5040 CHIKV encoding a rearranged RNA genome was prepared to improve safety of live-attenuated CHIKV vaccine. Genetic rearrangements in many viruses are attenuating and highly resistant to reversions. However, it is unpredictable whether a rearrangement would negatively affect viral replication. Thus, until now it was not known if a genetic rearrangement of live attenuated CHIKV iDNA, iRNA, or viral vaccine can be made without impairing the infectivity and replication of the CHIKV. Here, we show, for the first time that an infectious genetic material (such as an iRNA and an iDNA) encoding a live attenuated CHIKV can be made and can cause efficient replication in mammalian cells.

We placed CHIKV capsid gene downstream of the glycoprotein gene under control of a duplicate subgenomic 26S promoter. In addition, two attenuating mutations derived from the prototype 181/25 vaccine were included in the E2 glycoprotein region. We discovered that live virus with rearranged genome replicates after pMG5040 plasmid is transfected in Vero cells. Thus, rearranged CHIKV iDNA pMG5040 launches live-attenuated CHIKV V5040 in Vero cell culture. Furthermore, vaccination of mice with V5040 confirmed its safety and elicited CHIKV-specific serum antibodies. Thus, we have developed a novel V5040 vaccine suitable for use as a vaccine for CHIKV.

Genetic rearrangements potentially can be utilized to engineer safer live-attenuated vaccines. Gene rearrangement is expected to be highly resistant to reversion because multiple independent mutations would be needed for reversion. Here, we applied genetic rearrangement for the development of novel CHIKV live-attenuated vaccine based on an investigational 181/25 live-attenuated CHIKV vaccine. In early studies, experimental CHIKV vaccine clone 181/25 was reported as highly immunogenic in Phase II clinical trial; however, mild transient arthralgia was observed in some patients. Vaccine was evaluated in 59 healthy volunteers, with 98% seroconversion rate. However, up to 8% experienced mild transient arthralgia. Reversion mutations have been detected in viremic patients that received the 181/25 IND vaccine indicating the need for safety improvement. Rearrangement can also represent an additional attenuating mutation.

We also describe a iRNA vaccine against a positive-strand RNA virus wherein the iRNA vaccine is delivered to the subject by a lipid nanoparticles (LNP), an iDNA plasmid in vivo delivery system, and combinations thereof and the like.

A chikungunya VLP-based vaccine and a live-attenuated measles vectored vaccine have been qualified to enter clinical phase II trials. Live-attenuated vaccines prepared by using reverse genetics have been described, and 181/25 Chikungunya vaccine has been tested in phase I/II clinical trials. Recently, Valneva tested live-attenuated Chikungunya vaccine in Phase III clinical trial. A promising therapeutic approach using DNA to launch a monoclonal antibody capable of neutralizing the virus was also reported. However, no CHIKV vaccine been approved to date. While investigational live-attenuated CHIKV vaccine developed by the US (clone 181/25) has been found to be highly immunogenic in a Phase II clinical trial; however, mild transient arthralgia was observed in some patients, demonstrating the need for safety improvement. To address the need for a safe and effective vaccine, described herein is a novel iRNA platform/system that is based on the full-length, engineered genomic iRNA of live-attenuated CHIKV. The iRNA encodes all the proteins needed for replication of a virus. In order to improve safety, CHIKV iRNA was engineered with rearranged genes, which represents an attenuating mutation that is resistant to reversion mutations. In certain embodiments, the iRNA is expressed in cells, starts replication of live-attenuated CHIKV, and represents an effective emergency vaccine against CHIKV outbreaks and epidemics, because of providing immunity with a single-dose vaccination. Previous vaccine candidates also suffer from high manufacturing costs, safety, and immunogenicity issues. A CHIKV iRNA vaccine as described herein offers the potential advantages of synthetic manufacturing, as well as high purity, genetic stability, simplicity of production, single-dose vaccination, and long-lasting immunity.

Early attempts of using attenuated CHIKV from a few point mutations were discontinued as a vaccine due to the severe side effects, most likely due to reversion of the attenuation. Others have since used reverse genetics to delete large portions of the CHIKV genome to reduce the likelihood that the attenuated virus will revert back to the more pathogenic virus. To accomplish this, 183 nucleotides from the CHIKV genome, located at the nsP3 gene and 6K gene are deleted. However, by deletion of a large portion of the nucleotides, which encodes 60 amino acids, is not ideal. For example, the nsP3 protein has important functions for the alphavirus replicase. As another example, large portion deletions of the C terminal hypervariable region of nsP3 affects interactions with several host proteins. Accordingly, there is a need for stable attenuating mutation in CHIKV that is resistant to reversal and would confer the full breadth of the CHIKV immunogen for a maximized immunogenicity profile and protection from the pathogenic CHIKV.

Several other experimental CHIKV vaccines have been described. These include live chimeric alphaviruses that carry CHIKV structural proteins, as well as formalin-inactivated vaccine adjuvanted with aluminum hydroxide. Furthermore, a virus-like particle (VLP) CHIKV vaccine protected nonhuman primates from CHIKV challenge and is currently in the clinical trials. DNA-based vaccines are promising vaccination strategies. However, many of experimental vaccines required two or more vaccinations to elicit protective immune response, which could be a disadvantage when rapid control of an outbreak is needed. A single-dose CHIKV vaccine that protects for the long-term can be a major benefit to global health. Measles virus-vectored CHIKV vaccine is currently in the clinical trials. Also, successful completion of the lot-to-lot Phase 3 trial of single-shot chikungunya vaccine candidate was announced, CHIKV Δ5nsP3 (VLA1553).

Applicant discovered a rearranged iDNA-derived V5040 vaccine designed to improve safety and induce effective immunity after a single injection. Live-attenuated vaccine has a favorable cost/benefit ratio and includes innovative safety features. It can be used to vaccinate individuals at risk of CHIKV infection, as well as for rapid deployment in outbreak situations to immunize against CHIKV in endemic and non-endemic areas.

Applicant has discovered a V5040 and pMG5040 based iRNA and iDNA CHIKV vaccines. In the proof-of-concept experiments, we showed that vaccination of BALB/C mice with the 181/25 vaccine protected them from CHIKV challenge. However, pMG5040 has not been tested in an iDNA format for vaccination. Advanced safety, immunogenicity, and efficacy studies in non-human primates can be used for pre-clinical evaluation of the CHIKV vaccine. Non-human primates have been successfully used to evaluate the safety and immunogenicity of live CHIKV and other vaccines. Another future project is to confirm that no reversion mutations occur in vivo. To address this, genetic stability studies in vivo (multiple passages in tissues and adult mouse brains) can be carried out to provide additional safety information as previously described.

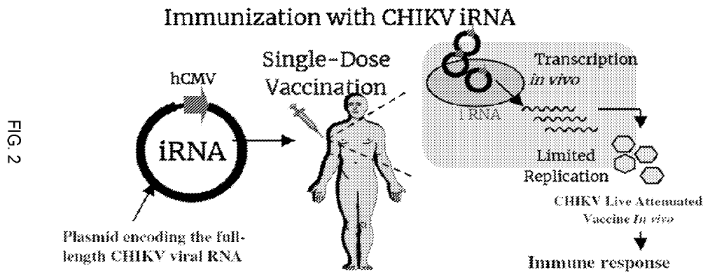

The iRNA platform is highly innovative. The iRNA can be delivered by either lipid nanoparticles (LNP) or by DNA plasmid (FIG. 2). The proposed iRNA approach represents a hybrid of live-attenuated, mRNA and DNA vaccines, whereby a vaccine plasmid encoding an iRNA is administered in vivo to initiate synthesis of a live attenuated virus. We previously demonstrated the feasibility of preparing iDNA-launched iRNA live virus vaccines for positive-sense RNA viruses and delivering them in vivo for safe and effective vaccination. Here, we describe a vaccine system/platform for emerging viruses with epidemic and pandemic potential. In some embodiments, the vaccine system/platform is an advanced synthetic-based iRNA technology. In certain embodiments, either iRNA or plasmids encoding iRNA virus vaccines can be quickly synthesized synthetically from a known sequence of a pathogen, without the need for obtaining or manipulating pathogenic virus in order to attenuate it using classic methods. Thus, iRNA vaccines, as described herein, can provide a transforming effect on the field of vaccinology. Our platform also provides a powerful tool to elucidate the contribution of specific mutations or genetic features to safety and immunogenicity. In particular, the plasmids can be blended for protection against several pathogens. The iRNA platform/system described herein can help to improve effective and rapid response to future epidemics by providing immune protection with a single-dose immunization.

Applicant has demonstrated the feasibility of iDNA approach to prepare CHIKV live-attenuated vaccines with rearranged genome. Applicant believes, iDNA vaccine can be used for vaccination directly, by injection into muscle of the vaccine recipient. Such iDNA vaccine could combine the benefits of conventional DNA immunization except it uses small quantities of DNA to launch efficacious live-attenuated vaccines. iDNA turns a small number of cells in muscle into cell-scale vaccine “factories”. The iDNA uses a well-established manufacturing technology of bacterial production of plasmids, which are easier to bank, control, and manipulate vs. live virus stocks. Since iDNA plasmids represent genetically-defined homogenous clones that after vaccination make the virus that undergoes minimal replication cycles, the probability of reversion mutations compared to traditional manufacturing is reduced. The iDNA vaccination can also have additional advantage for immunogenicity due to immunostimulatory effects of DNA vaccine. In addition to improved safety and immunogenicity, a CHIKV iDNA vaccine offers the potential advantages of high purity, genetic stability, simplicity of production, no cold chain, single-dose vaccination, and long-lasting immunity. Thus, iDNA could combine the advantages of DNA immunization and the high efficacy of live-attenuated vaccines.

### iRNA VEEV Vaccines

VEEV is a mosquito-borne alphavirus belonging to the Togaviridae family. VEEV causes human disease outbreaks and equine epizootics, mostly in the South, Central, and North America. Climate, ecological changes, and international travel increase the risk of VEEV reemergence. In addition, VEEV is a potential bioterrorism threat. Currently, there is no licensed human vaccine for VEEV, and the potential risks of VEEV outbreaks necessitate the development of a safe and effective vaccine.

Improvements are also provided for VEEV vaccines. Like CHIKV and other alphaviruses, because of the high rates of mutation in RNA viruses, there is a concern that live vaccines consist of a heterologous population and can have the risk of acquiring reversion mutations in the process of multiple passages during vaccine production, leading to regeneration of a more virulent virus. For example, safety considerations have hindered Food and Drug Administration (FDA) approval of TC83, a live-attenuated VEEV vaccine developed in the 1960s. The TC83 vaccine includes two attenuating mutations, 5′ A3 and E2-Arg120. Genetic reversions have been associated with the risk of adverse reactions. In contrast to a standard virus vaccine representing a population of viruses, an inherently stable iDNA plasmid represents a genetically-defined vaccine, which can provide many safety advantages.

The novel V4020 VEEV iDNA vaccine was engineered for improved safety, including additional attenuation strategies for preventing reversion mutations. The V4020 vaccine was prepared using an iDNA infectious clone that encodes the full-length rearranged genomic RNA downstream from the optimized CMV promoter. A recombinant plasmid pMG4020 encoding the genomic RNA of the V4020 vaccine virus has been confirmed to launch replication of a live-attenuated V4020 vaccine virus in vitro and in vivo. Safety, immunogenicity, and protection of V4020 virus against VEEV challenges were confirmed in BALB/c mice and non-human primates. A small dose of iDNA can be used to launch a live-attenuated virus. However, in order to launch V4020 virus in vivo, iDNA was previously delivered either using a transfection reagent or by electroporation, similar to traditional DNA vaccines.

In certain embodiments, the iDNA vaccine encoding live-attenuated VEEV can be efficiently delivered in vivo by a microneedle device using a single-dose vaccination with naked iDNA plasmid. For example, a pMG4020 plasmid can encode a live-attenuated V4020 vaccine of VEEV. The V4020 virus can contain structural gene rearrangement, as well as attenuating mutations genetically engineered to prevent reversion mutations. The pMG4020 can be administered in vivo to, for example, rabbits by injection. The injection can include the use of a hollow microstructured transdermal system (hMTS) microneedle device. In certain other embodiments, the iDNA and can be delivered using the MicronJet600 or the hMTS platform. In other embodiments, the iDNA and can be used delivered using the needle-free delivery systems. Following the administration, no adverse events to vaccination were observed. Moreover, animals that received pMG4020 plasmid have successfully seroconverted, with high plaque reduction neutralization test (PRNT) antibody titers, similar to those observed in animals that received V4020 virus in place of the pMG4020 iDNA plasmid. This supports that naked iDNA vaccine can be successfully delivered in vivo by using a single-dose vaccination with a microneedle device.

In certain embodiments, the iRNA-based vaccine can be useful as a live-attenuated, single-dose vaccine against a VEEV virus. The advantage is a single dose vaccine, efficient protection after a single dose, fully synthetic vaccine with low-cost of manufacture, storage and transportation. In certain embodiments, the iRNA-based vaccine can be useful against a VEEV virus and another positive-strand RNA virus in a single dose. In certain embodiments, the iRNA-based vaccine can be delivered via a DNA plasmid and/or a lipid carrier, such as a lipid nanoparticle.

### iRNA YFV Vaccines

Improvements are also provided for the YFV vaccines. In certain embodiments, the iRNA-based vaccine can be useful as a live-attenuated, single-dose vaccine against a YF virus. The advantage is a single dose vaccine, efficient protection after a single dose, fully synthetic vaccine with low-cost manufacture, storage and transportation. In certain embodiments, the iRNA-based vaccine can be useful against a YF virus and another positive-strand RNA virus in a single dose. In certain embodiments, the iRNA-based vaccine can be delivered via a DNA plasmid and/or a lipid carrier, such as a lipid nanoparticle.

### iRNA JEV Vaccines

Improvements are also provided for the JEV vaccines. In certain embodiments, the iRNA-based vaccine can be useful as a live-attenuated, single-dose vaccine against a JEV. The advantage is a single dose vaccine, efficient protection after a single dose, fully synthetic vaccine with low-cost manufacture, storage and transportation. In certain embodiments, the iRNA-based vaccine can be useful against a JEV virus and another positive-strand RNA virus in a single dose. In certain embodiments, the iRNA-based vaccine can be delivered via a DNA plasmid and/or a lipid carrier, such as a lipid nanoparticle.

### iRNA SARS-COV-2 Vaccines

Improvements are also provided to the SARS-COV-2 vaccines. In certain embodiments, the iRNA-based vaccine can be useful as a live-attenuated, single-dose vaccine against a SARS-COV-2 virus causing COVID-19 infections. The advantage is a single dose vaccine, efficient protection after a single dose, fully synthetic vaccine with low-cost manufacture, storage and transportation. In certain embodiments, the iRNA-based vaccine can be useful against a SARS-COV-2 virus and another positive-strand RNA virus in a single dose. In certain embodiments, the iRNA-based vaccine can be delivered via a DNA plasmid and/or a lipid carrier, such as a lipid nanoparticle.

### iRNA Multivalent Vaccines

Improvements are also provided to multivalent vaccines. In certain embodiments, the iRNA-based vaccine can be useful as a mixture of live-attenuated, single-dose vaccine against a multitude of viruses. The advantage is a single dose vaccine, efficient protection after a single dose, fully synthetic vaccine with low-cost manufacture, storage and transportation. In certain embodiments, the iRNA-based vaccine can be useful against a VEEV virus and another positive-strand RNA virus in a single dose. In certain embodiments, the multivalent iRNA-based vaccine can be delivered via a DNA plasmid and/or a lipid carrier, such as a lipid nanoparticle.

### EXAMPLES

### Example 1

### Generation and Immunogenicity of CHIKV iRNA Plasmid Vaccine

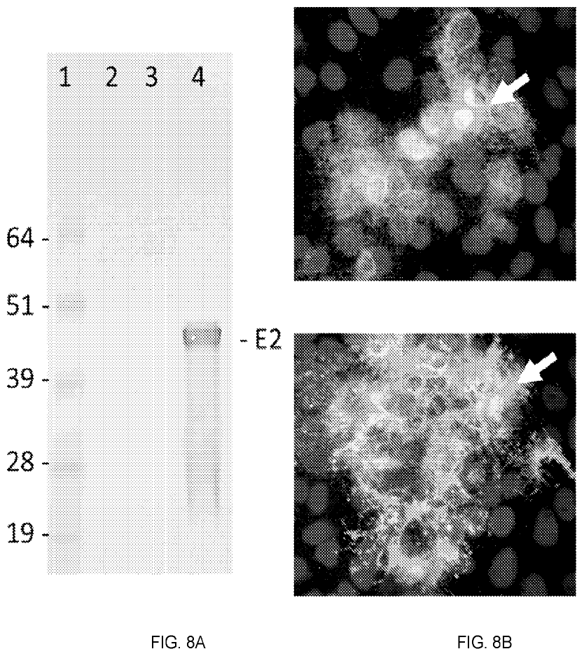

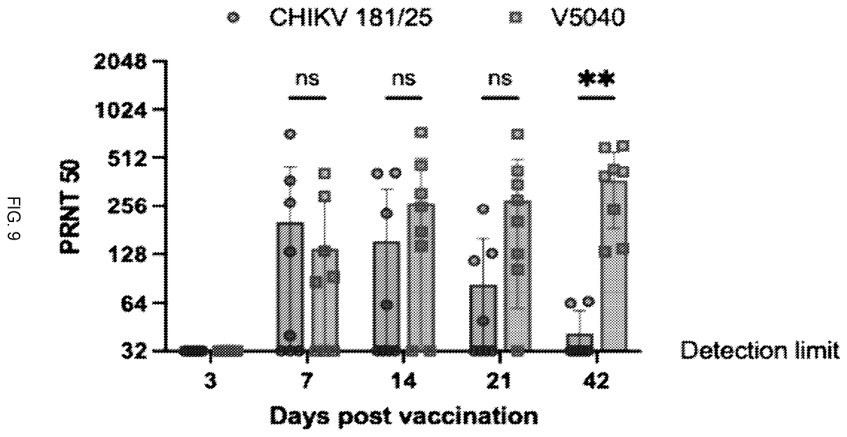

We prepared the 181/25 CHIKV iRNA vaccine prototype (FIG. 3). Because of its clinical history, the 181/25 vaccine is a good starting point for CHIKV vaccine development. This plasmid launched the CHIKV iRNA and a vaccine virus in vitro (FIG. 3 and FIG. 8A). We developed an iDNA approach as a novel infectious clone plasmid, in which the full-length viral RNA genome is transcribed from the CMV promoter. Using iDNA approach, we prepared the iDNA encoding the prototype 181/25 CHIKV vaccine. The latter encoded 181/25 genomic RNA in the plasmid downstream from CMV promoter. This plasmid launched the CHIKV 181/25-like vaccine virus in cell culture and in vivo. Vaccination of BALB/C mice with this 181/25 iDNA plasmid protected mice from CHIKV challenge. The attenuating mutations in the iDNA-generated virus were confirmed by DNA sequencing, with no reversions detected. Additionally, the iDNA-derived virus is expected to have lower heterogeneity (% SNPs) at the attenuating-mutation sites, E2-12 and E2-82, as compared to the prototype 181/25 virus. In a previous study, the iDNA-derived RNA had fewer SNPs vs. the 181/25 virus vaccine, suggesting potential safety advantage vs. classic live-attenuated virus.

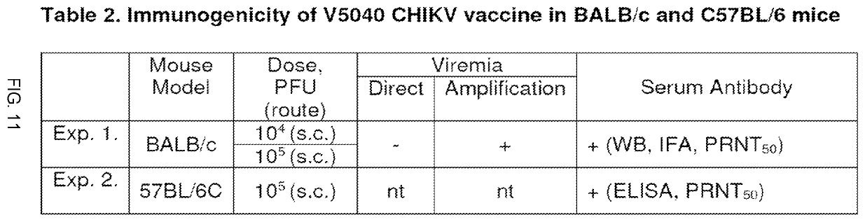

Also, vaccination of BALB/C mice with this prototype vaccine protected them from CHIKV challenge (FIG. 10, Table 1). The attenuating mutations in the iRNA-generated virus were confirmed by genotypic analysis and DNA sequencing, with no reversions detected.

Research involving mice was done according to approved institutional animal protocols. BALB/c mice (4-8 week-old, Noble Life Sciences, Woodbine, MD) were anesthetized with isoflurane prior to vaccinations. Mice (n=5 to 8 animals per group, female, two independent repeats) were vaccinated subcutaneously (s.c.) with the vaccine and control in the dorsal area at the doses 10⁴ and 10⁵ PFU. An exemplary vaccine is the V5040 vaccine virus and an exemplary control is the 181/25 vaccine. After vaccinations, animals were observed daily for signs of infection, morbidity and discomfort. Blood samples were collected from the retro-orbital sinus on days 0 (pre-bleed), day 2-4, and day 28. Viremia was evaluated on days 2-4 post-vaccination by either direct plaque assay, or by virus amplification in Vero cells followed by plaque assay. For virus amplification using co-cultivation, 20 μL of serum in 2 mL of complete medium was used to infect Vero cells in 75 cm² flask for 1 h, then 20 mL medium was added, and incubation was continued for 48 h. Supernatant was harvested, and the virus was assayed by plaque assay.

### Example 2

### Generation and Immunogenicity of VEEV iRNA Plasmid Vaccine

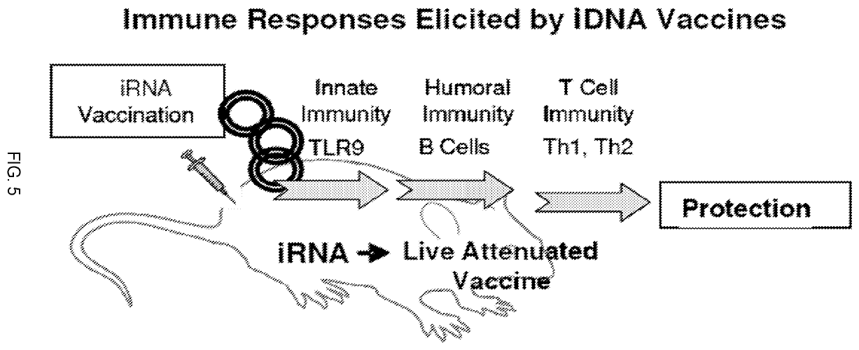

We also prepared an iDNA vaccine from the alphavirus TC83 VEEV (Venezuelan equine encephalitis virus). At 24 and 48 hours after transfection of cultured cells with this iDNA plasmid, some cells expressed TC83 antigens by immunofluorescence at 24 hrs, while the whole population expressed the antigens at 48 hrs (FIG. 3). Following injection of the TC83 iDNA into BALB/c mice, animals produced virus-neutralizing antibody (FIG. 5) and were protected from challenge. As expected, unrelated control did not provide protection. These results show the feasibility of iRNA vaccines delivered by plasmids.

In one example, we prepared a rearranged V4020 vaccine from the alphavirus vaccine TC83 for VEEV. We found that the V4020 vaccine was safe, immunogenic and efficacious in mice and non-human primates.

### Example 3

### Generation and Immunogenicity of a Next Generation, Further Attenuated CHIKV iRNA Vaccine by Genomic Rearrangement

One exemplary, next generation, iRNA CHIKV vaccine is based on the 181/25 investigational vaccine sequence. In immunocompetent BALB/c mice, this prototype vaccine showed safety and protective efficacy. Additionally, after transfection in Vero cells, the exemplary, next generation, CHIKV vaccine virus had fewer mutations as compared to the 181/25 virus vaccine, suggesting this approach is safer as compared to inoculation with live-attenuated virus. Since the IND 181/25 viral vaccine caused adverse reactions in early clinical trials (see Example 1), a strategy to improve safety is needed.

Our exemplary, next generation, CHIKV iRNA vaccine includes a genomic rearrangement as additional attenuating mutation. Genetic rearrangements in many viruses are attenuating and highly resistant to reversions. A genomic rearrangement of the iRNA-based vaccine (V5040) with delivery by LNP and/or iDNA can improve safety by (i) additional attenuation and/or (ii) preventing, reducing, and/or substantially minimizing and/or substantially inhibiting potential reversion.

To make the exemplary, next generation, V5040 live-attenuated CHIKV, we rearranged the structural genes and the 181/25 GP harboring E2 mutations. The genetic structures of the CHIKV pMG5040 and V5040 RNA are schematically shown in FIG. 4A. Compared to the wild type CHIKV genome, the structural genes of V5040 RNA genome were rearranged.

We designed the pMG5040 plasmid to launch replication of live-attenuated V5040 virus in mammalian cells. In the pMG5040 iDNA plasmid, the DNA copy of the genome of CHIKV vaccine virus is rearranged, with the capsid gene placed downstream from the glycoprotein genes using a duplicate subgenomic promoter. The rearrangement preserves the protein sequence and the immunogenic epitopes. Until now, it was not known if rearrangement can be engineered for CHIKV 181/25 live attenuated vaccine without impairing its replication. Therefore, in this study, we showed that V5040 virus efficiently replicates in pMG5040-transfected Vero cells. The rearranged V5040 virus was derived from pMG5040, and V5040 was evaluated for virus growth in cell culture. Finally, safety and immunogenicity was shown by vaccinating mice with V5040.

To prepare the live-attenuated vaccine virus V5040, in one example, we transfected Vero cells with pMG5040 plasmid. Vero cell line (American Type Culture Collection, Manassas, VA) was maintained in a humidified incubator at 37° C. in 5% CO₂ in αMEM supplemented with 10% fetal bovine serum (FBS) and gentamicin sulfate (10 μg/ml) (Life Technologies, Carlsbad, CA). Transfection was done by electroporation or other means. After transfection of Vero cells with the pMG5040, the CMV promoter directed transcription of the functional viral genomic RNA starting from the 5′ terminus to the 3′-terminal poly-A sequence.

In an exemplary embodiment, the Vero cells were transfected with 100 ng of pMG5040 and incubated at 37° C. The transfection medium was harvested at 48 h post-transfection, concentrated and partially purified by ultracentrifugation and resuspended in phosphate buffered saline (PBS), pH7.4. The V5040 titer was determined by a standard plaque assay in Vero cell monolayers in 6-well tissue culture plates using serial dilutions of the virus, and stained using neutral red. To detect the virus in samples with low titers (below the limit of direct plaque assay, 25 PFU/ml) co-cultivation of test samples with Vero cells was used to amplify the low viral load. The transfection medium was used to infect Vero cells at a multiplicity of infection of 0.01 to generate V5040 passage 1 (P1) virus. The P1 virus was harvested at 48 h post-infection (h.p.i.) and the titer of the V5040 P1 virus was determined. The virus was aliquoted and stored at −80° C. until used in vitro or in vivo.

To determine virus growth kinetics, Vero cells were infected in 75 cm² flasks with 100 PFU or 1000 PFU of V5040 virus, or CHIKV 181/25 control virus. The CHIKV 181/25 vaccine virus (control) was received from the World Reference Center for Emerging Viruses and Arboviruses (WRCEVA) through the University of Texas Medical Branch (UTMB) in Galveston, Texas. In the same virus growth kinetics experiment, Vero cells were also transfected with 100 ng of pMG5040 by electroporation. Samples of the growth medium were taken at 12 h intervals.

### Example 4

### Administration of the CHIKV iRNA Vaccine

In one example, the iRNA of the live-attenuated, engineered CHIKV vaccine V5040 can be administered as a single-dose formulation intramuscularly (IM). A single-dose format has the advantage of containing outbreaks and rapidly spreading epidemics, without the need for booster vaccinations, with the intention of being used as an emergency vaccine to stop spread of CHIKV epidemics worldwide. Initially, vaccine can target first responders, medical personnel, healthy adults, and travelers. When safety is confirmed, the vaccine can be considered for other populations including vaccinations in endemic areas, such as India, Asia and Central America.

### Example 5

### Genomically Rearranged Attenuated Non-CHIKV iRNA Vaccine

Genomically rearranged iRNA-based YFV and JEV vaccines can also be made and used using the principles disclosed herein, including the plasmids, vectors, cells, method of production and method of administration. For example, the capsid and envelop genes of the YFV gene can be rearranged to introduce stable attenuations as disclosed herein.

### Example 6

### Genomically Rearranged Attenuated Multivalent Vaccine

Genomically rearranged iRNA-based vaccines against multiple positive-strand RNA viruses can also be made as using full length attenuated RNA as disclosed herein.

Preparing the iRNA-based vaccine V5040 for CHIKV with attenuating genome rearrangement and compare CHIKV iRNA delivered in vitro and in vivo by LNP and iDNA. The genetic rearrangement places the capsid gene downstream the glycoprotein, which is an attenuating mutation in alphaviruses and can be highly resistant to reversion because multiple independent mutations would be needed to revert to the wild type CHIKV. The iRNA-derived V5040 can be initially examined for plaque phenotype, growth curve, and genetic stability (NGS) in vitro in Vero cells 3 and then compared side-by-side for safety, immunogenicity and efficacy in mice using either LNP and iDNA delivery.

The CHIKV iRNA is capable to launch live-attenuated V5040 CHIKV vaccine in vitro and in vivo. We showed that vaccination of BALB/C mice with the prototype CHIKV vaccine based on 181/25 protected mice from CHIKV challenge. When additional attenuating mutations, as well as strategies to prevent potential reversion mutations are used, adverse reactions previously detected using the prototype 181/25 vaccine can be reduced. The 181/25 vaccine is sourced from the World Reference Center for Emerging Viruses and Arboviruses (WRCEVA) through the University of Texas Medical Branch (UTMB) in Galveston, Texas. Genetic rearrangements in many viruses, including in alphaviruses, are highly resistant to reversions. Therefore, an attenuating rearrangement in V5040 iRNA is placed in the capsid gene downstream the glycoprotein genes (FIG. 3). The rearranged CHIKV iRNA can have improved safety, immunogenicity, and efficacy profiles with only a single IM vaccination using iRNA delivered by LNP and/or iDNA. As control, we can use the 181/25 IND vaccine virus. The rearranged CHIKV iRNA sequence can be confirmed by sequencing. Rearranged CHIKV iRNA can be prepared in the plasmid downstream from a T7 RNA polymerase. The CHIKV cDNA plasmid containing iRNA downstream from CMV promoter is available from the previous studies. Full-length DNA clones from CHIKV and VEEV, a related alphavirus can also be prepared.

### Example 7

### Plasmids Encoding Rearranged CHIKV iRNA V5040

Molecular biology. The capsid (C) gene is cloned downstream from the glycoprotein using the duplicate subgenomic promoter. For example, the structural gene region can be split into two open reading frames (ORFs), one expressing GP genes E3-E2-6K-E1, and the other expressing the C gene only (see FIG. 3). Each structural ORF is expressed from its own 26S subgenomic promoter and includes the translational start and stop codons. The genes are rearranged, with the GP genes placed in front of the C gene in the CHIKV plasmid. The full-length DNA copy of the engineered, rearranged genomic RNA can be cloned into a DNA vector plasmid containing the T7 bacteriophage promoter for production of iRNA in vitro. Additionally, CHIKV iRNA is cloned downstream from the CMV promoter for transcription of the rearranged genomic RNA in mammalian cells.

The rearrangement preserves the protein sequence and thus the immunogenic epitopes. The rearranged vaccine, V5040 iRNA can generate clonally purified progeny virus and homogeneous virus that is well-characterized, attenuated, and have a unique genetic signature. Such a homogenous population virus is can have a safety advantage over the mixed-population classic live-attenuated vaccine, which consists of heterogeneous sub-populations (quasi-species). Furthermore, similarly to other viruses, the rearrangement can be attenuating and resistant to reversion because many independent mutations would be needed to revert to the wild-type sequence.

As another example, the CHIKV structural gene region was split into two open reading frames (ORFs), one expressing GP genes E3-E2-6K-E1, and the other expressing the C gene only. Each structural ORF was expressed from its own 26S subgenomic promoter, and included translational start and stop codons. The C and GP genes were rearranged in the V5040, with the GP genes placed in front of the C gene (FIG. 4A). Artificially rearranged genomes lead to the attenuation of many viruses and are resistant to reversions because many independent mutations would be required to restore the wild type virus sequence.

The full-length, functional RNA genome of the V5040 vaccine virus was encoded in the pMG5040 plasmid downstream from the optimized CMV promoter. The pMG5040 was prepared by re-designing the prototype CHIKV iDNA clone. For example, the pMG5040 plasmid was derived from iDNA plasmid p181/25. The pMG5040 was based on pUC backbone vector and encoded the full-length rearranged RNA genome of V5040 vaccine virus (FIG. 4A). The capsid (C) gene was cloned downstream from the glycoprotein gene by overlapping PCR and expressed using the duplicate subgenomic promoter. The ATG codon was introduced at the 5′ of E3 gene within the glycoprotein (GP) genes, while a TGA stop codon was introduced at the 3′ of the C gene. The full-length genomic cDNA of V5040 virus was placed in the pMG5040 under transcriptional control of the optimized CMV promoter. The hepatitis delta ribozyme was introduced downstream from the V5040 cDNA to ensure cleavage of the genomic RNA transcript after the synthetic poly (A) at the viral 3′ end. The cDNA of V5040 also maintained attenuating mutations Thr12Ile and Gly82Arg (both in the E2 gene) derived from the 181/25 prototype vaccine sequence.

The iDNA plasmid pMG5040 containing the rearranged, full-length CHIKV iDNA was isolated from E. coli. The CHIKV sequence was confirmed by DNA sequencing. Bioinformatic analysis was performed to ensure that rearranged CHIKV sequences do not interfere with plasmid production in E. coli or the translation of the viral genome in eukaryotic cells. We confirmed the presence of the authentic ORFs and the absence of any strong internal bacterial promoter and transcription sites that can lead to expression of potentially toxic proteins and inhibit plasmid growth in E. coli. The ORFs were predicted by using NCBI ORF Finder software. Potential bacterial promoters were predicted by using BPROM software, which is a bacterial promoter recognition program with approximately 80% accuracy and specificity. In addition, rearranged sequence was screened for potential splice sites that can lead to degradation of RNA in the nucleus, by using software developed within Berkeley Drosophila Genome Project. The amino acid sequence of CHIKV proteins was kept according to GenBank 181/25 TSI-GSD-218 CHIKV 181/25 vaccine #L37661 except additional Met at the start of GP genes, and a stop codon at the end of C gene (FIG. 4A).

The resulting plasmid pMG5040 was propagated in E. coli Stbl3 cells (Thermo, Carlsbad, CA) using standard Luria Broth (LB) medium in the presence of kanamycin. Plasmid was isolated by an endotoxin-free DNA isolation method (Qiagen,

Valencia, CA), or a similar DNA isolation method, according to manufacturer's instructions. Finally, pMG5040 was formulated in phosphate-buffered saline (PBS) to a concentration of ˜1 mg/ml. This process resulted in a transfection-grade, sterile DNA with 95% supercoiled DNA and an A260/A280 ratio of ˜1.9, as well as minimal residual endotoxin, RNA, genomic DNA, and protein impurities.

### Example 8

### Evaluation of iRNA-Generated Viruses In Vitro

The iRNA can be generated in vitro using pT7-V5040 as a template for run-off transcription in vitro using the T7 polymerase. DOTAP lipid-based LNP can be used to encapsulate iRNA for vaccination. The iDNA pCMV-V5040 can be formulated in PBS for vaccination. The 181/25 vaccine virus can be used as control. Vero (ATCC CCL-81.5) cells can be (1) transfected with iRNA-LNP, (2) transfected with iDNA using Fugene transfection reagent, or (3) infected with control 181/25 CHIKV. Vero cells in serum-free medium are often used in the vaccine manufacturing and testing. Samples of live attenuated viruses can be collected from culture medium at 6 hr intervals for 96 hr. The rearranged viruses harvested from iRNA- or iDNA-transfected Vero cells can be characterized for plaque phenotype, growth curve, and genetic stability (by Illumina NGS). The control can be standard IND 181/25 viral vaccine. The genetic and phenotypic features of the three viruses can include: kinetic parameters of replication in tissue culture, antigenic properties, genetic stability, and molecular heterogeneity by the following in vitro assays: (1) plaque assay and phenotype; (2) virus growth curves; (3) western blot; (4) immunofluorescence; and (5) NGS sequencing of each virus including 181/25 control to assess population heterogeneity and genetic stability in mammalian cells.

For example, CHIKV-specific antibody response was determined using sera collected on day 28 post vaccination by Western blot and indirect immunofluorescence assay (IFA). For example, mouse serum was probed in western blot with the lysates of CHIKV 181/25 infected Vero cells at 1:100 dilution, followed by alkaline phosphatase (AP)-conjugated goat anti-mouse IgG (H+L) secondary antibody (1:1000 dilution). As another example, IFA, Vero cells seeded in chamber slides were infected with V5040 at MOI 0.01. At 24 h post-infection, Vero monolayers developed foci of V5040-infected cells. Monolayers were fixed with acetone and probed with mouse serum diluted 1:25, followed by Fluorescein isothiocyanate (FITC)-conjugated goat anti-mouse IgG (H+L) secondary antibody (1:25 dilution). Nuclei were stained with VectaShied mounting medium containing propidium iodide (Vector Laboratories, Inc., Newark, CA).

### Example 9

### Vaccination with CHIKV V5040 iRNA Vaccines In Vivo

Mice can be vaccinated with (1) LNP-formulated iRNA, (2) iDNA-formulated iRNA, or (3) 181/25 virus. Based on the clinical and pathologic similarities with CHIKV infection in humans, C57BL/6 and CD-1 mice offer useful and realistic models for studying CHIKV infection and vaccination.

We can optimize and evaluate iRNA vaccination in 14-day-old male and female C57BL/6 mice (Harlan Sprague-Dawley, Indianapolis, IN). The iRNA is an innovative technology to launch live attenuated virus. The best method of iRNA delivery in vivo can be determined. The iRNA delivery can be optimized in C57BL/6 mice (groups of 8, male and female) using a single-dose vaccination with 50 μg of each vaccine (1) LNP-formulated iRNA, (2) iDNA-formulated iRNA using in vivo transfection reagent (In vivo-jetPEI, PolyPlus, Illkirch, France), (3) control 181/25 virus. The proposed dose of 50 μg is similar to mRNA vaccines. The iRNA vaccine effects can be measured in vivo by evaluating viremia after vaccination, as well as by the magnitude of the immune response to CHIKV antigens as evaluated by the analysis of CHIKV-neutralizing antibody (plaque reduction neutralization assay, PRNT) and cell-mediated responses (ELISPOT). Animals can be challenged with neurovirulent CHIKV to demonstrate protection of proposed V5040 CHIKV iRNA vaccine as we described elsewhere. Groups of 8 animals can provide sufficient statistical power to generate valid results.

For example, in a Plaque Reduction neutralization assay (PRNT50), C57BL/6J mice (5-6 week-old, Jackson Laboratories) were vaccinated with test vaccines (105 PFU/mouse) or PBS as described above. Animals (n=8/group equal number of sex) were sacrificed at various time points (3, 7, 14, 21, and 41 days post-vaccination) and the total blood was collected by terminal bleeding. For PRNT, diluted homologous virus was mixed with the same volume of serially diluted sera (1:64 to 1:2048, two-fold) and incubated at 37° C. for one hour. Virus-serum mixture was added to Vero 76 cells confluently grown in 24-well plates and incubated at 37° C. for 1 h for virus adsorption. Cells were washed with PBS once, and overlayed with an overlay medium (1×EMEM with 2% FBS, 0.75% methylcellulose). After four days of incubation in a CO₂ incubator at 37° C., cells were fixed and viral foci were visualized with a crystal violet staining solution (2% paraformaldehyde, 10% ethanol, and 1% crystal violet). Number of viral foci forming units were normalized based on the average from the mock group and PRNT50 was calculated with a dose-response analysis with a four-parameter logistic model (XLfit, IBDS, UK). Statistical analysis was performed with GraphPad Prism (9.4.1).

Preparation and evaluation of iRNA-based vaccines and comparison LNP- and iDNA-methods of iRNA delivery in vitro and in vivo. The V5040 virus can replicate at a slower rate than the classic 181/25 virus similarly to other rearranged alphaviruses. Additionally, the iRNA-derived V5040 virus can have high homogeneity (% single nucleotide polymorphism, SNPs), as compared to its 181/25 vaccine. No reversion of rearranged sequence to the wild type sequence is detected. The method for iRNA delivery in vivo (LNP or iDNA) can be compared. LNP-formulated iRNA can launch the vaccine directly in the cytoplasm, while iDNA-formulated iRNA requires nucleus machinery to synthesize iRNA and transport to the cytoplasm via nuclear pores. However, LNP-iRNA can require ultra-cold storage/transportation, while iDNA can be formulated to store at ambient temperature. When can deliver, via LNP or iDNA, the iRNA is can activate innate immunity via TLR mechanism and efficiently prime specific immune response to CHIKV vaccine.

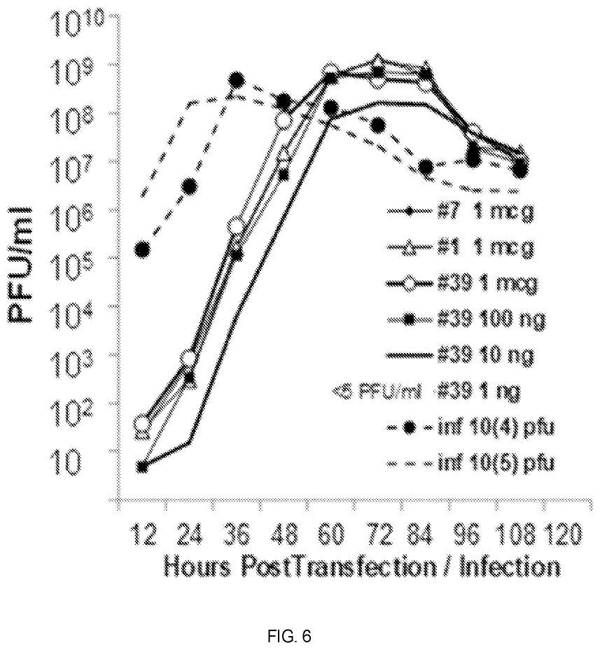

CHIKV is a cytoplasmic virus, and the genomic RNA replicates exclusively in the cytoplasm. Similarly, to the 181/25 control virus, the LNP-formulated iRNA is also can launch replication of V5040 in the cytoplasm. In contrast, iDNA-delivered iRNA must be introduced in the nucleus for synthesis of iRNA. The iRNA V5040 can work to launch from LNP-or iDNA-formulated iRNA because only a few copies of intact iRNA in the cytoplasm are needed to initiate replication of live attenuated CHIKV. When LNP or iDNA vaccination results in low immunogenicity adjuvants and/or microneedles can be added to enhance immunogenicity of iRNA vaccine. The iDNA strategy for (+) strand RNA viruses can be a viable approach (FIG. 6). FIG. 6 shows that only 10 ng of iDNA-formulated iRNA is sufficient to launch replication of CHIKV.

### Example 10

### Evaluate Platform Applicability of iRNA Technology for JEV and YFV and Multivalency in Small Animals

iRNA platform and in vivo delivery method developed in Examples above for applicability to other emerging virus pathogens with epidemic potential, such as YFV and JEV. YFV iRNA vaccine based on live-attenuated vaccine 17D can be prepared. Similarly, JEV iRNA vaccine can be prepared by using JEV vaccine SA-14-14-2. The three iRNA vaccines can be blended as a multivalent iRNA vaccine.

48 animals (C57BL/6, equal number of males and females) can be divided into 4 groups (12 mice per group) and vaccinated via best method (see Aim 1) with 50 ug of each iRNA vaccine: (1) V5040 CHIKV iRNA; (2) JEV iRNA; (3) YFV iRNA, and (4) blended CHIKV, YFV, JEV IRNAs. Each iRNA formulation can be delivered using the best iRNA delivery method as described above. The dose chosen is close to current mRNA vaccines. Control animals (group 5) can be injected with 105 PFU of 181/25 virus vaccine. Blood samples can be taken every 2-7 days for viremia, NGS, and serology. NGS of the virus from viremic mice can compare genetic variability and determine potential reversion rates of the vaccines. Immunogenicity can be determined by analysing neutralizing immune response (PRNT) and CMI (ELISPOT) to respective viruses. Statistical analysis can be performed using Student t-test.

### Example 11

### Safety, Immunogenicity and Efficacy Studies of iRNA-Based CHIKV Vaccine

We can also evaluate the safety, immunogenicity and efficacy studies of iRNA-based CHIKV vaccine in relevant animal models including CD-1 mice and cynomolgus macaque non-human primates. In the previous Phase II clinical trial, the CHIKV 181/25 vaccine was highly immunogenic (98% seroconversion rate) and well-tolerated. However, up to 8% of those receiving the vaccine had mild transient adverse reactions, suggesting the need for safer CHIKV vaccine. The CHIKV V5040 vaccine based on iRNA containing genetic rearrangement to attenuate and secure safety profile of the vaccine can address this need. Importantly, rearrangements are not only attenuating but also resistant to reversion. Reversion mutations have been detected in viremic patients that received the classic 181/25 IND vaccine. Therefore, proposed rearrangement and iRNA approach would improve two potential safety issues: (i) improve CHIKV vaccine safety by additional attenuation, as described for other viruses with rearranged genomes 9, (ii) prevent unexpected reversions to virulent genotype, and (iii) improve immunogenicity by stimulating innate immune response via iRNA self-adjuvant properties.

Even with a highly attenuating rearrangement, immunogenicity and protective immune responses of CHIKV vaccine can be improved by using iRNA technology. Unlike live viral vaccines, the iRNA vaccine can be self-adjuvanting and stimulate innate immune response before vaccine particles are launched (FIG. 5). Thus, similarly to mRNA vaccines, after iRNA vaccine administration, the iRNA can be recognized by multiple pattern-recognition receptors (PRRs), such as Toll-like receptors (TLRs), retinoic acid-inducible gene I (RIG-I)-like receptors (RLRs), and the nucleotide-binding oligomerization domain (NOD)-like receptor family proteins (NLRs), for the production of IFNs and proinflammatory cytokines, thus providing enhanced antigen-presenting capability 17 when CHIKV vaccine antigens are generated.

### Example 12

### Vaccination and Challenge with CHIKV V5040 iRNA Vaccines in Outbred CD-1 Mice

CD-1 mouse model can be used to test safety, immunogenicity and efficacy of iRNA CHIKV V5040 vaccine in a dose escalation study to determine optimal dose of iRNA vaccine. Briefly, forty-eight outbred CD-1 mice (12 mice/group, male and female) can be vaccinated using iRNA as described in herein.

The groups are the following: (i) V5040 iRNA intramuscularly 0.5 μg dose, (i) v5040 iDNA 5 μg dose; (iii) v5040 iDNA 50 μg dose; (iv) control group sham-vaccinated. The upper dose of 50 μg is chosen due to current mRNA vaccines.

Blood samples can be taken every 2-7 days for viremia, NGS, and serology. Immunogenicity can be determined by analysing innate immunity, neutralizing immune response (PRNT) and CMI (ELISPO). Mice can be challenged s.c. (d28) with CHIKV at a dose of 1×10⁵ PFU in 20 μl as described for this animal model. CD-1 mice challenged s.c. with CHIKV develop lethargy, difficulty walking, dragging of hind limbs, and reduced weight gain within 7-10 days after infection. During the initial 6-7 days, the animals display viremia and high levels (10⁸-10⁸ PFU/mL) of CHIKV in leg muscle. After challenge, animals can be observed daily for clinical signs of infection and body weight. Morbidity and viremia can be recorded, and statistical analysis can be performed.

### Example 13

### Safety, Immunogenicity and Efficacy of CHIKV V5040 iRNA Vaccine in NHP

Cynomolgus macaques (Macaca fascicularis) may represent an ideal model to assess the safety of CHIKV vaccines. In prior research, multiple parameters have been measured during NHP CHIKV infection to assess the effectiveness of potential vaccines at hindering the development of CHIKV infections.

Cynomolgus macaques of similar age (>3 yr) and weighing 3-6 kg, free of simian immunodeficiency virus (SIV), simian type D retrovirus (SRV), simian T-lymphotropic virus (STLV), and alphavirus antibodies against western equine encephalitis virus (WEEV), Venezuelan virus (VEEV), eastern equine encephalitis virus (EEEV), Sindbis virus (SINV), Semliki Forest virus (SFV), and CHIKV (assayed by hemagglutination inhibition), can be used. The study can be approved by the Institutional Animal Care and Use Committee, and all animals were handled in accordance with guidance from the American Association for Accreditation of Laboratory Animal Care.

CHIKV V5040 iRNA vaccine can be randomly assigned to macaque cohorts; one (n=4) can be vaccinated intramuscularly with iRNA with best formulation and dose (see Aims 1 and 2), and a second (n=4) can be vaccinated with the control vaccine 181/25. A third cohort (n=7) can be sham-vaccinated intramuscularly with saline. Anesthetized animals can be vaccinated either intramuscularly in the upper deltoid, with a single inoculation of either iRNA in saline or 5.0 log10 PFU of control vaccine in a volume of 100 μL. The animals can be observed for signs of any clinically recognizable adverse responses and bled on days 1-3, 15, and 50 after vaccination. Serum samples can be collected. Immunogenicity can be determined by analysing neutralizing immune response (PRNT) and CMI (ELISPOT). Statistical analysis can be performed using pre-bleed sera as control. Cross-reactivity of the antiserum can be examined by assessing neutralization of CHIKV strains available at UTMB and/or via CEPI network.

On day 52 after vaccination, anesthetized macaques can be challenged with a single subcutaneous inoculation in the upper deltoid of WT CHIKV-LR (5.0 log10 PFU in a volume of 100 μL), as described elsewhere. Blood can be collected on days 1-3, 6, 9, 13, and 35 after challenge, when the experiment can be terminated and necropsies performed. Morbidity and mortality can be determined, and statistical analysis performed using Student's t test. Tissues can be placed in 10% zinc-formalin for histopathological analysis. Some tissues can be also placed in RNAlater solution and frozen for viral titration by plaque assay: axillary, bronchial, and inguinal lymph nodes.

The use of two mouse models (C57BL/6 and outbred CD-1) and an NHP model can add rigor and generate statistically valid results. The iRNA vaccine can be safe and induce significant, broadly cross-neutralizing immune response due to the immunostimulatory effects of iRNA due to activation of innate immunity. In addition to improved safety and immunogenicity, rearranged V5040 CHIKV iRNA has an advantage in manufacturing, storage, genetic stability, and transportation with no need for a cold chain with the iDNA formulation.

### Example 14

### Chemistry, Manufacturing, and Control (CMC) Development

Manufacturing process and analytical methods: Production of iRNA is done in vitro using in vitro transcription (IVT) reaction. Formulation with LNP is described below. Formulation of iRNA within iDNA is achieved by cloning iRNA downstream from the CMV promoter to transcribe iRNA in vivo. Experiments showed feasibility of iRNA-based vaccines in small animals. Advanced development including testing an NHP, development of manufacturing process, as well as GMP manufacturing, release testing and IND-enabling preclinical studies can be done according to industry protocols.

The iRNA vaccine can be prepared by using T7 RNA polymerase transcription in a GMP facility to prepare GMP-grade vaccines for Phase 1-2 clinical trials according to FDA and ICH regulations. After Quality Control tests, iRNA can be formulated with LNP. To promote effective delivery of iRNA in vivo, we complex the iRNA with PEGylated and cationic lipids to form LNPs. For that purpose, an ethanol dialysis approach can be used. LNPs will include cationic lipid 1,2-dioleoyl-3-timethylammonium-propane (DOTAP). Lipids in ethanol can be combined with RNA in citrate buffer and emulsified, followed by dialysis to remove ethanol and promote LNP-iRNA self-assembly. At these conditions, the LNPs encapsulate ˜95% of RNA. The LNPs represent largely unilamellar vesicles with a mean diameter of 75 nm as determined by light scattering and cryoelectron microscopy. Assuming a uniform population of LNPs with this size, this is can contain ˜1 iRNA molecule per particle.

iDNA formulation represents plasmid encoding iRNA downstream from the CMV promoter. Manufacturing of plasmid iDNA from E. coli can be done using established methods for production of the bacterial cell bank, fermentation, harvest/lysis of the biomass, and downstream DNA purification. This process results in a sterile DNA product with 95% supercoiled DNA and an A260/A280 ratio of ˜1.9, as well as minimal residual endotoxin, RNA, genomic DNA, and protein impurities. Quality control can include (i) endotoxin testing, (ii) agarose gel, (iii) SDS-PAGE; (iv) restriction enzyme analysis and (v) DNA sequencing.

Storage requirements for final product: LNP-formulated iRNA vaccine can be formulated by lyophilization for long-term storage at refrigerated temperature. The mRNA-LNPs can be lyophilized, and the physicochemical properties of the lyophilized material do not significantly change for 12 weeks after storage at room temperature and for at least 24 weeks after storage at 4° C.

As any DNA vaccine, the iDNA-formulated iRNA vaccine can be formulated for storage at ambient temperatures.

Plans for technology transfer of RNA vaccine candidate to developing country vaccine manufacturer: The advantage of proposed iRNA based technology is the low vaccine cost, rapid production, affordable transportation and storage, as well as user-friendly manufacturing.

The iRNA-based vaccine can induce a broader immune response than the standard mRNA or DNA vaccines due to the immunostimulatory effects of iRNA and live-attenuated virus. In addition to improved safety and immunogenicity, rearranged iRNA has an advantage in manufacturing, storage, genetic stability, and transportation with no need for a cold chain.

### Example 15

### Rescue of V5040 Live-Attenuated CHIKV Vaccine from pMG5040 iDNA Plasmid in Vitro

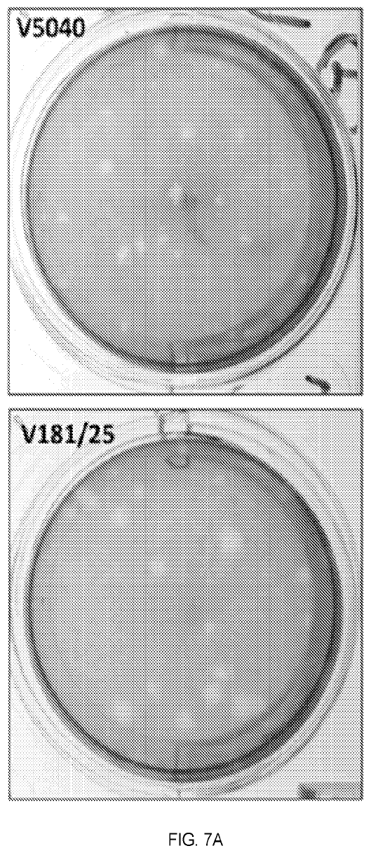

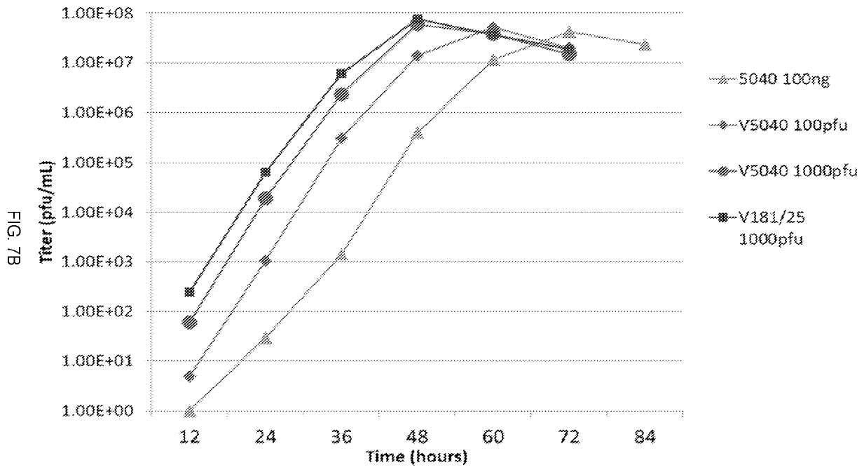

The iDNA plasmid containing the full-length iDNA was isolated from E. coli (FIG. 4B) and evaluated in cultured cells. The genomic RNA transcription from iDNA is expected to occur in the nucleus and the genomic RNA is transported to the cytoplasm where translation and virus synthesis takes place. To evaluate if V5040 CHIKV with rearranged structural genes can be launched in vitro from the plasmid, pMG5040 was transfected into Vero cells (ATCC CCL-81.5). Vero cells are often used in the vaccine manufacturing. Expression of replication competent (infectious) virus was confirmed in the medium from transfected cells by plaque assay. As seen in FIGS. 7A and 7B. incubation of cells with pMG5040-transfected medium (see Materials and Methods) generated plaques under agarose overlay at 12-24 h. p. i. (FIGS. 7A and 7B). Similar to CHIKV 181/25-generated plaques, V5040 plaques were heterogeneous and appeared to be smaller in diameter. The V5040 virus harvested from the supernatant at 48 h post-transfection had an infectious titer 108 PFU/mL indicating successful rescue of replication-competent virus from pMG5040 iDNA plasmid and efficient replication of V5040 in Vero cells. Expression of CHIKV-specific antigens in V5040-infected cells was confirmed by IFA using CHIKV-specific antiserum VR-1241AF as described elsewhere.

In the next experiment, we compared replication of V5040 virus in Vero cells infected either with the infectious V5040 (100 PFU or 1000 PFU representing MOI of 10⁻⁵ and 10⁻⁴, respectively), or transfected with pMG5040 plasmid. As a control, we used experimental CHIKV 181/25 vaccine.

Samples of the culture medium from transfected and infected cells were collected every 12 h and the viral titers were quantitated by plaque assay. As shown in FIG. 7B, infection at MOI of 10⁻⁵ resulted in effective virus replication comparable with CHIKV 181/25 kinetics and reached 108 PFU/ml at 48 h post infection. Infection at lower MOI 10⁻⁴ had similar kinetics peaking at 60 h. p. i. In pMG5040-transfected cells, the majority of cells expressed CHIKV antigens at 48 h post-transfection and infectious virus in culture medium approached 108 PFU/ml at 72 h. Without being bound to a particular theory, we think observed delay of the peak titer in pMG5040 transfected cells as compared to V5040 infected cells can be explained by the timing associated with cell recovery after pMG5040 transfection, DNA penetrating the nuclei of transfected cells, transcription of RNA and transport into cytoplasm to start replication of V5040. Therefore, replication of V5040 virus from plasmid is expected to be delayed causing peak titer at a later point as compared to the control CHIKV 181/25 virus. Similar observation was made with related rearranged alphavirus.

Based on our experiments, we observed that infection 100-1000 PFU of V5040 virus, or transfection with 100 ng of pMG5040 iDNA efficiently initiated replication of live attenuated CHIKV in vitro.

### Example 16

### Immunogenicity of V5040 Vaccine in Mice

To further secure attenuation and safety of the experimental V5040 vaccine, attenuating mutations E2 Thr12Ile and Gly82Arg derived from the CHIKV 181/25 vaccine, which is currently under clinical evaluation, were introduced in the GP region of the pMG5040 and V5040.

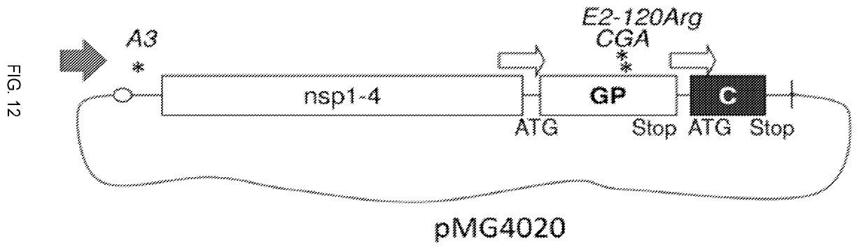

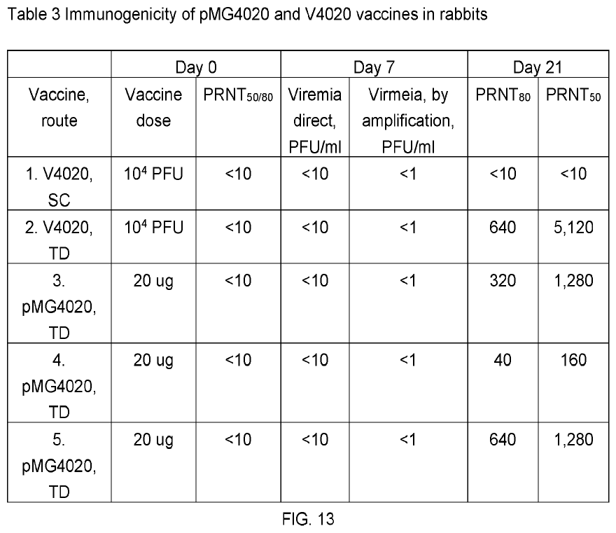

In order to test safety and immunogenicity of rearranged V5040 CHIKV vaccine, we used BALB/c and C57BL/6 mice. In the first experiment, BALB/c mice were vaccinated with a single s.c. dose of 10⁴ PFU or 10⁵ of V5040 virus prepared from the growth medium of pMG5040-transfected Vero cells. Similarly, 181/25 virus was administered as control. After injections, all mice remained healthy with no detectable adverse effects such as changes in weight or behavior. Serum samples were collected as described in Materials and Methods. Viremia was not detectable in V5040-vaccinated mice by direct plaque assay (detection limit 25 PFU/ml). However, in 40% of the vaccinated mice, low viremia was detected by co-cultivation assay (FIG. 11, Table 2). At day 28, mice seroconverted as determined by IFA and western blot (FIGS. 8A and 8B; and FIG. 11, Table 2). Similarly, BALB/c mice vaccinated with a single s.c. dose of 10⁵ PFU of V5040 virus did not show any safety concerns and developed antibody response (FIG. 11, Table 2).

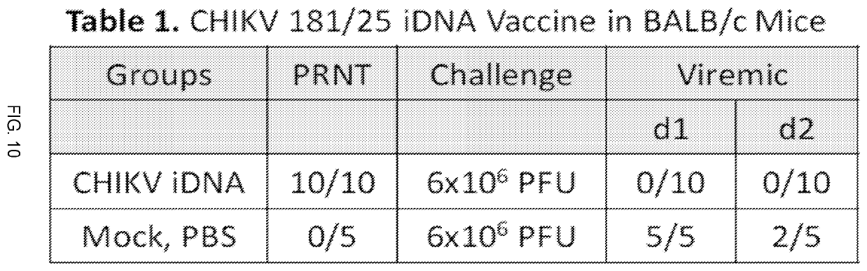

Next, we evaluated neutralization activity of V5040 vaccine with antisera from vaccinated C57BL/6 mice using a PRNT50 assay. As control, we used 181/25 vaccine. Both vaccines demonstrated measurable neutralization activity starting from 7 days post vaccination after a single dose of vaccine (FIG. 9). Compared to 181/25 CHIKV vaccine, iDNA-based V5040 vaccine induced stronger and more durable PRNT50 neutralizing titers. While approximately 50% (4-5/8 mice, per group) of 181/25 vaccinated mice showed a measurable neutralization activity (>1:32), nearly all of V5040 vaccinated mice demonstrated PRNT50 >1:128 after 14 days post vaccination. Interestingly enough, V5040 showed a better longevity of neutralization activity, showing a significantly higher PRNT50 titer compared to CHIKV 181/25 at 42 days post vaccination (p<0.0007. Two-way ANOVA, mixed effect model).

### Example 17

### Administering the iDNA VEEV Vaccine by Microneedle Injection

Nucleic acid vaccines can be delivered using the hMTS platform, which is designed for the delivery of non-viscous liquid formulations. The cartridges for hMTS devices were aseptically filled with 0.5 ml of vaccines (either pMG4020 iDNA or V4020 virus) according to the manufacturer's instructions. Three hMTS devices were filled with pMG4020, while one hMTS device was filled with control V4020 live virus vaccine. The filled cartridges were crimped to properly seal the cartridge. Immediately before administering the vaccine to animals, the vaccine-filled cartridges were inserted into the injector to align with the spring-powered vaccine delivery plunger mechanism. The hMTS injector contained an array of twelve preinstalled hollow microneedles to administer the vaccine to the injection site.

Animal research involving experimental rabbits was done according to the approved institutional protocol (Noble Life Sciences No. 493). New Zealand white rabbits were purchased from Charles River. Animals were divided into two groups, three per group. Rabbits (2.5-3 kg, adult, female) were anesthetized prior to vaccinations. One group of three animals was vaccinated using pMG4020 plasmid encoding the V4020 virus. Another group was vaccinated using the V4020 virus. Either vaccine (pMG4020 iDNA plasmid or V4020 virus) was administered using hMTS microneedle devices according to the manufacturer's instructions. To minimize the number of animals due to ethical and animal welfare reasons, one control rabbit was injected with V4020 vaccine virus using the hMTS device, and one control rabbit was injected with V4020 vaccine virus using a standard syringe needle via subcutaneous (SC) route of administration.

The vaccine was administered on day 0. Animals were vaccinated with 0.5 ml of pMG4020 iDNA (20 mg) or V4020 virus vaccine (10⁴ PFU) using hMTS microneedle devices or with 0.5 ml (10⁴ PFU) of V4020 vaccine SC using a standard syringe in the right leg at a titer of 2×10⁴ PFU/ml. The human dose of the TC83 vaccine is 10⁵ PFU; therefore, 10⁴ PFU dose was chosen for rabbits considering the difference in body weight.

The animals were placed under general anesthesia using a combination of ketamine (50 mg/kg) and xylazine (10 mg/kg), and the site of injection was cleanly shaved using clippers followed by a razor prior to application of the device and injection. The injection site skin was stretched and secured onto a solid support bar. The device was attached to the skin via a self-adhesive surface, and the injection button on the device was depressed to deliver the pre-loaded vaccine cargo from the cartridge through the microneedles. As a control, one animal was dosed SC with 0.5 ml of the attenuated VEE vaccine strain V4020 at the same amount and concentration as those in the hMTS microneedle array device.

After vaccinations, animals were observed daily. Blood samples were taken on days 0, 7, and 21. On days 0 (vaccination), 7, and 21, rabbits were bled via the auricular artery for assessment of viremia (day 7) and the humoral response (neutralizing antibodies and IFA) to the vaccines. In a previous study, viremia was detectable up to 8 days in cotton rats after SC inoculation with 3 log10 PFU of VEEV.

Results show that the iDNA encoding live-attenuated VEEV vaccine delivered by iDNA using this approach is immunogenic in vivo.

### Example 18

### Administering the iDNA CHIKV Vaccine by Microneedle Injection

iDNA of full-length attenuated CHIKV can be delivered in vivo similar to the method as described in Example 17. Results show that the iDNA encoding live-attenuated CHIKV vaccine delivered by iDNA using this approach is immunogenic in vivo.

### Example 19

### Immunogenicity Following in Direct Administering of the iRNA In Vivo

BALB/c mice (4-8 week-old, 5 mice per group, Noble Life Sciences, Woodbine, MD) are anesthetized with isoflurane prior to vaccinations. Each experiment is done at least two times to ensure reproducibility. Mice are vaccinated subcutaneously (s.c.) with 0.1 or 10 ug iRNA of full length live-attenuated CHIKV formulated with (i) saline, (ii) liposome formulation, or intradermally (i.d.) with CHIKV iRNA (same dosage) using microneedles. As control CHIKV 181/25 vaccine virus is used s.c. via standard needle. As another control, iDNA encoding iRNA is used (same dosage). Vaccinations are done in the dorsal area. Alternatively, vaccination with iRNA is carried out intramuscularly (i.m.) with the same dose, followed by in vivo electroporation at the site of injection. After vaccinations, animals are observed daily for clinical signs of infection and body weights. Blood samples are collected from the retro-orbital sinus 2-5 days after vaccination to detect CHIKV in the blood (viremia), and on day 28 to detect immune response. Mice are then transferred BSL3 facility and challenged with virulent CHIKV at a dose of 1×10⁴ PFU in 20 ul by the s.c. route, in order to detect if protection is induced by vaccination with iRNA or control virus. After challenge, animals are observed daily for clinical signs of infection and body weight. Morbidity (body weight, behavior changes) is determined. Sera are taken on day 3 post challenge, and representative mice from each challenge group are sacrificed on day 3 to examine virus titers in the tissues. Results show that iRNA induces immune response when given using various routes of administration and dosage.

### REFERENCES

The following publications, references, patents and patent applications are hereby incorporated by reference in their entireties.
Graham, B. S.; Repik, P. M.; Yactayo, S., Chikungunya in the Americas: Recommendations and Conclusions. J Infect Dis 2016, 214 (suppl 5), S510-S513.
Edelman, R.; Tacket, C. O.; Wasserman, S. S.; Bodison, S. A.; Perry, J. G.; Mangiafico, J. A., Phase II safety and immunogenicity study of live chikungunya virus vaccine TSI-GSD-218. Am J Trop Med Hyg 2000, 62 (6), 681-5.
Tretyakova, I.; Hear, J.; Wang, E.; Weaver, S.; Pushko, P., DNA vaccine initiates replication of live attenuated chikungunya virus in vitro and elicits protective immune response in mice. J Infect Dis 2014, 209 (12), 1882-90.
Hidajat, R.; Nickols, B.; Forrester, N.; Tretyakova, I.; Weaver, S.; Pushko, P., Next generation sequencing of DNA-launched Chikungunya vaccine virus. Virology 2016, 490, 83-90.
Pushko, P., Lukashevich, I. IDNA vaccines and methods for using the same U.S. Pat. No. 8,691,563B2, 2009.
Pushko, P., Tretyakova, I., Lukashevich, I. Infectious DNA vaccines against chikungunya virus. U.S. Pat. No. 9,101,572B2.
Schwameis, M.; Buchtele, N.; Wadowski, P. P.; Schoergenhofer, C.; Jilma, B., Chikungunya vaccines in development. Human Vaccines & Immunotherapeutics 2016, 12 (3), 716-731.
Plante, K. S.; Rossi, S. L.; Bergren, N. A.; Seymour, R. L.; Weaver, S. C., Extended Preclinical Safety, Efficacy and Stability Testing of a Live-attenuated Chikungunya Vaccine Candidate. PLOS Neglected Tropical Diseases 2015, 9 (9), e0004007.
Bull, J. J., Evolutionary reversion of live viral vaccines: Can genetic engineering subdue it? Virus Evol 2015, 1 (1).
Tretyakova, I.; Lukashevich, I. S.; Glass, P.; Wang, E.; Weaver, S.; Pushko, P., Novel vaccine against Venezuelan equine encephalitis combines advantages of DNA immunization and a live attenuated vaccine. Vaccine 2013, 31 (7), 1019-25.
Pushko, P.; Lukashevich, I. S.; Weaver, S. C.; Tretyakova, I., DNA-launched live-attenuated vaccines for biodefense applications. Expert Rev Vaccines 2016, 15 (9), 1223-34.
Ziegler, S. A.; Lu, L.; da Rosa, A. P.; Xiao, S. Y.; Tesh, R. B., An animal model for studying the pathogenesis of chikungunya virus infection. Am J Trop Med Hyg 2008, 79 (1), 133-9.
Morrison, T. E.; Oko, L.; Montgomery, S. A.; Whitmore, A. C.; Lotstein, A. R.; Gunn, B. M.; Elmore, S. A.; Heise, M. T., A mouse model of chikungunya virus-induced musculoskeletal inflammatory disease: evidence of arthritis, tenosynovitis, myositis, and persistence. Am J Pathol 2011, 178 (1), 32-40.
Haese, N. N.; Broeckel, R. M.; Hawman, D. W.; Heise, M. T.; Morrison, T. E.; Streblow, D. N., Animal Models of Chikungunya Virus Infection and Disease. J Infect Dis 2016, 214 (suppl 5), S482-S487.
Tretyakova, I.; Tibbens, A.; Jokinen, J. D.; Johnson, D. M.; Lukashevich, I. S.; Pushko, P., Novel DNA-launched Venezuelan equine encephalitis virus vaccine with rearranged genome. Vaccine 2019, 37 (25), 3317-3325.
Glass, P. J., Lybarger, E. A., Teehee, M. L. and Parker, M. D., Chikungunya virus strain TSI-GSD-218-VR1. GenBank Accession No. EF452494. 2007.
Watanabe, Y.; Hamaguchi-Tsuru, E.; Morimoto, N.; Nishio, Y.; Yagyu, K.; Konishi, Y.; Tominaga, M.; Miyazaki, J.; Furuya, M.; Tominaga, A., IL-5-Induced Eosinophils Suppress the Growth of Leishmania amazonensis In Vivo and Kill Promastigotes In Vitro in Response to Either IL-4 or IFN-gamma. DNA Cell Biol 2004, 23 (7), 412-8.
Roy, C. J.; Adams, A. P.; Wang, E.; Plante, K.; Gorchakov, R.; Seymour, R. L.; Vinet-Oliphant, H.; Weaver, S. C., Chikungunya vaccine candidate is highly attenuated and protects nonhuman primates against telemetrically monitored disease following a single dose. J Infect Dis 2014, 209 (12), 1891-9.

Jeffs, L. B.; Palmer, L. R.; Ambegia, E. G.; Giesbrecht, C.; Ewanick, S.; MacLachlan, I., A scalable, extrusion-free method for efficient liposomal encapsulation of plasmid DNA. Pharm Res 2005, 22 (3), 362-72.

Muramatsu, H.; Lam, K.; Bajusz, C.; Laczko, D.; Kariko, K.; Schreiner, P.; Martin, A.; Lutwyche, P.; Heyes, J.; Pardi, N., Lyophilization provides long-term stability for a lipid nanoparticle-formulated, nucleoside-modified mRNA vaccine. Mol Ther 2022.

Shen, N. L.; Hartikka, J.; Horn, N. A.; Manthorpe, M.; Marquet, M., Development and characterization of lyophilized DNA vaccine formulations. Methods Mol Med 2000, 29, 23-34.
Akahata, W., et al., A virus-like particle vaccine for epidemic Chikungunya virus protects nonhuman primates against infection. Nat Med, 2010. 16 (3): p. 334-8.
Arankalle, V. A., et al., Genetic divergence of Chikungunya viruses in India (1963-2006) with special reference to the 2005-2006 explosive epidemic. J Gen Virol, 2007. 88(Pt 7): p. 1967-76.
BPROM. Available from: http://linux1.softberry.com/berry.phtml?topic=bprom&group=programs&subgroup=gfind b.
Brehin, A. C., et al., The large form of human 2′,5′-Oligoadenylate Synthetase (OAS3) exerts antiviral effect against Chikungunya virus. Virology, 2009. 384(1): p. 216-22.
Broeckel, R., et al., Nonhuman Primate Models of Chikungunya Virus Infection and Disease (CHIKV NHP Model). Pathogens, 2015. 4(3): p. 662-81.
Burt, F. J., et al., Chikungunya virus: an update on the biology and pathogenesis of this emerging pathogen. Lancet Infect Dis, 2017. 17(4): p. e107-e117.
Couderc, T., et al., A mouse model for Chikungunya: young age and inefficient type-I interferon signaling are risk factors for severe disease. PLOS Pathog, 2008. 4(2): p. e29.
Dhiman, R. C., et al., Climate change and threat of vector-borne diseases in India: are we prepared? Parasitol Res, 2010. 106(4): p. 763-73.
Enserink, M., Entomology. A mosquito goes global. Science, 2008. 320(5878): p. 864-6.
Enserink, M., INFECTIOUS DISEASES. An obscure mosquito-borne disease goes global. Science, 2015. 350(6264): p. 1012-3.
Epstein, P. R., Chikungunya Fever resurgence and global warming. Am J Trop Med Hyg, 2007. 76(3): p. 403-4.
Flanagan, E. B., T. R. Schoeb, and G. W. Wertz, Vesicular stomatitis viruses with rearranged genomes have altered invasiveness and neuropathogenesis in mice. J Virol, 2003. 77(10): p. 5740-8.
Jain, M., S. Rai, and A. Chakravarti, Chikungunya: a review. Trop Doct, 2008. 38(2): p. 70-2.
Johnson, D. M., et al., Advanced Safety and Genetic Stability in Mice of a Novel DNA-Launched Venezuelan Equine Encephalitis Virus Vaccine with Rearranged Structural Genes. Vaccines (Basel), 2020. 8(1).
Josseran, L., et al., Chikungunya disease outbreak, Reunion Island. Emerg Infect Dis, 2006. 12(12): p. 1994-5.
Lanciotti, R. S., et al., Chikungunya virus in US travelers returning from India, 2006. Emerg Infect Dis, 2007. 13(5): p. 764-7.
Muthumani, K., et al., Immunogenicity of novel consensus-based DNA vaccines against Chikungunya virus. Vaccine, 2008. 26(40): p. 5128-34.
Open Reading Frame Finder. Available from: https://www.ncbi.nlm.nih.gov/orffinder/.
Petersen, L. R., S. L. Stramer, and A. M. Powers, Chikungunya virus: possible impact on transfusion medicine. Transfus Med Rev, 2010. 24(1): p. 15-21.
Pistone, T., et al., An imported case of Chikungunya fever from Madagascar: use of the sentinel traveller for detecting emerging arboviral infections in tropical and European countries. Travel Med Infect Dis, 2009. 7(1): p. 52-4.
Plotkin, S., et al., The complexity and cost of vaccine manufacturing—An overview. Vaccine, 2017. 35(33): p. 4064-4071.
Pumpens, P., Pushko, P., Virus-Like Particles: A Comprehensive Guide. 2022.
Pushko, P., Parker, M. D., Smith, J. F., Crise, B. J., Live attenuated Venezuelan Equine encephalitis vaccine. U.S. Pat. No. 6,296,854, USPTO, Editor. 2001.
Queyriaux, B., et al., Clinical burden of chikungunya virus infection. Lancet Infect Dis, 2008. 8(1): p. 2-3.
Ramsauer, K., et al., Immunogenicity, safety, and tolerability of a recombinant measles-virusbased chikungunya vaccine: a randomised, double-blind, placebo-controlled, active-comparator, first-in-man trial. Lancet Infect Dis, 2015. 15(5): p. 519-27.
Rouxel, R. N., et al., Attenuated Infectious Hematopoietic Necrosis Virus with Rearranged Gene Order as Potential Vaccine. J Virol, 2016. 90(23): p. 10857-10866.
Sam, I. C. and S. AbuBakar, Chikungunya virus infection. Med J Malaysia, 2006. 61(2): p. 264-9.
Schwartz, O. and M. L. Albert, Biology and pathogenesis of chikungunya virus. Nat Rev Microbiol, 2010. 8 (7): p. 491-500.
Splice Site Prediction by Neural Network. Available from: https://www.fruitfly.org/seq_tools/splice.html.
Thiboutot, M. M., et al., Chikungunya: a potentially emerging epidemic? PLOS Negl Trop Dis, 2010. 4(4): p. e623.
Tiwari, M., et al., Assessment of immunogenic potential of Vero adapted formalin inactivated vaccine derived from novel ECSA genotype of Chikungunya virus. Vaccine, 2009. 27(18): p. 2513-22.
Tretyakova, I., et al., Plasmid DNA initiates replication of yellow fever vaccine in vitro and elicits virus-specific immune response in mice. Virology, 2014. 468-470: p. 28-35.
Tretyakova, I., et al., Venezuelan equine encephalitis vaccine with rearranged genome resists reversion and protects non-human primates from viremia after aerosol challenge. Vaccine, 2020. 38(17): p. 3378-3386.
Tsetsarkin, K. A., et al., A single mutation in chikungunya virus affects vector specificity and epidemic potential. PLOS Pathog, 2007. 3(12): p. e201.
Vazeille, M., et al., Two Chikungunya isolates from the outbreak of La Reunion (Indian Ocean) exhibit different patterns of infection in the mosquito, Aedes albopictus. PLOS One, 2007. 2(11): p. e1168.
Wang, E., et al., Chimeric alphavirus vaccine candidates for chikungunya. Vaccine, 2008. 26(39): p. 5030-9.
Weaver, S. C. and W. K. Reisen, Present and future arboviral threats. Antiviral Res, 2010. 85(2): p. 328-45.
Wressnigg, N., et al., Single-shot live-attenuated chikungunya vaccine in healthy adults: a phase 1, randomised controlled trial. Lancet Infect Dis, 2020. 20(10): p. 1193-1203. 22.
Zeller, H., W. Van Bortel, and B. Sudre, Chikungunya: Its History in Africa and Asia and Its Spread to New Regions in 2013-2014. J Infect Dis, 2016. 214(suppl 5): p. S436-S440.
Szurgot, I., et al., Infectious RNA vaccine protects mice against chikungunya virus infection. Sci Rep, 2020, 10:21076.
Tretyakova, I, et al., Live-Attenuated VEEV Vaccine Delivered by iDNA Using Microneedles Is Immunogenic in Rabbits. Front Trop Dis, 2022, 3:813671.

1. An infectious RNA (iRNA) molecule comprising an RNA encoding an attenuated virus;
optionally the attenuated virus is an emerging virus;
optionally the attenuated virus is a positive-strand RNA virus;
optionally the positive-strand RNA virus is selected from the group consisting of one or more Ribovirus, Orthornavirus, Kitrinovirus, Lenarvirus, Pisuvirus, Pisonivirus, Stelpavirus, Togavirus, and combinations thereof;
optionally positive-strand RNA virus is selected from the group consisting of:
one or more Alphavirus such as western equine encephalitis virus (WEEV), Venezuelan equine encephalitis virus (VEEV), eastern equine encephalitis virus (EEEV), Sindbis virus (SINV), Semliki Forest virus (SFV), and Chikungunya virus (CHIKV),
one or more Flavivirus, such as Hepacivirus C, yellow fever virus (YFV), West Nile virus, Dengue virus, and Japanese encephalitis virus (JEV),
one or more Arenavirus, such as Lassa virus, Guanarito virus, Junin virus, Lujo virus, Machupo virus, Sabia virus, and Whitewater Arroyo virus,
one or more Coronavirus, such as Middle East respiratory syndrome (MERS) virus, Severe Acute Respiratory Syndrome (SARS) virus, and SARS-Coronavirus 2 (SARS-COV-2),
one or more Picornavirus, such as rhinovirus and common cold virus, one or more retrovirus, such as simian immunodeficiency virus (SIV), human immunodeficient virus (HIV), simian type D retrovirus (SRV), and simian T-lymphotropic virus (STLV),
and combinations thereof;
optionally the positive-strand RNA virus is selected from the group consisting of one or more CHIKV, VEEV, YFV, JEV, and combinations thereof;
optionally the positive-strand RNA virus is selected from the group consisting of one or more CHIKV, VEEV, and combinations thereof;
optionally the positive-strand RNA virus is a CHIKV;
optionally the positive-strand RNA virus is VEEV;
optionally the positive-strand RNA virus is selected from the group consisting of a 181/25 CHIKV, a V5040 CHIKV, a TC85 VEEV, V4020 VEEV, and combinations thereof; and
optionally the attenuated virus is encoded by the sequence of any one of SEQ. 1 to 4.

2. The iRNA molecule of claim 1,
wherein the iRNA molecule encodes all of the gene products of the attenuated virus;
optionally the iRNA molecule encodes a full-length RNA from the attenuated virus;
optionally the iRNA molecule is a recombinant RNA molecule;
optionally the iRNA molecule encodes one or more attenuating mutations;
optionally the one or more attenuating mutations is a point mutation, a rearrangement, and/or combinations thereof;
optionally the one or more attenuating mutation excludes a non-rearrangement deletion of more than 180 bp, optionally no more than about 175 bp, optionally no more than about 170 bp, optionally no more than about 170 bp, optionally no more than about 165 bp, optionally no more than about 160 bp, optionally no more than about 155 bp, optionally no more than about 150 bp, optionally no more than about 125 bp, more optionally no more than about 100 bp, optionally no more than about 75 bp, optionally no more than about 55 bp, optionally no more than about 50 bp, optionally no more than about 25 bp, optionally no more than about 20 bp, optionally no more than about 15 bp, optionally no more than about 10 bp, optionally no more than about 5 bp, optionally no more than about 0 bp;
optionally the one or more attenuating mutation excludes a non-rearrangement deletion of nucleotides encoding no more than 58 amino acids, optionally no more than about 55 amino acids, optionally no more than about 50 amino acids, optionally no more than about 45 amino acids, optionally no more than about 40 amino acids, more optionally no more than about 35 amino acids, optionally no more than about 30 amino acids, optionally no more than about 25 amino acids, optionally no more than about 20 amino acids, optionally no more than about 15 amino acids, optionally no more than about 10 amino acids, optionally no more than about 5 amino acids, optionally no more than about 0 amino acids;
optionally the iRNA molecule encodes a full-length nsP3 gene when the attenuated virus is a CHIKV;
optionally the iRNA molecule encodes a full-length 6K gene when the attenuated virus is a CHIKV;
optionally the iRNA molecule encodes two attenuating point mutations and/or a rearrangement,
optionally the rearrangement is between the capsid gene and the glycoprotein gene;
optionally the one or more of the point mutation is at the glycoprotein gene; optionally the one or more point mutation is at the envelop gene;
and optionally the one or more point mutation is at the E2 gene of the CHIKV, and optionally one or both of a Thr12Ile and Gly82Arg mutation of the E2 gene of the CHIKV;
optionally one or more mutations selected from in SEQ. 1 and/or SEQ 3;
optionally the mutation is substantially stable, has substantially reduced rates of reversion, is substantially resistant to reversions, and/or combinations thereof;
optionally the mutation is highly stable, has highly reduced rates of reversion, is highly resistant to reversions, and/or combinations thereof;
optionally the mutation does not reverse to a more virulent virus; and
optionally the mutation has no detectable reversions;
optionally the mutation is substantially stable, or highly stable, or has no detectable reversions following a direct or an indirect administration of the iRNA into a subject in need thereof;
optionally the mutation is substantially stable, or highly stable, or has no detectable reversions during the entire course of treatment following a direct or an indirect administration of the iRNA into a subject in need thereof;
optionally the mutation does not impair the replication of attenuated virus in a mammalian cell, optionally a primate cell, optionally a human cell;
optionally the mutation is stable;
optionally the iRNA molecule comprises one or more subgenomic promoters,
wherein the one or more, optionally two or more, subgenomic promoters are operably linked to the iRNA molecule,
optionally the one or more subgenomic promoters is an RNA polymerase promoter, optionally an eukaryotic promoter or a bacteriophage promoter;
optionally the one or more subgenomic promoters is one or more 26S promoters;
optionally the one or more subgenomic promoters is two 26S promoters;
optionally each subgenomic promoter is operably linked to a gene encoded by the iRNA molecule;
optionally the first subgenomic promoter is operatively linked to a capsid gene and the second subgenomic promoter is operatively linked to a glycoprotein gene; and
optionally the glycoprotein gene is E3-E2-6K-E1; and
more optionally the iRNA molecule has a sequence of SEQ. 1 and/or SEQ. 3.

3. An infectious DNA (iDNA) molecule encoding any one of the iRNA molecule in claim 1,
wherein the iDNA is operatively linked to a DNA-dependent RNA polymerases production promoter,
optionally the production promoter is suitable for manufacturing the encoded iRNA molecule; optionally for the iRNA molecule manufactured is pharmaceutically acceptable;
optionally the production promoter is a bacteriophage promoter;
optionally the production promoter is a prokaryotic promoter;
optionally the production promoter is selected from the group consisting of T3, T7 and 26S;
optionally the production promoter is distinct from the subgenomic promoter;
optionally the iDNA molecule is carried in a DNA plasmid;
optionally the iDNA molecule is carried in a pT7 plasmid;
optionally the iDNA molecule is carried in a pMG plasmid;
optionally the DNA plasmid is a pMG4020 plasmid and/or a pMG5040 plasmid;
optionally the iDNA has the a sequence of SEQ. 2 and/or SEQ. 4;
wherein the iDNA further comprise an eukaryotic promoter, optionally the promoter is optimized;
optionally a mammalian promoter,
optionally a CMV promoter, and
optionally an optimized hybrid human CMV (hCMV) promoter;
optionally the iDNA molecule encodes a full-length RNA from the attenuated virus;
optionally the eukaryotic promoter and/or production promoter is located upstream of the iRNA encoded by the iDNA molecule;
optionally the iDNA molecule is stable, optionally thermal and/or genetically stable;
optionally the iDNA molecule is characterized by being sterile, and/or having about 95% of the iDNA molecule being supercoiled, and/or having an A260/A280 ratio of selected from the group consisting of from about 1.6 to about 2.2, from about 1.7 to about 2.1, from about 1.8 to about 2.0, and about 1.9;
optionally the iDNA molecule is capable of inducing an immune response;
optionally the immune response is an innate immune response, an adaptive immune response, and/or combinations thereof;
optionally the immune response is a long-lasting immunity;
optionally a broadly cross-neutralizing immune response; and
optionally the iDNA molecule is capable of inducing the immune response in a single dose;
optionally the iDNA molecule is pharmaceutically acceptable;
optionally the iDNA molecule is manufactured in vitro or in vivo or by direct synthesis; optionally, the iDNA molecule is manufactured in vitro;
optionally the iDNA is manufactured under GMP conditions;
optionally the iDNA is manufactured for vaccine use;
optionally the iDNA is formulated in a composition further comprising a pharmaceutically acceptable non-toxic component;
optionally, the component is a saline and/or a buffer; and optionally the saline is a phosphate buffered solution;
optionally the composition is pharmaceutically acceptable;
optionally the composition and/or iDNA molecule is suitable for direct administration in a subject in need thereof;
optionally the composition and/or the iDNA molecule is capable of inducing an immune response in a subject in need thereof through an iRNA,
optionally the immune response is an innate immune response, or an adaptive immune response, and/or combinations thereof;
optionally the immune response is a long-lasting immunity;
optionally the immune response is a broadly cross-neutralizing immune response; and
optionally the composition and/or the iDNA molecule is capable of inducing the immune response in one or more doses, optionally a single dose;
optionally the composition and/or the iDNA molecule is suitable for a use as a vaccine;
optionally the iDNA molecule is suitable for a use as a vaccine;
optionally the virus is suitable for reducing and/or substantially minimizing one or more symptoms in a subject in need thereof,
wherein the subject is optionally a mammal, more optionally a human; and
optionally the iDNA molecule is used to reduce or substantially minimize a viral infection and/or diminish the symptoms of a viral infection; and
optionally the symptoms are selected from the group consisting of a fever, headache, rash, nausea, myalgia, arthralgia, respiratory failure, cardiovascular disease, hepatitis, cutaneous effects, central nervous system abnormalities, and combinations thereof.

4. A composition comprising the iRNA molecule of claim 1, further comprising a pharmaceutically acceptable non-toxic component;
optionally, the component is a saline and/or a buffer; and optionally the saline is a phosphate buffered solution;
optionally the iRNA molecule is pharmaceutically acceptable;
optionally the iRNA molecule is manufactured in vitro or in vivo or by direct synthesis; more optionally, iRNA molecule is manufactured in vitro;
optionally the iRNA is manufactured under GMP conditions;
optionally the iRNA is manufactured for vaccine use;
optionally the iRNA molecule is manufactured using the iDNA of claim 3;
optionally the composition is pharmaceutically acceptable;
optionally the composition and/or iRNA molecule is suitable for direct administration in a subject in need thereof;
optionally the composition and/or the iRNA molecule is capable of inducing an immune response following direct administration in a subject in need thereof,
optionally the immune response is an innate immune response, or an adaptive immune response, and/or combinations thereof;
optionally the immune response is a long-lasting immunity;
optionally the immune response is a broadly cross-neutralizing immune response; and
optionally the composition and/or the iRNA molecule is capable of inducing the immune response in one or more doses, optionally a single dose;
optionally the composition and/or the iRNA molecule is suitable for a use as a vaccine;
optionally the composition and/or the iRNA molecule is suitable for reducing and/or substantially minimizing one or more symptoms in a subject in need thereof,
wherein the subject is optionally a mammal, more optionally a human; and
optionally the iRNA molecule is used to reduce or substantially minimize a viral infection and/or diminish the symptoms of a viral infection; and
optionally the symptoms is selected from the group consisting of a fever, headache, rash, nausea, myalgia, arthralgia, respiratory failure, cardiovascular disease, hepatitis, cutaneous effects, central nervous system abnormalities, and combinations thereof.

5. A vehicle comprising an infectious genetic material;
optionally the vehicle encapsulates the infectious genetic material;
optionally the infectious genetic material is the iRNA molecule of claim 1, the iDNA molecule of claim 3, the composition of claim 4, and/or combinations thereof;
optionally the vehicle is a nanoparticle, including a liposome and a lipid nanoparticle, and including a cationic lipid 1,2-dioleoyl-3-timethylammonium-propane (DOTAP) lipid-based lipid nanoparticle;
optionally the lipid nanoparticle comprising the infectious genetic material is formed by mixing the iRNA molecule with a PEGylated and cationic lipid;
optionally the lipid nanoparticle encapsulate the initial infectious genetic material at an amount of more than about 60%, optionally more than about 70% optionally more than about 80%, optionally more than about 90%, optionally more than about 91%, optionally more than about 92%, optionally more than about 93%, optionally more than about 94%, optionally more than about 95%, optionally more than about 96%, optionally more than about 97%, optionally more than about 98%, optionally more than about 99%, optionally more than about 99.9%;
optionally the lipid nanoparticle has an average diameter of about 5 nm to about 200 nm; optionally about 10 nm to about 150 nm; optionally about 20 nm to about 100 nm; optionally about 30 nm to about 90 nm;
optionally about 40 nm to about 85 nm; preferablyoptionally about 50 nm to about 80 nm; preferablyoptionally about 60 nm to about 75 nm; preferablyoptionally about 75 nm;
optionally the lipid nanoparticle are unilamellar vesicles;
optionally the lipid nanoparticle comprise infectious genetic material per lipid nanoparticle at a range selected from the group consisting of about 1 to about 100, about 1 to about 90, about 1 to about 80, about 1 to about 70, about 1 to about 60, about 1 to about 50, about 1 to about 40, about 1 to about 30, about 1 to about 20, about 1 to about 10, about 1 to about 5, about 1 to about 4, about 1 to about 3, about 1 to about 2, about 1 infectious genetic material per lipid nanoparticle;
optionally the vehicle is capable of delivering the infectious genetic material to a subject in need thereof;
optionally the vehicle is capable of delivering the infectious genetic material to the cytoplasm of a cell of a subject in need thereof;
optionally the vehicle is capable of inducing an immune response,
optionally an innate immune response, an adaptive immune response, and/or combinations thereof;
optionally a long-lasting immunity; and
optionally a broadly cross-neutralizing immune response;
optionally the vehicle is capable of inducing the immune response in a single dose;
optionally the vehicle is suitable for vaccine use;
optionally the vehicle further comprises a non-toxic excipient thereof and/or an non-toxic adjuvant;
optionally the vehicle comprising the infectious genetic material is stable for storage at ambient temperatures and/or ultra-cold temperatures;
optionally the vehicle comprising the infectious genetic material is stable for storage without the need for ultra-cold temperatures; and
ptionally the vehicle comprising the infectious genetic material is stable for more than 12 weeks following lyophilization, optionally from about 12 weeks to about 52 weeks, optionally from about 12 weeks to about 24 weeks.

6. A virus comprising an infectious genetic material;
wherein the infectious genetic material is the the iRNA molecule of claim 1, the iDNA molecule of claim 3, the composition of claim 4, and/or combinations thereof;
wherein the virus is a recombinant virus;
optionally the virus is homogenously pure, and/or live-attenuated, and/or contain stable and attenuating mutations;
optionally the virus is capable of inducing an immune response;
optionally the immune response is an innate immune response, an adaptive immune response, and/or combinations thereof;
optionally the immune response is a long-lasting immunity; and
optionally the immune response is a broadly cross-neutralizing immune response;
optionally the virus is capable of inducing the immune response in a single dose;
optionally the virus is suitable for use in a vaccine;
optionally the virus is suitable for reducing and/or substantially minimizing one or more symptoms in a subject in need thereof,
wherein the subject is optionally a mammal, more optionally a human; optionally the virus is suitable for administration by injection, including intramuscular injection, and/or, by subcutaneous injection, and/or combinations thereof;
optionally the vehicle is used to reduce or substantially minimize a viral infection and/or prevent or diminish the symptoms of a viral infection; and
optionally the symptoms is selected from the group consisting of a fever, headache, rash, nausea, myalgia, arthralgia, respiratory failure, cardiovascular disease, hepatitis, cutaneous effects, central nervous system abnormalities, and combinations thereof.

7. An vaccine comprising (i) an infectious genetic material and/or a vehicle and (ii) a non-toxic pharmacological excipient thereof,
wherein the infectious genetic material is the the iRNA molecule of claim 1, the iDNA molecule of claim 3, the composition of claim 4, the vehicle is of claims 5, and/or combinations thereof;
optionally the infectious genetic material is homogenously pure;
optionally the infectious genetic material has a low percent of single nucleotide polymorphism;
optionally the pharmacological excipient comprise a preservative and/or a saline; and optionally the saline is a phosphate buffered solution;
optionally the vaccine further comprise an adjuvant;
optionally the vaccine is a multivalent vaccine;
optionally the vaccine comprise an effective amount the attenuated virus;
optionally the effective dose of the vaccine is selected from the group consisting of from about 1 ng to about 500,000 ng, from about 5 ng to about 250,000 ng, from about 6 ng to about 100,000 ng, from about 7 ng to about 50,000 ng, from about 8 ng to about 10,000 ng, from about 9 ng to about 5,000 ng, from about 10 ng to about 1,000 ng, from about 10 ng to about 500 ng, from about 10 ng to about 250 ng, from about 10 ng to about 100 ng, from about 100ng, from about 10 ng to about 50 ng, from about 50 ng to about 500 ng, from about 50 ng to about 1,000 ng, from about 100 ng to about 1,000 ng, from about 100 ng to about 5,000 ng, from about 100 ng to about 10,000 ng, from about 500 ng to about 1,000 ng, from about 500 ng to about 5,000 ng, from about 500 ng to about 10,000 ng, from about 500 ng to about 50,000 ng, from about 500 ng to about 75,000 ng, and from about 500 ng to about 100,000 ng of the infectious genetic material;
optionally the effective dose of the vaccine is selected from the group consisting of from about 1 ng to about 500,000 ng, from about 5 ng to about 250,000 ng, from about 6 ng to about 100,000 ng, from about 7 ng to about 50,000 ng, from about 8 ng to about 10,000 ng, from about 9 ng to about 5,000 ng, from about 10 ng to about 100,000 ng, from about 10 ng to about 75,000 ng, from about 10 ng to about 50,000 ng, from about 10 ng to about 25,000 ng, from about 10 ng to about 10,000 ng, from about 10 ng to about 7,500 ng, from about 10 ng to about 50,000 ng, from about 10 ng to about 25,000 ng, from about 10 ng to about 10,000 ng, from about 10 ng to about 7,500 ng, from about 10 ng to about 5,000 ng, from about 10 ng to about 2,500 ng, from about 10 ng to about 1,000 ng, from about 10 ng to about 500 ng, from about 10 ng to about 250 ng, from about 10 ng to about 120 ng, from about 10 ng to about 100 ng, from about 10 ng to about 50 ng, from about 10 ng to about 75 ng, from about 50 ng to about 500 ng, from about 50 ng to about 1,000 ng, from about 100 ng to about 1,000 ng, from about 100 ng to about 5,000 ng, from about 100 ng to about 10,000 ng, from about 500 ng to about 1,000 ng, from about 500 ng to about 5,000 ng, from about 500 ng to about 10,000 ng, from about 500 ng to about 50,000 ng, from about 500 ng to about 75,000 ng, and from about 500 ng to about 100,000 ng of the iRNA molecule;
optionally the effective dose of the vaccine is selected from the group consisting of from about 1 ng to about 500,000 ng, from about 5 ng to about 250,000 ng, from about 6 ng to about 100,000 ng, from about 7 ng to about 50,000 ng, from about 8 ng to about 10,000 ng, from about 9 ng to about 5,000 ng, from about 10 ng to about 1,200 ng, from about 10 ng to about 1,100 ng, from about 10 ng to about 1,000 ng, from about 10 ng to about 1,200 ng, from about 10 ng to about 500 ng, from about 10 ng to about 250 ng, from about 10 ng to about 120 ng, from about 10 ng to about 100 ng, from about 10 ng to about 50 ng, from about 50 ng to about 500 ng, from about 50 ng to about 1,000 ng, from about 100 ng to about 1,000 ng, from about 100 ng to about 1,100 ng, from about 100 ng to about 1,200, from about 100 ng to about 5,000 ng, from about 100 ng to about 10,000 ng, from about 500 ng to about 1,000 ng, from about 500 ng to about 5,000 ng, from about 500 ng to about 10,000 ng, from about 500 ng to about 50,000 ng, from about 500 ng to about 75,000 ng, and from about 500 ng to about 100,000 ng of the iDNA molecule;
optionally the effective dose of the vaccine is selected from the group consisting of from about 0.5 ng to about 100,000 ng, from about 0.5 ng to about 75,000 ng, from about 0.5 ng to about 50,000 ng, from about 0.5 ng to about 25,000 ng, from about 0.5 ng to about 10,000 ng, from about 0.5 ng to about 7,500 ng, from about 0.5 ng to about 5,000 ng, from about 0.5 ng to about 2,500 ng, from about 0.5 ng to about 1,000 ng, from about 0.5 ng to about 500 ng, from about 0.5 ng to about 250 ng, from about 0.5 ng to about 120 ng, from about 1 ng to about 100,000 ng, from about 1 ng to about 75,000 ng, from about 1 ng to about 50,000 ng, from about 1 ng to about 25,000 ng, from about 1 ng to about 10,000 ng, from about 1 ng to about 7,500 ng, from about 1 ng to about 5,000 ng, from about 1 ng to about 2,500 ng, from about 1 ng to about 1,000 ng, from about 1 ng to about 500 ng, from about 1 ng to about 250 ng, from about 1 ng to about 120 ng, from about 10 ng to about 120 ng, from about 10 ng to about 119 ng, from about 10 ng to about 118 ng, from about 10 ng to about 117 ng, from about 10 ng to about 116 ng, from about 10 ng to about 115 ng, from about 10 ng to about 114 ng, from about 10 ng to about 113 ng, from about 10 ng to about 112 ng, from about 10 ng to about 111 ng, from about 10 ng to about 110 ng, from about 10 ng to about 109 ng, from about 10 ng to about 108 ng, from about 10 ng to about 107 ng, from about 10 ng to about 106 ng, from about 10 ng to about 105 ng, from about 10 ng to about 104 ng, from about 10 ng to about 103 ng, from about 10 ng to about 102 ng, from about 10 ng to about 101 ng, from about 10 ng to about 100 ng, from about 15 ng to about 120 ng, from about 15 ng to about 119 ng, from about 20 ng to about 118 ng, from about 25 ng to about 117 ng, from about 30 ng to about 116 ng, from about 40 ng to about 115 ng, from about 50 ng to about 114 ng, from about 60 ng to about 113 ng, from about 70 ng to about 112 ng, from about 80 ng to about 111 ng, from about 90 ng to about 110 ng of the iDNA molecule, wherein the iDNA molecule encodes a live-attenuated CHIKV;
optionally the effective dose of the vaccine is selected from the group consisting of from about 0.5 ng to about 100,000 ng, from about 0.5 ng to about 75,000 ng, from about 0.5 ng to about 50,000 ng, from about 0.5 ng to about 25,000 ng, from about 0.5 ng to about 10,000 ng, from about 0.5 ng to about 7,500 ng, from about 0.5 ng to about 5,000 ng, from about 0.5 ng to about 2,500 ng, from about 0.5 ng to about 1,000 ng, from about 0.5 ng to about 500 ng, from about 0.5 ng to about 250 ng, from about 0.5 ng to about 120 ng, from about 1 ng to about 100,000 ng, from about 1 ng to about 75,000 ng, from about 1 ng to about 50,000 ng, from about 1 ng to about 25,000 ng, from about 1 ng to about 10,000 ng, from about 1 ng to about 7,500 ng, from about 1 ng to about 5,000 ng, from about 1 ng to about 2,500 ng, from about 1 ng to about 1,000 ng, from about 1 ng to about 500 ng, from about 1 ng to about 250 ng, from about 1 ng to about 120 ng, from about 10 ng to about 120 ng, from about 10 ng to about 119 ng, from about 10 ng to about 118 ng, from about 10 ng to about 117 ng, from about 10 ng to about 116 ng, from about 10 ng to about 115 ng, from about 10 ng to about 114 ng, from about 10 ng to about 113 ng, from about 10 ng to about 112 ng, from about 10 ng to about 111 ng, from about 10 ng to about 110 ng, from about 10 ng to about 109 ng, from about 10 ng to about 108 ng, from about 10 ng to about 107 ng, from about 10 ng to about 106 ng, from about 10 ng to about 105 ng, from about 10 ng to about 104 ng, from about 10 ng to about 103 ng, from about 10 ng to about 102 ng, from about 10 ng to about 101 ng, from about 10 ng to about 100 ng, from about 15 ng to about 120 ng, from about 15 ng to about 119 ng, from about 20 ng to about 118 ng, from about 25 ng to about 117 ng, from about 30 ng to about 116 ng, from about 40 ng to about 115 ng, from about 50 ng to about 114 ng, from about 60 ng to about 113 ng, from about 70 ng to about 112 ng, from about 80 ng to about 111 ng, from about 90 ng to about 110 ng of the iRNA molecule, wherein the iRNA molecule encodes a live-attenuated CHIKV;
optionally the effective dose of the vaccine is selected from the group consisting of from about 1 ng to about 200,000 ng, from about 2 ng to about 150,000 ng, from about 3 ng to about 100,000 ng, from about 4 ng to about 90,00 ng, from about 5 ng to about 80,000 ng, from about 6 ng to about 70,000 ng, from about 7 ng to about 60,000 ng, from about 8 ng to about 50,000 ng, from about 9 ng to about 40,000 ng, from about 10 ng to about 30,000 ng, from about 10 ng to about 20,000 ng, from about 10 ng to about 19,000 ng, from about 10 ng to about 18,000 ng, from about 10 ng to about 17,000 ng, from about 10 ng to about 16,000 ng, from about 10 ng to about 15,000 ng, from about 10 ng to about 10,000 ng, from about 10 ng to about 5,000 ng, from about 10 ng to about 1,000 ng, from about 10 ng to about 500 ng, from about 100ng to about 500 ng, from about 100 ng to about 1,000 ng, from about 500 ng to about 5,000 ng, from about 500 ng to about 10,000 ng, from about 500 ng to about 25,000 ng, from about 500 ng to about 50,000 ng, from about 500 ng to about 100,000 ng, and from about 500 ng to about 150,000 ng of the iDNA molecule, wherein the iDNA molecule encodes a live-attenuated VEEV;
optionally the effective dose of the vaccine is selected from the group consisting of from about 1 ng to about 200,000 ng, from about 2 ng to about 150,000 ng, from about 3 ng to about 100,000 ng, from about 4 ng to about 90,00 ng, from about 5 ng to about 80,000 ng, from about 6 ng to about 70,000 ng, from about 7 ng to about 60,000 ng, from about 8 ng to about 50,000 ng, from about 9 ng to about 40,000 ng, from about 10 ng to about 30,000 ng, from about 10 ng to about 20,000 ng, from about 10 ng to about 19,000 ng, from about 10 ng to about 18,000 ng, from about 10 ng to about 17,000 ng, from about 10 ng to about 16,000 ng, from about 10 ng to about 15,000 ng, from about 10 ng to about 10,000 ng, from about 10 ng to about 5,000 ng, from about 10 ng to about 1,000 ng, from about 10 ng to about 500 ng, from about 100 ng to about 500 ng, from about 100 ng to about 1,000 ng, from about 500 ng to about 5,000 ng, from about 500 ng to about 10,000 ng, from about 500 ng to about 25,000 ng, from about 500 ng to about 50,000 ng, from about 500 ng to about 100,000 ng, and from about 500 ng to about 150,000 ng of the iRNA molecule, wherein the iRNA molecule encodes a live-attenuated VEEV;
optionally the effective dose has a concentration selected from the group consisting of from about 103 PFU to 107 PFU in 20 ul, from about 5×103 PFU to 5×106 PFU in 20 ul, from about 104 PFU to 106 PFU in 20 ul, from about 5×104 PFU to 5×105 PFU in 20 ul, from about 6×104 PFU to 4×105 PFU in 20 ul, from about 733 104 PFU to 3×105 PFU in 20 ul, from about 8×104 PFU to 2×105 PFU in 20 ul, from about 9×104 PFU to 1×105 PFU in 20 ul, and about 1×105 PFU in 20 ul of the infectious genetic material;
optionally the effective dose is selected from the group consisting of from about 10 PFU to about 10,000 PFU, from about 10 PFU to about 5,000 PFU, from about 10 PFU to about 3,100 PFU, from about 10 PFU to about 3,000 PFU, from about 10 PFU to about 2,500 PFU, from about 50 PFU to about 5,000 PFU, from about 50 PFU to about 3,100 PFU, from about 75 PFU to about 2,500 PFU, from about 100 PFU to about 1,000 PFU, from about 200 PFU to about 800 PFU, from about 300 PFU to about 700 PFU, from about 400 PFU to about 600 PFU, from about 500 PFU of the infectious genetic material;
optionally an effective amount of a single dose of the vaccine is capable providing a therapeutic benefit to a subject in need thereof;
optionally the vaccine has a low adverse reaction in a subject in need of the vaccine;
optionally the infectious genetic material is used to reduce or substantially minimize a viral infection and/or diminish the symptoms of a viral infection;
optionally the symptoms is selected from the group consisting of a fever, headache, rash, nausea, myalgia, arthralgia, respiratory failure, cardiovascular disease, hepatitis, cutaneous effects, central nervous system abnormalities, and combinations thereof;
optionally the vaccine has a low adverse reaction in a subject in need of the vaccine;
optionally the vaccine is administered by injection, including intramuscular injection and/or subcutis injection; by microinjection, including transdermal microinjection; and/or combinations thereof; and
optionally the vaccine is administered by microneedles injection.

8. An vaccine comprising the attenuated virus of claim 6 and a non-toxic pharmacological excipient thereof;
optionally the pharmacological excipient comprise a preservative and/or a saline; and optionally the saline is a phosphate buffered solution;
optionally the vaccine further comprises an adjuvant;
optionally the vaccine is a multivalent vaccine;
optionally the vaccine comprise an effective amount the attenuated virus;
optionally the effective dose has a concentration selected from the group consisting of from about 103 PFU to 107 PFU in 20 ul, from about 5×103 PFU to 5×106 PFU in 20 ul, from about 104 PFU to 106 PFU in 20 ul, from about 5×104 PFU to 5×105 PFU in 20 ul, from about 6×104 PFU to 4×105 PFU in 20 ul, from about 7×104 PFU to 3×105 PFU in 20 ul, from about 8×104 PFU to 2×105 PFU in 20 ul, from about 9×104 PFU to 1×105 PFU in 20 ul, and about 1×105 PFU in 20 ul of the attenuated virus;
optionally the effective dose is selected from the group consisting of from about 1 PFU to about 100,000 PFU, from about 2 PFU to about 100,000 PFU, from about 5 PFU to about 50,000 PFU, from about 10 PFU to about 10,000 PFU, from about 10 PFU to about 5,000 PFU, from about 10 PFU to about 3,100 PFU, from about 10 PFU to about 3,000 PFU, from about 10 PFU to about 2,500 PFU, from about 50 PFU to about 5,000 PFU, from about 60 PFU to about 2,500 PFU, from about 70 PFU to about 1,500 PFU, from about 75 PFU to about 1,300 PFU, from about 80 PFU to about 1,200 PFU, from about 90 PFU to about 1,100 PFU, from about 100 PFU to about 1,000 PFU, from about 200 PFU to about 800 PFU, from about 300 PFU to about 700 PFU, from about 400 PFU to about 600 PFU, from about 500 PFU, from about 50,000 PFU to about 500,000 PFU, from about 10,000 PFU to about 100,000 PFU, from about 5,000 PFU to about 50,000 PFU, from about 2,500 PFU to about 25,000 PFU, and about 10,000 PFU of the attenuated virus;
optionally the effective dose is selected from the group consisting of from about from about 500,000 to about 5,000,000, from about 100,000 to about 1,000,000, from about 50,000 to about 500,000, from about 25,000 to about 250,000, and about 100,000 of the attenuated virus;
optionally an effective amount of a single dose of the vaccine is capable providing a therapeutic benefit to a subject in need thereof;
optionally the vaccine has a low adverse reaction in a subject in need of the vaccine;
optionally the infectious genetic material is used to reduce or substantially minimize a viral infection and/or diminish the symptoms of a viral infection;
optionally the symptoms is selected from the group consisting of a fever, headache, rash, nausea, myalgia, arthralgia, respiratory failure, cardiovascular disease, hepatitis, cutaneous effects, central nervous system abnormalities, and/or combinations thereof;
optionally the vaccine has a low adverse reaction in a subject in need of the vaccine;
optionally the vaccine is administered by injection, including intramuscular injection and/or subcutis injection; by microinjection, including transdermal microinjection; and/or combinations thereof; and
optionally the vaccine is administered by microneedles injection.

9. A method of reducing and/or substantially minimizing a viral infection comprising directly administering an effective amount of the iRNA molecule of claim 1, the iDNA molecule of claim 3, the composition of claim 4, the vehicle is as in claims 5, the attenuated virus of claim 6, the vaccine of claim 7, and/or combinations thereof, in a subject in need thereof.

10. A method of reducing and/or substantially minimizing a viral infection comprising directly administering an effective amount of the iRNA molecule of claim 1, the iDNA molecule of claim 3, the composition of claim 4, the vehicle is as in claims 5, the attenuated virus of claim 6, the vaccine of claim 7, and/or combinations thereof, in a subject in need thereof.

11. A method of making the iDNA molecule of claim 3, the method comprising cloning a stably attenuated RNA virus into a plasmid.

12. A method of making the composition of claim 4, the method comprising mixing the iRNA of claim 1 with a pharmaceutically acceptable non-toxic component.

13. A method of making the vehicle of claim 5, the method comprising mixing the iRNA of claim 1, the iDNA molecule of claim 3, the composition of claim 4, and/or combinations thereof with a lipid and an emulsifier.

14. A method of making the virus of claim 6, the method

12. g transfecting an eukaryotic cell with the iDNA molecule of claim 12.

15. A method of making the vaccine of claim 7, the method comprising mixing the iRNA molecule of claim 1, the iDNA molecule of claim 3, the composition of claim 4, the vehicle is as in claims 5, the attenuated virus of claim 6, and/or combinations thereof;
and a non-toxic pharmacological excipient thereof.

16. A method of making a multivalent vaccine, the method comprising mixing one or more the iRNA molecule of claim 1,

3. A molecule of claim 3, the composition of claim 4, the vehicle is as in claims 5, the attenuated virus of claim 6, and/or combinations thereof; and a non-toxic pharmacological excipient thereof; wherein the one or more live attenuated virus encoded in the iRNA and/or iDNA is different.


SEQUENCE LISTING (4 sequences)

SEQ ID NO: 1 - RNA, 12142 nt, Chikungunya virus
  source 1..12142: mol_type = genomic RNA
atggctgcgtgagacacacgtagcctaccagtttcttactgctctactctgcaaagcaagagattaataacccatcatggattctgtgtacgtggacatagacgctgacagcgcctttttgaaggccctgcaacgtgcgtaccccatgtttgaggtggaacctaggcaggtcacatcgaatgaccatgctaatgctagagcgttctcgcatctagccataaaactaatagagcaggaaattgatcccgactcaaccatcctggatataggtagtgcgccagcaaggaggatgatgtcggacaggaagtaccactgcgtttgcccgatgcgcagcgcagaagatcccgagagactcgctaattatgcgagaaagctcgcatctgccgcaggaaaagtcctggacagaaacatttctggaaagatcggggacttacaagcggtgatggccgtgccagacacggagacgccaacattttgcttacacacagatgtctcatgtagacagagagcagacgtcgcgatataccaagacgtctatgctgtacacgcacccacgtcgctataccaccaggcgattaaaggagtccgagtggcgtactgggtagggttcgacacaaccccgttcatgtacaacgctatggcgggtgcctacccctcatactcgacaaattgggcggatgagcaggtactgaaggctaagaacataggattatgttcaacagacctgacggaaggtagacgaggcaaattgtctatcatgagagggaaaaagctaaaaccgtgcgaccgtgtgctgttctcagtagggtcaacgctttacccggaaagccgcacgctacttaagagctggcacctaccatcggtgttccatctaaagggcaagcttagcttcacatgccgctgtgacacagtggtttcgtgtgagggctacgtcgttaagagaataacgatgagcccaggcctttatggaaaaaccatagggtatgcggtaacccaccacgcagacggattcttgatgtgcaagactaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagaggatagtggttaacggcagaacgcaacggaacacgaacaccatgaagaactacctacttcccgtggtcgcccaggccttcagtaagtgggcaaaggagtgccggaaggacatggaagatgagaagcttctgggggtcagagaaagaacactaacctgctgctgtctatgggcatttaagaagcagaaaacacacacggtctacaagaggcctgatacccagtcaatccagaaggttcaggccgaatttgacagctttgtagtaccgggcctgtggtcgtccgggttgtcaatcccgttgaggactagaatcaagtggttgttacgcaaggtgccgaaaacagacctgatcccatacagcgggaatgcccaagaagcccaggatgcagaaaaagaagcagaggaagaacgagaagcagaactgactcatgaggctctaccacccctacaggcagcacaggaagatgtccaggtcgaaatcgacgtggaacagcttgaggatagagctggtgctggaataatagagactccgagaggcgctatcaaagttactgcccaactaacagaccacgtcgtgggggagtacctggtactttccccgcagaccgtactacgcagccagaagctcagcctgatccacgctttagcggagcaagtgaagacgtgtacgcacagcggacgagcagggaggtatgcggtcgaagcgtacgatggccgagtcctagtgccctcaggctatgcaatttcgcctgaagacttccagagtctaagcgaaagcgcaacgatggtgtacaacgaaagagagttcgtaaacagaaagttacaccacattgcgatgcacggaccagccctgaacactgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatacgaagggctaaaaattcgccccgcctgcccatacaaaattgcagtcataggagtcttcggggtaccaggatctggcaagtcagccattatcaagaacctagttaccaggcaagacctggtgactagcggaaagaaagaaaactgccaagaaatcagcaccgacgtgatgagacagagaggtctagagatatctgcacgtacggtagattcgctgctcttgaatggatgcaacagaccagtcgacgtgttgtacgtagacgaggcgtttgcgtgccactctggaacgttacttgctttgatcgccttggtgagaccaagacagaaagttgtactttgtggtgacccgaagcagtgcggcttcttcaatatgatgcagatgaaagtcaactacaatcataacatctgcacccaagtgtaccacaaaagtatctccaggcggtgtacactgcctgtgactgccattgtgtcatcgttgcattacgaaggcaaaatgcgcactacgaatgagtacaacatgccgattgtagtggacactacaggctcaacgaaacctgaccctggagacctcgtgttaacgtgcttcagagggtgggttaaacaactgcaaattgactatcgtggacacgaggtcatgacagcagccgcatcccaagggttaactagaaaaggagtttacgcagttaggcaaaaagttaacgaaaacccactctatgcatcaacatcagagcacgtcaacgtactcctaacgcgtacggaaggtaaactggtatggaagacactctctggtgacccgtggataaagacgctgcagaacccaccgaaaggaaacttcaaagcaactattaaggagtgggaggtggagcacgcatcgataatggcgggcatctgcagtcaccaagtgacctttgacacattccaaaacaaagccaacgtttgctgggctaagagcttggtccctatcctcgaaacagcggggataaaactaaatgataggcagtggtcccagataattcaagccttcaaagaagacaaagcatactcacccgaagtagccctgaatgaaatatgcacgcgcatgtatggggtggatctagacagtgggctattctctaaaccgttggtatctgtgtattacgcggataaccattgggataataggccgggaggaaagatgttcggattcaaccctgaggcagcgtccattctagaaagaaagtacccatttacaaaaggaaagtggaacatcaacaagcagatctgcgtgactaccaggaggatagaagacttcaaccctaccaccaacattataccggtcaacaggagactaccacactcattagtggccgaacaccgcccagtaaaaggggaaagaatggaatggctggttaacaagataaacggacaccacgtactcctggttagcggctataaccttgcactgcctactaagagagtcacctgggtagcgccactaggtgtccgcggagcggactatacatacaacctagagctgggtctaccagcaacrcttggtaggtatgacctagtggtcataaacatccacacaccttttcgcatacaccattaccaacagtgcgtagatcacgcaatgaaactgcaaatgctagggggtgactcactgagactgctcaaaccgggtggctctctattgatcagagcatacggttacgcagatagaaccagtgaacgagtcatctgcgtactgggacgcaagtttagatcgtctagagcattgaaaccaccatgtgtcaccagtaatactgagatgtttttcctatttagcaattttgacaatggcagaaggaattttacaacgcatgtcatgaacaatcaactgaatgcagcctttgtaggacaggccacccgagcaggatgtgcaccatcgtaccgggtaaaacgcatggacatcgcgaagaacgatgaagagtgcgtggttaacgccgccaaccctcgcgggttaccaggtgacggtgtttgcaaggcagtatataaaaagtggccggagtcctttaaaaacagtgcaacaccagtaggaaccgcaaaaacagttatgtgcggtacgtatccagtaatccacgccgtaggaccaaacttctcaaattattcggagtctgaaggggaccgggaattggcggctgcctatcgagaagtcgcaaaggaagtaactagactgggagtaaatagcgtagctatacctctcctctccacaggtgtatactcaggagggaaagacaggctaacccagtcactgaaccacctctttacagccatggactcgacggatgcagacgtggtcatctactgccgagacaaggaatgggagaagaaaatatctgaggccatacagatgcggacccaagtggagctgctggatgagcacatctccatagactgcgatgtcattcgcgtgcaccctgacagtagcttggcaggcagaaaaggatacagcaccacggaaggcgcactgtattcatatctagaagggacacgttttcaccagacggcagtggatatggcagagatatacactatgtggccaaagcaaacagaggccaatgagcaagtctgcctatatgccctgggggaaagtattgaatcaatcaggcagaaatgcccggtggatgatgcagacgcatcatctcccccgaaaactgtcccgtgtctttgccggtatgccatgactcctgaacgcgtcacccgacttcgcatgaaccatgtcacaaatataattgtgtgttcttcatttccccttccaaagtacaagatagaaggagtgcaaaaagtcaaatgctccaaggtaatgttattcgatcacaatgtgccatcgcgcgtaagtccaagggaatacagatcttcccaggagtctgtacaggaagtgagtacgacaacgtcattgacgcatagccagtttgatctaagcgccgatggcgagacactgcctgtcccgtcagacctggatgctgacgccccagccctagaaccggccctagacgacggggcggtacatacattaccaaccataatcggaaaccttgcggccgtgtctgactgggtaatgagcaccgtacctgtcgcgccgcctagaagaaggagagggagaaacctgactgtgacatgtgacgagagagaagggaatataacacccatggctagcgtccgattctttagagcagagctgtgtccggccgtacaagaaacagcggagacgcgtgacacagctatttcccttcaggcaccgccaagtaccaccatggaactgagccatccaccgatctccttcggagcaccaagcgagacgttccccatcacatttggggacttcgacgaaggagaaatcgaaagcttgtcttctgagctactaactttcggagacttcctacccggtgaagtggatgatctgacagatagcgactggtccacgtgcccagacacggacgacgagttatgactagacagggcaggtgggtatatattctcgtcggacactggtccaggccatttacaacagaagtcggtacgccagtcagtgctgccggtaaacaccctggaggaagtccacgaggagaagtgttacccacctaagctggatgaattaaaggagcaactactacttaagaaactccaggagagtgcgtccatggccaatagaagcaggtatcagtcacgcaaagtggaaaatatgaaagcaacaatcatccagagactaaagagaggctgtaaactgtatttaatggcagagaccccgaaagtcccgacttatcggaccatatacccggcgcctgtgtactcgcctccgatcaatgtccgattgtccaaccccgagtccgcagtggcagcatgtaatgagttcttagctagaaactacccaactgtttcatcataccaaatcaccgacgagtatgatgcatatctagacatggtggacgggtcggagagttgcttggaccgagcgacattcaatccgtcaaaacttaggagctacccgaaacaacatgcttatcacgcgccttctatcagaagcgctgtaccttccccattccagaacacactacagaatgtactggcagcagccacgaaaaggaactgcaacgtcacacagatgagggaattacccactttggactcagcagtattcaacgtggagtgttttaaaaaattcgcatgtaaccgagaatactgggaagaatttgcagccagccctatcaggataacaactgagaatctaacaacctatgtcactaaactaaaggggccaaaagcagcagcgctgtttgcaaaaacccataatctgctgccactgcaggatgtaccaatggataggttcacagtagatatgaaaagggatgtgaaggtaactcctggtacaaagcatacagaggaaagacctaaggtgcaggttatacaggcggctgaacccttggcaacagcgtacctatgtggaattcacagagaactggttaggagattgaacgccgtcctcctacccaatgtgcatacactatttgacatgtctgccgaggacttcgatgccattatagccgcacacttcaagccaggagacgctgttttagaaacggacatagcctcctttgataagagccaagatgattcacttgcgcttaccgccttaatgctgttagaagatttgggagtggatcactccctgttggacttgatagaggctgctttcggagagatttccagctgtcatctgccgacaggtacgcgcttcaagttcggcgctatgatgaaatccggtatgttcctaactctgttcgtcaacacgttgttaaatatcaccatcgctagccgggtgttggaagatcgtctgacaaaatccgcatgcgcggccttcatcggcgacgacaacataatacatggtgtcgtctccgatgaattgatggcagccagatgcgctacttggatgaacatggaagtgaagatcatagatgcagttgtatcccagaaagctccttacttttgtggagggtttatactgcatgatactgtgacaggaacagcttgcagagtggcggacccgctaaaaaggttatttaaattgggcaaaccgttagcggcaggtgacgaacaagatgaagacagaagacgggcgctggctgatgaagtaatcagatggcaacgaacagggctaatagatgagctggagaaagcggtgtactctaggtacgaagtgcagggtatatcagttgcggtaatgtccatggccacctttgcaagctccagatccaacttcgagaagctcagaggacccgtcataactttgtacggcggtcctaaataggtacgcactacagctacctattttgcagaagccgacagcaggtacctaaataccaatcagccataatgagtcttgccattccagttatgtgcctgctggcaaataccacgttcccctgctcccagcccccttgcacaccctgctgctacgaaaaagagccggagaaaaccctgcgcatgctagaagacaacgtcatgagccccgggtactatcagctgctacaagcatccttaacatgttctccccgccgccagcgacgcagtattaaggacaacttcaatgtctataaagccataagaccgtacctagctcactgtcccgactgtggagaagggcactcgtgccatagtcccgtagcgctagaacgcatcagaaacgaagcgacagacgggacgctgaaaatccaggtttccttgcaaatcggaataaagacggatgatagccatgattggaccaagctgcgttacatggacaatcatatgccagcagacgcagagagggccaggctatttgtaagaacgtcagcaccgtgcacgattactggaacaatgggacacttcatcctggcccgatgtccgaaaggagaaactctgacggtgggattcactgacggtaggaagatcagtcactcatgtacgcacccatttcaccacgaccctcctgtgataggccgggaaaaatttcattcccgaccgcagcacggtagagaactaccttgcagcacgtacgcgcagagcaccgctgcaactgccgaggagatagaggtacatatgcccccagacaccccagatcgcacattgatgtcacaacagtccggtaatgtaaagatcacagtcaatagtcagacggtgcggtacaagtgtaattgcggtgactcaaatgaaggactaaccactacagacaaagtgattaataactgcaaggttgatcaatgccatgccgcggtcaccaatcacaaaaaatggcagtataattcccctctggtcccgcgtaatgctgaactcggggaccgaaaaggaaaagttcacattccgtttcctctggcaaatgtgacatgcagggtgcctaaggcaaggaaccccaccgtgacgtacggaaaaaaccaagtcatcatgctgctgtatcctgaccacccaacgctcctgtcctaccggaatatgggagaagaaccaaactatcaagaagagtgggtgacgcataagaaggagatcaggttaaccgtgccgactgaagggctcgaggtcacgtggggcaacaacgagccgtacaagtattggccgcagttatccacaaacggtacagcccacggccacccgcatgagataattttgtattattatgagctgtaccctactatgactgtggtagttgtgtcagtggcctcgttcgtactcctgtcgatggtgggtgtggcagtggggatgtgcatgtgtgcacgacgcagatgcattacaccgtacgaactgacaccaggagctaccgtccctttcctgcttagcctaatatgctgcattagaacagctaaagcggccacataccaagaggctgcggtatacctgtggaacgagcagcagcctttgttttggctgcaagcccttattccgctggcagccctgattgtcctatgcaactgtctgagactcttaccatgcttttgtaaaacgttgacttttttagccgtaatgagcgtcggtgcccacactgtgagcgcgtacgaacacgtaacagtgatcccgaacacggtgggagtaccgtataagactctagtcaacagaccgggctacagccccatggtactggagatggagcttctgtcagtcactttggagccaacgctatcgcttgattacatcacgtgcgagtataaaaccgtcatcccgtctccgtacgtgaaatgctgcggtacagcagagtgcaaggacaagagcctacctgattacagctgtaaggtcttcaccggcgtctacccattcatgtggggcggcgcctactgcttctgcgacactgaaaatacgcaattgagcgaagcacatgtggagaagtccgaatcatgcaaaacagaatttgcatcagcatatagggctcataccgcatccgcatcagctaagctccgcgtcctttaccaaggaaataatgttactgtatctgcttatgcaaacggcgatcatgccgtcacagttaaggacgctaaattcattgtggggccaatgtcttcagcctggacaccttttgacaataaaatcgtggtgtacaaaggcgacgtctacaacatggactacccgcccttcggcgcaggaagaccaggacaatttggcgacatccaaagtcgcacgcctgagagcgaagacgtctatgctaacacacaactggtactgcagagaccgtccgcgggtacggtgcacgtgccgtactctcaggcaccatctggcttcaagtattggctaaaagaacgaggggcgtcgctgcagcacacagcaccatttggctgtcaaatagcaacaaacccggtaagagcgatgaactgcgccgtagggaacatgcctatctccatcgacataccggacgcggccttcactagggtcgtcgacgcgccatctttaacggacatgtcgtgtgaggtaccagcctgcacccactcctcagactttgggggcgtagccatcattaaatatgcagccagcaagaaaggcaagtgtgcggtgcattcgatgactaacgccgtcactattcgggaagctgaaatagaagtagaagggaactctcagttgcaaatctctttttcgacggccctagccagcgccgaattccgcgtacaagtctgttctacacaagtacactgtgcagccgagtgccatccaccgaaagaccatatagtcaattacccggcgtcacacaccaccctcggggtccaagacatttccgttacggcgatgtcatgggtgcagaagatcacgggaggtgtgggactggttgtcgctgttgcagcactgatcctaatcgtggtgctatgcgtgtcgtttagcaggcactaagacccgtcataactttgtacggcggtcctaaataggtacgcactacagctacctattttgcagaagccgacagcaggtacctaaataccaatcagccataatggagtttatcccaacccaaactttctacaataggaggtaccagcctcgaccttggactccgcgccctactatccaagttatcagacccagaccgcgtccgcaaaggaaagccgggcaacttgcccagctgatctcagcagttaataaactgacaatgcgcgcggtacctcaacagaagccgcgcaagaatcggaagaataagaagcaaaagcaaaagcagcaggcgccacgaaacaacatgaatcaaaagaagcagccccctaaaaagaaaccggctcaaaagaaaaagaagccgggccgtagagagagaatgtgcatgaaaatcgaaaatgattgcatcttcgaagtcaagcatgaaggtaaggtaacaggttacgcgtgcttggtaggggacaaagtaatgaagccagcacacgtaaaggggaccatcgataatgcggacctggccaaattggccttcaagcggtcatctaagtacgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgcgggcaaaccaggggacagcggtagaccgatcttcgacaacaaggggcgcgtggtggccatagttttaggaggagctaatgaaggagcccgtacagccctctcggtggtgacctggaacaaagacatcgtcacgaaaatcacccctgagggggccgaagagtggtgacttgacaactaggtacgaaggtatatgtgtcccctaagagacacaccacatatagctaagaatcaatagataagtatagatcaaagggctgaacaacccctgaatagtaacaaaatataaaaatcaacaaaaatcataaaatagaaaaccagaaacagaagtaggtaagaaggtatatgtgtcccctaagagacacaccatatatagctaagaatcaatagataagtatagatcaaagggctgaataacccctgaataataacaaaatataaaaatcaataaaaatcataaaatagaaaaccataaacagaagtagttcaaagggctataaaacccctgaatagtaacaaaacataaaactaataaaaatcaaatgaataccataattggcaatcggaagagatgtaggtacttaagcttcctaaaagcagccgaactcgctttgagatgtaggcgtagcacaccgaactcttccataattctccgaacccacagggacgtaggagatgttcaaagtggctataaaaccctgaacagtaataaaacataaaattaataaggatcaaatgagtaccataattggcaaacggaagagatgtaggtacttaagcttcctaaaagcagccgaactcactttgagatgtaggcatagcataccgaactcttccacaattctccgtacccatagggacgtaggagatgttattttgtttttaatatttc

SEQ ID NO: 2 - DNA, 12161 bp, Chikungunya virus
  source 1..12161: mol_type = genomic DNA
ctaatacgactcactatagatggctgcgtgagacacacgtagcctaccagtttcttactgctctactctgcaaagcaagagattaataacccatcatggattctgtgtacgtggacatagacgctgacagcgcctttttgaaggccctgcaacgtgcgtaccccatgtttgaggtggaacctaggcaggtcacatcgaatgaccatgctaatgctagagcgttctcgcatctagccataaaactaatagagcaggaaattgatcccgactcaaccatcctggatataggtagtgcgccagcaaggaggatgatgtcggacaggaagtaccactgcgtttgcccgatgcgcagcgcagaagatcccgagagactcgctaattatgcgagaaagctcgcatctgccgcaggaaaagtcctggacagaaacatttctggaaagatcggggacttacaagcggtgatggccgtgccagacacggagacgccaacattttgcttacacacagatgtctcatgtagacagagagcagacgtcgcgatataccaagacgtctatgctgtacacgcacccacgtcgctataccaccaggcgattaaaggagtccgagtggcgtactgggtagggttcgacacaaccccgttcatgtacaacgctatggcgggtgcctacccctcatactcgacaaattgggcggatgagcaggtactgaaggctaagaacataggattatgttcaacagacctgacggaaggtagacgaggcaaattgtctatcatgagagggaaaaagctaaaaccgtgcgaccgtgtgctgttctcagtagggtcaacgctttacccggaaagccgcacgctacttaagagctggcacctaccatcggtgttccatctaaagggcaagcttagcttcacatgccgctgtgacacagtggtttcgtgtgagggctacgtcgttaagagaataacgatgagcccaggcctttatggaaaaaccatagggtatgcggtaacccaccacgcagacggattcttgatgtgcaagactaccgacacggttgacggcgaaagagtgtcattctcggtgtgcacgtacgtgccggcgaccatttgtgatcaaatgaccggcatccttgctacagaagtcacgccggaggatgcacagaagctgttggtggggctgaaccagaggatagtggttaacggcagaacgcaacggaacacgaacaccatgaagaactacctacttcccgtggtcgcccaggccttcagtaagtgggcaaaggagtgccggaaggacatggaagatgagaagcttctgggggtcagagaaagaacactaacctgctgctgtctatgggcatttaagaagcagaaaacacacacggtctacaagaggcctgatacccagtcaatccagaaggttcaggccgaatttgacagctttgtagtaccgggcctgtggtcgtccgggttgtcaatcccgttgaggactagaatcaagtggttgttacgcaaggtgccgaaaacagacctgatcccatacagcgggaatgcccaagaagcccaggatgcagaaaaagaagcagaggaagaacgagaagcagaactgactcatgaggctctaccacccctacaggcagcacaggaagatgtccaggtcgaaatcgacgtggaacagcttgaggatagagctggtgctggaataatagagactccgagaggcgctatcaaagttactgcccaactaacagaccacgtcgtgggggagtacctggtactttccccgcagaccgtactacgcagccagaagctcagcctgatccacgctttagcggagcaagtgaagacgtgtacgcacagcggacgagcagggaggtatgcggtcgaagcgtacgatggccgagtcctagtgccctcaggctatgcaatttcgcctgaagacttccagagtctaagcgaaagcgcaacgatggtgtacaacgaaagagagttcgtaaacagaaagttacaccacattgcgatgcacggaccagccctgaacactgacgaagagtcgtatgagctggtgagggcagagaggacagaacacgagtacgtctacgacgtggaccagagaagatgctgtaagaaggaagaagctgcaggactggtactggtgggcgacttgactaatccgccctaccacgaattcgcatacgaagggctaaaaattcgccccgcctgcccatacaaaattgcagtcataggagtcttcggggtaccaggatctggcaagtcagccattatcaagaacctagttaccaggcaagacctggtgactagcggaaagaaagaaaactgccaagaaatcagcaccgacgtgatgagacagagaggtctagagatatctgcacgtacggtagattcgctgctcttgaatggatgcaacagaccagtcgacgtgttgtacgtagacgaggcgtttgcgtgccactctggaacgttacttgctttgatcgccttggtgagaccaagacagaaagttgtactttgtggtgacccgaagcagtgcggcttcttcaatatgatgcagatgaaagtcaactacaatcataacatctgcacccaagtgtaccacaaaagtatctccaggcggtgtacactgcctgtgactgccattgtgtcatcgttgcattacgaaggcaaaatgcgcactacgaatgagtacaacatgccgattgtagtggacactacaggctcaacgaaacctgaccctggagacctcgtgttaacgtgcttcagagggtgggttaaacaactgcaaattgactatcgtggacacgaggtcatgacagcagccgcatcccaagggttaactagaaaaggagtttacgcagttaggcaaaaagttaacgaaaacccactctatgcatcaacatcagagcacgtcaacgtactcctaacgcgtacggaaggtaaactggtatggaagacactctctggtgacccgtggataaagacgctgcagaacccaccgaaaggaaacttcaaagcaactattaaggagtgggaggtggagcacgcatcgataatggcgggcatctgcagtcaccaagtgacctttgacacattccaaaacaaagccaacgtttgctgggctaagagcttggtccctatcctcgaaacagcggggataaaactaaatgataggcagtggtcccagataattcaagccttcaaagaagacaaagcatactcacccgaagtagccctgaatgaaatatgcacgcgcatgtatggggtggatctagacagtgggctattctctaaaccgttggtatctgtgtattacgcggataaccattgggataataggccgggaggaaagatgttcggattcaaccctgaggcagcgtccattctagaaagaaagtacccatttacaaaaggaaagtggaacatcaacaagcagatctgcgtgactaccaggaggatagaagacttcaaccctaccaccaacattataccggtcaacaggagactaccacactcattagtggccgaacaccgcccagtaaaaggggaaagaatggaatggctggttaacaagataaacggacaccacgtactcctggttagcggctataaccttgcactgcctactaagagagtcacctgggtagcgccactaggtgtccgcggagcggactatacatacaacctagagctgggtctaccagcaacrcttggtaggtatgacctagtggtcataaacatccacacaccttttcgcatacaccattaccaacagtgcgtagatcacgcaatgaaactgcaaatgctagggggtgactcactgagactgctcaaaccgggtggctctctattgatcagagcatacggttacgcagatagaaccagtgaacgagtcatctgcgtactgggacgcaagtttagatcgtctagagcattgaaaccaccatgtgtcaccagtaatactgagatgtttttcctatttagcaattttgacaatggcagaaggaattttacaacgcatgtcatgaacaatcaactgaatgcagcctttgtaggacaggccacccgagcaggatgtgcaccatcgtaccgggtaaaacgcatggacatcgcgaagaacgatgaagagtgcgtggttaacgccgccaaccctcgcgggttaccaggtgacggtgtttgcaaggcagtatataaaaagtggccggagtcctttaaaaacagtgcaacaccagtaggaaccgcaaaaacagttatgtgcggtacgtatccagtaatccacgccgtaggaccaaacttctcaaattattcggagtctgaaggggaccgggaattggcggctgcctatcgagaagtcgcaaaggaagtaactagactgggagtaaatagcgtagctatacctctcctctccacaggtgtatactcaggagggaaagacaggctaacccagtcactgaaccacctctttacagccatggactcgacggatgcagacgtggtcatctactgccgagacaaggaatgggagaagaaaatatctgaggccatacagatgcggacccaagtggagctgctggatgagcacatctccatagactgcgatgtcattcgcgtgcaccctgacagtagcttggcaggcagaaaaggatacagcaccacggaaggcgcactgtattcatatctagaagggacacgttttcaccagacggcagtggatatggcagagatatacactatgtggccaaagcaaacagaggccaatgagcaagtctgcctatatgccctgggggaaagtattgaatcaatcaggcagaaatgcccggtggatgatgcagacgcatcatctcccccgaaaactgtcccgtgtctttgccggtatgccatgactcctgaacgcgtcacccgacttcgcatgaaccatgtcacaaatataattgtgtgttcttcatttccccttccaaagtacaagatagaaggagtgcaaaaagtcaaatgctccaaggtaatgttattcgatcacaatgtgccatcgcgcgtaagtccaagggaatacagatcttcccaggagtctgtacaggaagtgagtacgacaacgtcattgacgcatagccagtttgatctaagcgccgatggcgagacactgcctgtcccgtcagacctggatgctgacgccccagccctagaaccggccctagacgacggggcggtacatacattaccaaccataatcggaaaccttgcggccgtgtctgactgggtaatgagcaccgtacctgtcgcgccgcctagaagaaggagagggagaaacctgactgtgacatgtgacgagagagaagggaatataacacccatggctagcgtccgattctttagagcagagctgtgtccggccgtacaagaaacagcggagacgcgtgacacagctatttcccttcaggcaccgccaagtaccaccatggaactgagccatccaccgatctccttcggagcaccaagcgagacgttccccatcacatttggggacttcgacgaaggagaaatcgaaagcttgtcttctgagctactaactttcggagacttcctacccggtgaagtggatgatctgacagatagcgactggtccacgtgcccagacacggacgacgagttatgactagacagggcaggtgggtatatattctcgtcggacactggtccaggccatttacaacagaagtcggtacgccagtcagtgctgccggtaaacaccctggaggaagtccacgaggagaagtgttacccacctaagctggatgaattaaaggagcaactactacttaagaaactccaggagagtgcgtccatggccaatagaagcaggtatcagtcacgcaaagtggaaaatatgaaagcaacaatcatccagagactaaagagaggctgtaaactgtatttaatggcagagaccccgaaagtcccgacttatcggaccatatacccggcgcctgtgtactcgcctccgatcaatgtccgattgtccaaccccgagtccgcagtggcagcatgtaatgagttcttagctagaaactacccaactgtttcatcataccaaatcaccgacgagtatgatgcatatctagacatggtggacgggtcggagagttgcttggaccgagcgacattcaatccgtcaaaacttaggagctacccgaaacaacatgcttatcacgcgccttctatcagaagcgctgtaccttccccattccagaacacactacagaatgtactggcagcagccacgaaaaggaactgcaacgtcacacagatgagggaattacccactttggactcagcagtattcaacgtggagtgttttaaaaaattcgcatgtaaccgagaatactgggaagaatttgcagccagccctatcaggataacaactgagaatctaacaacctatgtcactaaactaaaggggccaaaagcagcagcgctgtttgcaaaaacccataatctgctgccactgcaggatgtaccaatggataggttcacagtagatatgaaaagggatgtgaaggtaactcctggtacaaagcatacagaggaaagacctaaggtgcaggttatacaggcggctgaacccttggcaacagcgtacctatgtggaattcacagagaactggttaggagattgaacgccgtcctcctacccaatgtgcatacactatttgacatgtctgccgaggacttcgatgccattatagccgcacacttcaagccaggagacgctgttttagaaacggacatagcctcctttgataagagccaagatgattcacttgcgcttaccgccttaatgctgttagaagatttgggagtggatcactccctgttggacttgatagaggctgctttcggagagatttccagctgtcatctgccgacaggtacgcgcttcaagttcggcgctatgatgaaatccggtatgttcctaactctgttcgtcaacacgttgttaaatatcaccatcgctagccgggtgttggaagatcgtctgacaaaatccgcatgcgcggccttcatcggcgacgacaacataatacatggtgtcgtctccgatgaattgatggcagccagatgcgctacttggatgaacatggaagtgaagatcatagatgcagttgtatcccagaaagctccttacttttgtggagggtttatactgcatgatactgtgacaggaacagcttgcagagtggcggacccgctaaaaaggttatttaaattgggcaaaccgttagcggcaggtgacgaacaagatgaagacagaagacgggcgctggctgatgaagtaatcagatggcaacgaacagggctaatagatgagctggagaaagcggtgtactctaggtacgaagtgcagggtatatcagttgcggtaatgtccatggccacctttgcaagctccagatccaacttcgagaagctcagaggacccgtcataactttgtacggcggtcctaaataggtacgcactacagctacctattttgcagaagccgacagcaggtacctaaataccaatcagccataatgagtcttgccattccagttatgtgcctgctggcaaataccacgttcccctgctcccagcccccttgcacaccctgctgctacgaaaaagagccggagaaaaccctgcgcatgctagaagacaacgtcatgagccccgggtactatcagctgctacaagcatccttaacatgttctccccgccgccagcgacgcagtattaaggacaacttcaatgtctataaagccataagaccgtacctagctcactgtcccgactgtggagaagggcactcgtgccatagtcccgtagcgctagaacgcatcagaaacgaagcgacagacgggacgctgaaaatccaggtttccttgcaaatcggaataaagacggatgatagccatgattggaccaagctgcgttacatggacaatcatatgccagcagacgcagagagggccaggctatttgtaagaacgtcagcaccgtgcacgattactggaacaatgggacacttcatcctggcccgatgtccgaaaggagaaactctgacggtgggattcactgacggtaggaagatcagtcactcatgtacgcacccatttcaccacgaccctcctgtgataggccgggaaaaatttcattcccgaccgcagcacggtagagaactaccttgcagcacgtacgcgcagagcaccgctgcaactgccgaggagatagaggtacatatgcccccagacaccccagatcgcacattgatgtcacaacagtccggtaatgtaaagatcacagtcaatagtcagacggtgcggtacaagtgtaattgcggtgactcaaatgaaggactaaccactacagacaaagtgattaataactgcaaggttgatcaatgccatgccgcggtcaccaatcacaaaaaatggcagtataattcccctctggtcccgcgtaatgctgaactcggggaccgaaaaggaaaagttcacattccgtttcctctggcaaatgtgacatgcagggtgcctaaggcaaggaaccccaccgtgacgtacggaaaaaaccaagtcatcatgctgctgtatcctgaccacccaacgctcctgtcctaccggaatatgggagaagaaccaaactatcaagaagagtgggtgacgcataagaaggagatcaggttaaccgtgccgactgaagggctcgaggtcacgtggggcaacaacgagccgtacaagtattggccgcagttatccacaaacggtacagcccacggccacccgcatgagataattttgtattattatgagctgtaccctactatgactgtggtagttgtgtcagtggcctcgttcgtactcctgtcgatggtgggtgtggcagtggggatgtgcatgtgtgcacgacgcagatgcattacaccgtacgaactgacaccaggagctaccgtccctttcctgcttagcctaatatgctgcattagaacagctaaagcggccacataccaagaggctgcggtatacctgtggaacgagcagcagcctttgttttggctgcaagcccttattccgctggcagccctgattgtcctatgcaactgtctgagactcttaccatgcttttgtaaaacgttgacttttttagccgtaatgagcgtcggtgcccacactgtgagcgcgtacgaacacgtaacagtgatcccgaacacggtgggagtaccgtataagactctagtcaacagaccgggctacagccccatggtactggagatggagcttctgtcagtcactttggagccaacgctatcgcttgattacatcacgtgcgagtataaaaccgtcatcccgtctccgtacgtgaaatgctgcggtacagcagagtgcaaggacaagagcctacctgattacagctgtaaggtcttcaccggcgtctacccattcatgtggggcggcgcctactgcttctgcgacactgaaaatacgcaattgagcgaagcacatgtggagaagtccgaatcatgcaaaacagaatttgcatcagcatatagggctcataccgcatccgcatcagctaagctccgcgtcctttaccaaggaaataatgttactgtatctgcttatgcaaacggcgatcatgccgtcacagttaaggacgctaaattcattgtggggccaatgtcttcagcctggacaccttttgacaataaaatcgtggtgtacaaaggcgacgtctacaacatggactacccgcccttcggcgcaggaagaccaggacaatttggcgacatccaaagtcgcacgcctgagagcgaagacgtctatgctaacacacaactggtactgcagagaccgtccgcgggtacggtgcacgtgccgtactctcaggcaccatctggcttcaagtattggctaaaagaacgaggggcgtcgctgcagcacacagcaccatttggctgtcaaatagcaacaaacccggtaagagcgatgaactgcgccgtagggaacatgcctatctccatcgacataccggacgcggccttcactagggtcgtcgacgcgccatctttaacggacatgtcgtgtgaggtaccagcctgcacccactcctcagactttgggggcgtagccatcattaaatatgcagccagcaagaaaggcaagtgtgcggtgcattcgatgactaacgccgtcactattcgggaagctgaaatagaagtagaagggaactctcagttgcaaatctctttttcgacggccctagccagcgccgaattccgcgtacaagtctgttctacacaagtacactgtgcagccgagtgccatccaccgaaagaccatatagtcaattacccggcgtcacacaccaccctcggggtccaagacatttccgttacggcgatgtcatgggtgcagaagatcacgggaggtgtgggactggttgtcgctgttgcagcactgatcctaatcgtggtgctatgcgtgtcgtttagcaggcactaagacccgtcataactttgtacggcggtcctaaataggtacgcactacagctacctattttgcagaagccgacagcaggtacctaaataccaatcagccataatggagtttatcccaacccaaactttctacaataggaggtaccagcctcgaccttggactccgcgccctactatccaagttatcagacccagaccgcgtccgcaaaggaaagccgggcaacttgcccagctgatctcagcagttaataaactgacaatgcgcgcggtacctcaacagaagccgcgcaagaatcggaagaataagaagcaaaagcaaaagcagcaggcgccacgaaacaacatgaatcaaaagaagcagccccctaaaaagaaaccggctcaaaagaaaaagaagccgggccgtagagagagaatgtgcatgaaaatcgaaaatgattgcatcttcgaagtcaagcatgaaggtaaggtaacaggttacgcgtgcttggtaggggacaaagtaatgaagccagcacacgtaaaggggaccatcgataatgcggacctggccaaattggccttcaagcggtcatctaagtacgaccttgaatgcgcgcagatacccgtgcacatgaagtccgacgcttcgaagttcacccatgagaaaccggaggggtactacaactggcaccacggagcagtacagtactcaggaggccggttcaccatccctacaggtgcgggcaaaccaggggacagcggtagaccgatcttcgacaacaaggggcgcgtggtggccatagttttaggaggagctaatgaaggagcccgtacagccctctcggtggtgacctggaacaaagacatcgtcacgaaaatcacccctgagggggccgaagagtggtgacttgacaactaggtacgaaggtatatgtgtcccctaagagacacaccacatatagctaagaatcaatagataagtatagatcaaagggctgaacaacccctgaatagtaacaaaatataaaaatcaacaaaaatcataaaatagaaaaccagaaacagaagtaggtaagaaggtatatgtgtcccctaagagacacaccatatatagctaagaatcaatagataagtatagatcaaagggctgaataacccctgaataataacaaaatataaaaatcaataaaaatcataaaatagaaaaccataaacagaagtagttcaaagggctataaaacccctgaatagtaacaaaacataaaactaataaaaatcaaatgaataccataattggcaatcggaagagatgtaggtacttaagcttcctaaaagcagccgaactcgctttgagatgtaggcgtagcacaccgaactcttccataattctccgaacccacagggacgtaggagatgttcaaagtggctataaaaccctgaacagtaataaaacataaaattaataaggatcaaatgagtaccataattggcaaacggaagagatgtaggtacttaagcttcctaaaagcagccgaactcactttgagatgtaggcatagcataccgaactcttccacaattctccgtacccatagggacgtaggagatgttattttgtttttaatatttc

SEQ ID NO: 3 - RNA, 11530 nt, Venezuelan equine encephalitis virus
  source 1..11530: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaagttcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttctcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccctatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaatgagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactctatacatctcgcccgtgtcacattgtggatgggcacggttatttcctgcttgccaggtgcccggcaggggactccatcaccatggaatttaagaaagattccgtccgacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgatgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataaccttcggtttcagatcagtgtcactgaaactgcaccctaagaatcccacatatctaatcacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgttgaagcatataaagcgcacacagcctcagtgcaggcgttcctcaacatcacagtgggagaacactctattgtgactaccgtgtatgtgaatggagaaactcctgtgaatttcaatggggtcaaaataactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctctgatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgagtgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgacctgcaggagaatcgatgggcccctataactctctacggctaacctgaatggactacgacatagtctagtccgccaagatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtgaatacagcagcaattggcaagctgcttacatagaactcgcggcgattggcatgccgctttaaaatttttattttatttttcttttcttttccgaatcggattttgtttttaatatttc

SEQ ID NO: 4 - DNA, 11549 bp, Venezuelan equine encephalitis virus
  source 1..11549: mol_type = genomic DNA
ctaatacgactcactatagataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaagttcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttctcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccctatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtcactagtgaccaccatgtgtctgctcgccaatgtgacgttcccatgtgctcaaccaccaatttgctacgacagaaaaccagcagagactttggccatgctcagcgttaacgttgacaacccgggctacgatgagctgctggaagcagctgttaagtgccccggaaggaaaaggagatccaccgaggagctgtttaatgagtataagctaacgcgcccttacatggccagatgcatcagatgtgcagttgggagctgccatagtccaatagcaatcgaggcagtaaagagcgacgggcacgacggttatgttagacttcagacttcctcgcagtatggcctggattcctccggcaacttaaagggcaggaccatgcggtatgacatgcacgggaccattaaagagataccactacatcaagtgtcactctatacatctcgcccgtgtcacattgtggatgggcacggttatttcctgcttgccaggtgcccggcaggggactccatcaccatggaatttaagaaagattccgtccgacactcctgctcggtgccgtatgaagtgaaatttaatcctgtaggcagagaactctatactcatcccccagaacacggagtagagcaagcgtgccaagtctacgcacatgatgcacagaacagaggagcttatgtcgagatgcacctcccgggctcagaagtggacagcagtttggtttccttgagcggcagttcagtcaccgtgacacctcctgatgggactagcgccctggtggaatgcgagtgtggcggcacaaagatctccgagaccatcaacaagacaaaacagttcagccagtgcacaaagaaggagcagtgcagagcatatcggctgcagaacgataagtgggtgtataattctgacaaactgcccaaagcagcgggagccaccttaaaaggaaaactgcatgtcccattcttgctggcagacggcaaatgcaccgtgcctctagcaccagaacctatgataaccttcggtttcagatcagtgtcactgaaactgcaccctaagaatcccacatatctaatcacccgccaacttgctgatgagcctcactacacgcacgagctcatatctgaaccagctgttaggaattttaccgtcaccgaaaaagggtgggagtttgtatggggaaaccacccgccgaaaaggttttgggcacaggaaacagcacccggaaatccacatgggctaccgcacgaggtgataactcattattaccacagataccctatgtccaccatcctgggtttgtcaatttgtgccgccattgcaaccgtttccgttgcagcgtctacctggctgttttgcagatctagagttgcgtgcctaactccttaccggctaacacctaacgctaggataccattttgtctggctgtgctttgctgcgcccgcactgcccgggccgagaccacctgggagtccttggatcacctatggaacaataaccaacagatgttctggattcaattgctgatccctctggccgccttgatcgtagtgactcgcctgctcaggtgcgtgtgctgtgtcgtgccttttttagtcatggccggcgccgcaggcgccggcgcctacgagcacgcgaccacgatgccgagccaagcgggaatctcgtataacactatagtcaacagagcaggctacgcaccactccctatcagcataacaccaacaaagatcaagctgatacctacagtgaacttggagtacgtcacctgccactacaaaacaggaatggattcaccagccatcaaatgctgcggatctcaggaatgcactccaacttacaggcctgatgaacagtgcaaagtcttcacaggggtttacccgttcatgtggggtggtgcatattgcttttgcgacactgagaacacccaagtcagcaaggcctacgtaatgaaatctgacgactgccttgcggatcatgttgaagcatataaagcgcacacagcctcagtgcaggcgttcctcaacatcacagtgggagaacactctattgtgactaccgtgtatgtgaatggagaaactcctgtgaatttcaatggggtcaaaataactgcaggtccgctttccacagcttggacaccctttgatcgcaaaatcgtgcagtatgccggggagatctataattatgattttcctgagtatggggcaggacaaccaggagcatttggagatatacaatccagaacagtctcaagctctgatctgtatgccaataccaacctagtgctgcagagacccaaagcaggagcgatccacgtgccatacactcaggcaccttcgggttttgagcaatggaagaaagataaagctccatcattgaaatttaccgcccctttcggatgcgaaatatatacaaaccccattcgcgccgaaaactgtgctgtagggtcaattccattagcctttgacattcccgacgccttgttcaccagggtgtcagaaacaccgacactttcagcggccgagtgcactcttaacgagtgcgtgtattcttccgactttggtgggatcgccacggtcaagtactcggccagcaagtcaggcaagtgcgcagtccatgtgccatcagggactgctaccctaaaagaagcagcagtcgagctaaccgagcaagggtcggcgactatccatttctcgaccgcaaatatccacccggagttcaggctccaaatatgcacatcatatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgacctgcaggagaatcgatgggcccctataactctctacggctaacctgaatggactacgacatagtctagtccgccaagatgttcccgttccagccaatgtatccgatgcagccaatgccctatcgcaacccgttcgcggccccgcgcaggccctggttccccagaaccgacccttttctggcgatgcaggtgcaggaattaacccgctcgatggctaacctgacgttcaagcaacgccgggacgcgccacctgaggggccatccgctaagaaaccgaagaaggaggcctcgcaaaaacagaaagggggaggccaagggaagaagaagaagaaccaagggaagaagaaggctaagacagggccgcctaatccgaaggcacagaatggaaacaagaagaagaccaacaagaaaccaggcaagagacagcgcatggtcatgaaattggaatctgacaagacgttcccaatcatgttggaagggaagataaacggctacgcttgtgtggtcggagggaagttattcaggccgatgcatgtggaaggcaagatcgacaacgacgttctggccgcgcttaagacgaagaaagcatccaaatacgatcttgagtatgcagatgtgccacagaacatgcgggccgatacattcaaatacacccatgagaaaccccaaggctattacagctggcatcatggagcagtccaatatgaaaatgggcgtttcacggtgccgaaaggagttggggccaagggagacagcggacgacccattctggataaccagggacgggtggtcgctattgtgctgggaggtgtgaatgaaggatctaggacagccctttcagtcgtcatgtggaacgagaagggagttaccgtgaagtatactccggagaactgcgagcaatggtgaatacagcagcaattggcaagctgcttacatagaactcgcggcgattggcatgccgctttaaaatttttattttatttttcttttcttttccgaatcggattttgtttttaatatttc